(19) United States
(12) Patent Application Publication
(10) Pub. No.: US 2025/0073345 A1
(43) Pub. Date: Mar. 6, 2025

(54) ANTIBODY-CONJUGATED CHEMICAL INDUCERS OF DEGRADATION AND METHODS THEREOF

(71) Applicant: Genentech, Inc., South San Francisco, CA (US)

(72) Inventors: Daniel P. SUTHERLIN, Burlingame, CA (US); Donglu ZHANG, San Mateo, CA (US); Summer A. BAKER DOCKERY, San Francisco, CA (US); Peter Scott DRAGOVICH, San Mateo, CA (US)

(73) Assignee: Genentech, Inc., South San Francisco, CA (US)

(21) Appl. No.: 18/784,398

(22) Filed: Jul. 25, 2024

Related U.S. Application Data
(63) Continuation of application No. PCT/US2023/061218, filed on Jan. 25, 2023.
(60) Provisional application No. 63/303,446, filed on Jan. 26, 2022.

(51) Int. Cl. A61K 47/68

(52) U.S. Cl. CPC A61K 47/6803; A61K 47/6849; A61K 47/6889

(57) ABSTRACT

The subject matter described herein is directed to molecules referred to herein as chemical inducers of degradation (CIDEs) and antibody-conjugated CIDEs (Ab-CIDEs), wherein the Ab-CIDEs comprise an antibody covalently bound to the CIDE through a linker, wherein the CIDE can be further covalently bound to a phosphate moiety, and to the uses of the molecules in treating diseases and conditions where targeted protein degradation is beneficial.

### CROSS-REFERENCE TO RELATED APPLICATIONS

This application is a Continuation of International Application No. PCT/US2023/061218, filed Jan. 25, 2023, which claims the benefit of U.S. Provisional Application No. 63/303,446, filed Jan. 26, 2022, the content of each of which is incorporated by reference herein in its entirety.

### FIELD

The subject matter described herein relates generally to degrader conjugates comprising antibody-proteolysis-targeting chimera molecules that are useful for facilitating intracellular degradation of target proteins.

### BACKGROUND

Cell maintenance and normal function requires controlled degradation of cellular proteins. For example, degradation of regulatory proteins triggers events in the cell cycle, such as DNA replication, chromosome segregation, etc. Accordingly, such degradation of proteins has implications for the cell's proliferation, differentiation, and death.

While inhibitors of proteins can block or reduce protein activity in a cell, protein degradation in a cell can also reduce activity or remove altogether the target protein. Utilizing a cell's protein degradation pathway can, therefore, provide a means for reducing or removing protein activity. One of the cell's major degradation pathways is known as the ubiquitin-proteasome system. In this system, a protein is marked for degradation by the proteasome by ubiquitinating the protein. The ubiqitinization of the protein is accomplished by an E3 ubiquitin ligase that binds to a protein and adds ubiquitin molecules to the protein. The E3 ubiquitin ligase is part of a pathway that includes E1 and E2 ubiquitin ligases, which make ubiquitin available to the E3 ubiquitin ligase to add to the protein.

To harness this degradation pathway, molecular constructs known as chemical inducers of degradation (CIDEs) bring together an E3 ubiquitin ligase with a protein that is to be targeted for degradation. To facilitate a protein for degradation by the proteasome, the CIDE is comprised of a group that binds to an E3 ubiquitin ligase and a group that binds to the protein target for degradation. These groups are typically connected with a linker. This CIDE can bring the E3 ubiquitin ligase in proximity with the protein so that it is ubiquitinated and marked for degradation. However, the relatively large size of the CIDE can be problematic for targeted delivery, as well as contribute to undesirable properties, such as fast metabolism/clearance, short half-life, and low bioavailability.

There is an ongoing need in the art for improving CIDEs, including enhancing targeted delivery of CIDEs to cells that contain the protein target. The subject matter described herein addresses this and other shortcomings in the art.

### BRIEF SUMMARY

The present disclosure is directed to a CIDE conjugated or covalently linked to an antibody by a linker. In certain embodiments, the CIDE is phosphorylated. In certain embodiments, the linker covalently attaching the antibody to the CIDE is a peptidomimetic linker. In another aspect, the phosphate moiety acts as a prodrug for the conjugate.

In certain embodiments, the CIDE conjugated or covalently linked to an antibody by a peptidomimetic linker has the chemical structure:

Ab-(L1-D)ⱼ,

where Ab is an antibody; L1 is the linker; D is a CIDE; and j is an integer between 1 and 16. In such aspect, at least one phosphate moiety is covalently bound to D. In certain embodiments, L1 is a peptidomimetic linker.

In certain embodiments, D is a CIDE having the structure: E3LB-L2-PB, where E3LB is an E3 ligase binding ligand covalently bound to L2; L2 is a linker covalently bound to E3LB and PB; and PB is a protein binding group covalently bound to L2. In an aspect of these embodiments, the phosphate moiety is covalently bound to D at the E3LB and/or the PB of D. In an aspect of these embodiments, a single phosphate moiety is covalently bound to D at the E3LB or the PB of D.

In certain embodiments, the E3 ligase is von Hippel-Lindau (VHL) tumor suppressor protein.

In certain embodiments, the phosphate moiety is covalently bound to E3LB of D.

In certain embodiments, the phosphate moiety is covalently bound to PB of D.

In certain embodiments, L1 is covalently bound to PB of D, and the phosphate moiety is covalently bound to E3LB of D.

In certain embodiments, L1 is covalently bound to PB of D, and the phosphate moiety is covalently bound to PB of D.

In certain embodiments, E3LB comprises a hydroxyproline residue, and the phosphate moiety is covalently bound to the hydroxyproline residue.

In certain embodiments, PB comprises a hydroxyphenyl moiety, and the phosphate moiety is covalently bound to the hydroxyphenyl moiety.

In certain embodiments, the PB is a BRM protein.

In certain embodiments, the subject matter described herein is directed to a pharmaceutical composition comprising an Ab-L1-CIDE, as described herein, and one or more pharmaceutically acceptable excipients.

In certain embodiments, the subject matter described herein is directed to the use of an Ab-L1-CIDE, as described herein, in methods of treating conditions and diseases by administering to a subject a pharmaceutical composition comprising an Ab-L1-CIDE.

In certain embodiments, of the subject matter described herein is a method of making an Ab-L1-CIDE.

In certain embodiments, the subject matter described herein is directed to an article of manufacture comprising a pharmaceutical composition comprising an Ab-L1-CIDE, a container, and a package insert or label indicating that the pharmaceutical composition can be used to treat a disease or condition.

Yet other embodiments are also fully described herein.

### DETAILED DESCRIPTION

Disclosed herein, are antibody-conjugated Chemical Inducers of Degradation (“antibody-conjugated CIDE,” “Ab-L1-CIDE” or “Ab-CIDE”) that comprise a linker, such as a peptidomimetic linker, covalently bound to the CIDE and to an antibody, and that are useful in targeted protein degradation. In particular, the present disclosure is directed to antibody-conjugated CIDEs, which contain a ligand that binds to the Von Hippel-Lindau E3 ubiquitin ligase, and a moiety which binds the target protein (such as BRM), such that the target protein is placed in proximity to the ubiquitin ligase to effect degradation, thus, modulating the amount of target protein. In certain embodiments, a phosphate moiety is covalently bound to the CIDE.

The subject matter described herein utilizes antibody targeting to direct a CIDE to a target cell or tissue. As described herein, connecting an antibody to a CIDE to form an Ab-CIDE has been shown to deliver the CIDE to a target cell or tissue. As shown herein, e.g. in the Examples, a cell that expresses an antigen can be targeted by an antigen specific Ab-CIDE, whereby the CIDE portion of the Ab-CIDE is delivered intracellularly to the target cell. Further, CIDEs that comprise an antibody directed to an antigen that is not found on the cell may not result in significant intracellular delivery of the CIDE to the cell.

The disclosed Ab-CIDEs comprise an antibody covalently linked to a Linker 1 (L1), such as a peptidomimetic linker, which is covalently linked at any available point of attachment to a CIDE, in which the CIDE comprises an E3 ubiquitin ligase binding (E3LB) moiety, wherein the E3LB moiety recognizes a E3 ubiquitin ligase protein that is VHL, a Linker 2 (L2) covalently connecting the E3LB moiety to the protein binding moiety (PB), which is the moiety that recognizes a target protein. In certain embodiments, a phosphate moiety is covalently bound to the CIDE. Accordingly, the subject matter described herein is useful for degrading, and thus regulating protein activity, and treating diseases and conditions related to protein activity. In embodiments, phosphorylated Ab-CIDEs result in unexpectedly enhanced in vivo target degradation.

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

### I. Definitions

The term “CIDE” refers to Chemical Inducers of Degradation that are proteolysis-targeting chimera molecules having generally three components, an E3 ubiquitin ligase binding group (E3LB), a linker L2, and a protein binding group (PB). In certain embodiments, the CIDE is phosphorylated.

As used herein, the term “phosphorylated” refers to a CIDE that is covalently bound to a phosphate moiety, which forms a phosphorylated CIDE.

As used herein, the term “phosphate moiety” refers to a chemical group containing a single phosphate and salts thereof. Examples of a phosphate moiety include —(P═O)(OH)₂, —CH₂O(P═O)(OH)₂, —(P═O)(OH)O(P═O)(OH)₂ and —CH₂—O(P═O)(OH)O(P═O)(OH)₂, and salts thereof.

The terms “residue,” “moiety,” “portion,” or “group” refers to a component that is covalently bound or linked to another component. The term “component” is also used herein to describe such a residue, moiety, portion or group. By way of example, a residue of a compound will have an atom or atoms of the compound, such as a hydrogen or hydroxy, replaced with a covalent bond, thereby binding the residue to another component of the CIDE, L1-CIDE or Ab-L1-CIDE. For example a “residue of a CIDE” refers to a CIDE that is covalently linked to one or more groups such as a Linker L2, which itself can be optionally further linked to an antibody.

The term “covalently bound” or “covalently linked” refers to a chemical bond formed by sharing of one or more pairs of electrons.

The term “protein binding group” or “PB” refers to a residue of a small molecule or other compound which is capable of binding to a target protein or other polypeptide target of interest. In the conjugate molecules described herein, the PB binds to the target, which places the target in proximity to a ubiquitin ligase such that degradation of the protein or polypeptide by ubiquitin ligase may occur. As such, the conjugates described herein can include any PB so long as it is covalently bound to L2 and interacts or binds to a target of interest. Non-limiting examples of small molecule target protein binding moieties include compounds that bind BRM (BRAHMA), BRG1, AKT, HPK1, IRE1, Tau and Androgen Receptors (AR), Hsp90 inhibitors, kinase inhibitors, MDM2 inhibitors, compounds targeting Human BET Bromodomain-containing proteins, HDAC inhibitors, human lysine methyltransferase inhibitors, such as KDM5, angiogenesis inhibitors, immunosuppressive compounds, and compounds targeting the aryl hydrocarbon receptor (AHR), among numerous others. In certain embodiments, the CIDES and conjugated CIDEs described herein are not limited to the type of PB. In certain embodiments, the CIDES and conjugated CIDEs described herein are not limited to the type of PB, wherein the PB is covalently bound to L2; and, the CIDE or conjugated CIDE comprises a phosphate moiety and an L1 peptidomimetic linker.

The term “comprises a hydroxyphenyl moiety” and the like refers to PB compound that contains as a portion of its structure, a hydroxyl group covalently bound to a phenyl group, or residue of such a moiety. In certain embodiments, the compounds that contain a hydroxyphenyl (phenol) group or residue thereof bind to BRM.

The term “E3 ligase binding (E3LB) ligand” refers to a molecule that is capable of binding Von Hippel-Lindau (VHL) E3 Ubiquitin Ligase. The terms “VCB E3 Ubiquitin Ligase,” “Von Hippel-Lindau (or VHL) E3 Ubiquitin Ligase,” “VHL,” and “Ubiquitin Ligase,” all generally describe a target enzyme(s) binding site for the E3LB portion of the conjugates described herein. VCB E3 is a protein that in combination with an E2 ubiquitin-conjugating enzyme causes the attachment of ubiquitin to a lysine on a target protein; the E3 ubiquitin ligase targets specific protein substrates for degradation by the proteasome. Thus, E3 ubiquitin ligase alone or in complex with an E2 ubiquitin conjugating enzyme is responsible for the transfer of ubiquitin to targeted proteins. In certain embodiments, the CIDES and conjugated CIDEs described herein are not limited to the type of E3LB. In certain embodiments, the CIDES and conjugated CIDEs described herein are not limited to the type of E3LB, wherein the E3LB is covalently bound to L2; and, the CIDE or conjugated CIDE comprises a phosphate moiety and is further covalently attached to a peptidomimetic L1 linker.

The term “comprises a hydroxyproline residue” and the like refers to a E3LB compound that contains as a portion of its structure, a group or residue of a group having the structure:

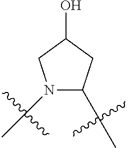 [SMILES: *C1CC(O)CN1*]

Such compounds that are E3 ligase binding (E3LB) ligands are known in the art.

The term “Linker”, “Linker Unit”, or “link” as used herein means a chemical moiety comprising a chain of one or more atoms that covalently attaches a CIDE moiety to an antibody, or a residue, portion, moiety, group or component of a CIDE to another residue, portion, moiety, group or component of the CIDE. In various embodiments, a linker is a divalent radical, specified as Linker 1, Linker 2, L1 or L2.

The term “peptidomimetic” or PM as used herein means a non-peptide chemical moiety. Peptides are short chains of amino acid monomers linked by peptide (amide) bonds, the covalent chemical bonds formed when the carboxyl group of one amino acid reacts with the amino group of another. The shortest peptides are dipeptides, consisting of 2 amino acids joined by a single peptide bond, followed by tripeptides, tetrapeptides, etc. A peptidomimetic chemical moiety includes non-amino acid chemical moieties. A peptidomimetic chemical moiety may also include one or more amino acids that are separated by one or more non-amino acid chemical units. A peptidomimetic chemical moiety may not contain in any portion of its chemical structure, two or more adjacent amino acids that are linked by peptide bonds. A “peptidomimetic linker” is the portion of the molecule that is bound to the CIDE and to the antibody. Useful petpidomimetic linkers are known in the art and others are disclosed herein.

The term “antibody” herein is used in the broadest sense and specifically covers monoclonal antibodies, polyclonal antibodies, dimers, multimers, multispecific antibodies (e.g., bispecific antibodies), and antibody fragments, so long as they exhibit the desired biological activity (Miller et al (2003) Jour. of Immunology 170:4854-4861). Antibodies may be murine, human, humanized, chimeric, or derived from other species. An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by CDRs (complementary determining regions) on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In one aspect, however, the immunoglobulin is of human, murine, or rabbit origin.

The term “antibody fragment(s)” as used herein comprises a portion of a full length antibody, generally the antigen binding or variable region thereof. Examples of antibody fragments include Fab, Fab′, F(ab′)₂, and Fv fragments; diabodies; linear antibodies; minibodies (Olafsen et al (2004) Protein Eng. Design & Sel. 17(4):315-323), fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the subject matter described herein may be made by the hybridoma method first described by Kohler et al (1975) Nature, 256:495, or may be made by recombinant DNA methods (see for example: U.S. Pat. Nos. 4,816,567; 5,807,715). The monoclonal antibodies may also be isolated from phage antibody libraries using the techniques described in Clackson et al (1991) Nature, 352:624-628; Marks et al (1991) J Mol. Biol., 222:581-597; for example.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies of interest herein include “primatized” antibodies comprising variable domain antigen-binding sequences derived from a non-human primate (e.g., Old World Monkey, Ape, etc.) and human constant region sequences.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following. In certain embodiments, an antibody as described herein has dissociation constant (Kd) of ≤1 M, ≤100 nM, ≤10 nM, ≤5 nm, ≤4 nM, ≤3 nM, ≤2 nM, ≤1 nM, ≤0.1 nM, ≤0.01 nM, or ≤0.001 nM (e.g., 10⁻⁸M or less, e.g. from 10⁻⁸ M to 10⁻¹³ M, e.g., from 10⁻⁹ M to 10⁻¹³ M).

The term “free cysteine amino acid” as used herein refers to a cysteine amino acid residue which has been engineered into a parent antibody, has a thiol functional group (—SH), and is not paired as an intramolecular or intermolecular disulfide bridge. The term “amino acid” as used herein means glycine, alanine, valine, leucine, isoleucine, phenylalanine, proline, serine, threonine, tyrosine, cysteine, methionine, lysine, arginine, histidine, tryptophan, aspartic acid, glutamic acid, asparagine, glutamine or citrulline.

A “patient” or “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the patient, individual, or subject is a human. In some embodiments, the patient may be a “cancer patient,” i.e. one who is suffering or at risk for suffering from one or more symptoms of cancer.

A “patient population” refers to a group of cancer patients. Such populations can be used to demonstrate statistically significant efficacy and/or safety of a drug.

The terms “cancer” and “cancerous” refer to or describe the physiological condition in mammals that is typically characterized by unregulated cell growth/proliferation. A “tumor” comprises one or more cancerous cells. Examples of cancer are provided elsewhere herein.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the subject matter described herein are used to delay development of a disease or to slow the progression of a disease.

An “effective amount” of an agent, e.g., a pharmaceutical formulation, refers to an amount effective, at dosages and for periods of time necessary, to achieve the desired therapeutic or prophylactic result. For example, an effective amount of the drug for treating cancer may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. The effective amount may extend progression free survival (e.g. as measured by Response Evaluation Criteria for Solid Tumors, RECIST, or CA-125 changes), result in an objective response (including a partial response, PR, or complete response, CR), increase overall survival time, and/or improve one or more symptoms of cancer (e.g. as assessed by FOSI).

As used herein, the term “therapeutically effective amount” means any amount which, as compared to a corresponding subject who has not received such amount, results in treatment of a disease, disorder, or side effect, or a decrease in the rate of advancement of a disease or disorder. The term also includes within its scope amounts effective to enhance normal physiological function. For use in therapy, therapeutically effective amounts of an Ab-CIDE, as well as salts thereof, may be administered as the raw chemical. Additionally, the active ingredient may be presented as a pharmaceutical composition.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable excipient” refers to an ingredient in a pharmaceutical formulation, other than an active ingredient, which is nontoxic to a subject. A pharmaceutically acceptable excipient includes, but is not limited to, a buffer, carrier, stabilizer, or preservative.

The phrase “pharmaceutically acceptable salt,” as used herein, refers to pharmaceutically acceptable organic or inorganic salts of a molecule. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

Other salts, which are not pharmaceutically acceptable, may be useful in the preparation of compounds of described herein and these should be considered to form a further aspect of the subject matter. These salts, such as oxalic or trifluoroacetate, while not in themselves pharmaceutically acceptable, may be useful in the preparation of salts useful as intermediates in obtaining the compounds described herein and their pharmaceutically acceptable salts.

The term “alkyl” as used herein refers to a saturated linear or branched-chain monovalent hydrocarbon radical of any length from one to six carbon atoms (C₁-C₆), wherein the alkyl radical may be optionally substituted independently with one or more substituents described below. In another embodiment, an alkyl radical is one, two, three, four or five carbon atoms. Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), and the like.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆-C₂₀ aryl), 6 to 12 carbon ring atoms (i.e., C₆-C₁₂ aryl), or 6 to 10 carbon ring atoms (i.e., C₆-C₁₀ aryl). Examples of aryl groups include, e.g., phenyl, naphthyl, fluorenyl and anthryl.

“Heteroaryl” refers to an aromatic group having a single ring, multiple rings or multiple fused rings, with one or more ring heteroatoms independently selected from nitrogen, oxygen, and sulfur. As used herein, heteroaryl includes 1 to 20 ring carbon atoms (i.e., C₁-C₂₀ heteroaryl), 3 to 12 ring carbon atoms (i.e., C₃-C₁₂ heteroaryl), or 3 to 8 carbon ring atoms (i.e., C₃-C₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. In certain instances, heteroaryl includes 9-10 membered ring systems, 6-10 membered ring systems, 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

The term “alkylene” as used herein refers to a saturated linear or branched-chain divalent hydrocarbon radical of any length from one to twelve carbon atoms (C₁-C₁₂), wherein the alkylene radical may be optionally substituted independently with one or more substituents described below. In another embodiment, an alkylene radical is one to eight carbon atoms (C₁-C₈), or one to six carbon atoms (C₁-C₆). Examples of alkylene groups include, but are not limited to, methylene (—CH₂—), ethylene (—CH₂CH₂—), propylene (—CH₂CH₂CH₂—), and the like.

The terms “carbocycle”, “carbocyclyl”, “carbocyclic ring” and “cycloalkyl” refer to a monovalent non-aromatic, saturated or partially unsaturated ring having 3 to 5 carbon atoms (C₃-C₅) as a monocyclic ring. Examples of monocyclic carbocycles include, but are not limited to, cyclopropyl, cyclobutyl, cyclopentyl, 1-cyclopent-1-enyl, 1-cyclopent-2-enyl, 1-cyclopent-3-enyl, and the like. Carbocyclyl groups can be optionally substituted independently with one or more alkyl groups.

“Heterocycle,” “heterocyclic,” “heterocycloalkyl” or “heterocyclyl” refers to a saturated or partially unsaturated group having a single ring or multiple condensed rings, including fused, bridged, or spiro ring systems, and having from 3 to 20 ring atoms, including 1 to 10 hetero atoms. These ring atoms are selected from the group consisting of carbon, nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for N-oxide, —S(O)—, or —SO₂— moieties. Examples of heterocycles include, but are not limited to, azetidine, dihydroindole, indazole, quinolizine, imidazolidine, imidazoline, piperidine, piperazine, indoline, 1,2,3,4-tetrahydroisoquinoline, thiazolidine, morpholinyl, thiomorpholinyl (also referred to as thiamorpholinyl), 1,1-dioxothiomorpholinyl, piperidinyl, pyrrolidine, tetrahydrofuranyl, and the like. A heterocyclyl group can be substituted as described in WO2014/100762.

The term “chiral” refers to molecules which have the property of non-superimposability of the mirror image partner, while the term “achiral” refers to molecules which are superimposable on their mirror image partner. The term “stereoisomers” refers to compounds which have identical chemical constitution, but differ with regard to the arrangement of the atoms or groups in space. “Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography. “Enantiomers” refer to two stereoisomers of a compound which are non-superimposable mirror images of one another. Stereochemical definitions and conventions used herein generally follow S. P. Parker, Ed., McGraw-HillDictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

Additional definitions and abbreviations may be provided elsewhere herein.

### II. Chemical Inducers of Degradation

The present disclosure is directed to a CIDE conjugated or covalently linked to an antibody by a linker. The present disclosure is directed to a CIDE conjugated or covalently linked to an antibody by a peptidomimetic linker. In certain embodiments, the CIDE is phosphorylated. In another aspect, the phosphate moiety acts as a prodrug for the conjugate.

In embodiments, the CIDE has the structure: E3LB-L2-PB, where E3LB is an E3 ligase binding ligand covalently bound to L2; L2 is a linker covalently bound to E3LB and PB; and PB is a protein binding group covalently bound to L2. In aspects, a phosphate moiety is covalently bound to E3LB and/or PB of CIDE, D.

In certain embodiments, the E3 ligase is von Hippel-Lindau (VHL) tumor suppressor protein.

In certain embodiments, the phosphate moiety is covalently bound to E3LB of D.

In certain embodiments, the phosphate moiety is covalently bound to PB of D.

In certain embodiments, the L1 is covalently bound to PB of D, and the phosphate moiety is covalently bound to E3LB of D.

In certain embodiments, the L1 is covalently bound to PB of D, and the phosphate moiety is covalently bound to PB of D.

In certain embodiments, the E3LB comprises a hydroxyproline residue, and the phosphate moiety is covalently bound to the hydroxyproline residue.

In certain embodiments, PB comprises a hydroxyphenyl moiety, and the phosphate moiety is covalently bound to the hydroxyphenyl moiety.

In certain embodiments, the PB is a BRM protein.

In certain embodiments, the CIDE conjugated or covalently linked to an antibody by a peptidomimetic linker L1 has the formula:

Ab-(L1-D)ⱼ,

where Ab is an antibody; L1 is the linker; D is a CIDE; and j is an integer between 1 and 16. In such aspect, at least one phosphate moiety is covalently bound to D.

The following sections describe in further detail the above components that comprise the Ab-CIDE. To obtain an Ab-CIDE having one or more properties that include potent efficacy, a desirable therapeutic index, the following components are provided:

### 1. Antibody (Ab)

As described herein, antibodies, e.g., a monoclonal antibodies (mABs) are used to deliver a CIDE to target cells, e.g., cells that express the specific protein that is targeted by the antibody. The antibody portion of an Ab-CIDE can target a cell that expresses an antigen whereby the antigen specific Ab-CIDE is delivered intracellularly to the target cell, typically through endocytosis. In some instances, pinocytocis or similar non-specific routes of uptake may result in general cellular uptake of the Ab-CIDE within antigen expressing or non-expressing cells. The Ab-CIDEs and method of their use described herein advantageously utilize antibody recognition of the cellular surface and/or endocytosis of the Ab-CIDE to deliver the CIDE portion inside cells. Antibodies are described in WO2020/086858, which is herein incorporated by reference in its entirety.

In particular embodiments, the antibody may be mutated to reduce effector function. Examples of mutations that modulate the Fc effector function include LALAPG mutations and NG2LH mutations.

In particular embodiments, the antibody is a THIOMAB™ as previously described in WO2016/04856. Further, combinations are contemplated, such that any antibody target can be combined with any suitable combination of THIOMAB™ mutations with or without any Fc effector modulation including LALAPG or NG2LH mutations.

The antibody can be a human antibody, for example, as described generally in van Dijk and van de Winkel, Curr. Opin. Pharmacol. 5: 368-74 (2001) and Lonberg, Curr. Opin. Immunol. 20:450-459 (2008). The antibody can be a library-derived antibody. A variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, NJ, 2001) and further described, e.g., in the McCafferty et al., Nature 348:552-554; Clackson et al., Nature 352: 624-628 (1991); Marks et al., J Mol. Biol. 222: 581-597 (1992); Marks and Bradbury, in Methods in Molecular Biology 248:161-175 (Lo, ed., Human Press, Totowa, NJ, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

The antibody can be a chimeric and humanized antibody. Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

The antibody can be a multispecific antibody, e.g. a bispecific antibody. The term “multispecific antibody” as used herein refers to an antibody comprising an antigen-binding domain that has polyepitopic specificity (i.e., is capable of binding to two, or more, different epitopes on one molecule or is capable of binding to epitopes on two, or more, different molecules). The term “bispecific antibody” as used herein refers to a multispecific antibody comprising an antigen-binding domain that is capable of binding to two different epitopes on one molecule or is capable of binding to epitopes on two different molecules. A bispecific antibody may also be referred to herein as having “dual specificity” or as being “dual specific.” Exemplary bispecific antibodies may bind both protein and any other antigen. Techniques for making multispecific antibodies include, but are not limited to, recombinant co-expression of two immunoglobulin heavy chain-light chain pairs having different specificities (see Milstein and Cuello, Nature 305: 537 (1983), WO 93/08829, and Traunecker et al., EMBO J. 10: 3655 (1991)), and “knob-in-hole” engineering (see, e.g., U.S. Pat. No. 5,731,168, WO2009/089004, US2009/0182127, US2011/0287009, Marvin and Zhu, Acta Pharmacol. Sin. (2005) 26(6):649-658, and Kontermann (2005) Acta Pharmacol. Sin., 26:1-9). Multispecific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J Immunol., 152:5368 (1994)); and preparing trispecific antibodies as described, e.g., in Tutt et al. J Immunol. 147: 60 (1991). Engineered antibodies with three or more functional antigen binding sites, including “Octopus antibodies” or “dual-variable domain immunoglobulins” (DVDs) are also included herein (see, e.g., US 2006/0025576A1, and Wu et al. Nature Biotechnology (2007)).). The antibody or fragment herein also includes a “Dual Acting FAb” or “DAF” comprising an antigen binding site that binds to a target protein as well as another, different antigen (see, US 2008/0069820, for example).

The antibody can be an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat. Med 9:129-134 (2003); and Hollinger et al., Proc. Natl. Acad Sci. USA 90: 6444-6448 (1993). Triabodies and tetrabodies are also described in Hudson et al., Nat. Med 9:129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1). Antibody fragments can be made by various techniques, including but not limited to proteolytic digestion of an intact antibody as well as production by recombinant host cells.

The antibody can be an antibody variant. In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

The antibody can be produced using recombinant methods and compositions, e.g., as described in U.S. Pat. No. 4,816,567. Referring now to antibody affinity, in embodiments, the antibody binds to one or more tumor-associated antigens or cell-surface receptors.

In certain embodiments, the tumor-associated antigen or cell surface receptor is selected from CD71, Trop2, MSLN, NaPi2b, Ly6E, EpCAM, STEAP1, HER2, CD33 and CD22. As such, in certain embodiments, an Ab-CIDE may comprise an antibody selected from: antianti-Ly6E antiantibodies, anti-NaPi2b antibodies, anti-CD22 antibodies, anti-CD71 antibodies, anti-Trop2 antibodies, anti-MSLN antibodies, anti-EpCAM antibodies, anti-Steap1 antibodies, anti-CD33 antibodies, anti-CLL1 antibodies, anti-CD123 antibodies and anti-HER2 antibodies.

Particular antibodies include but are not limited to:

i. Anti-Ly6E Antibodies

In certain embodiments, an Ab-CIDE can comprise anti-Ly6E antibodies.: Ly6E (lymphocyte antigen 6 complex, locis E; Ly67,RIG-E,SCA-2,TSA-1); NP_002337.1; NM_002346.2; de Nooij-van Dalen, A. G. et al (2003) Int. J. Cancer 103 (6), 768-774; Zammit, D. J. et al (2002) Mol. Cell. Biol. 22 (3):946-952; WO 2013/17705.

Ly6E is a GPI linked, 131 amino acid length, ˜8.4 kDa protein of unknown function with no known binding partners. It was initially identified as a transcript expressed in immature thymocyte, thymic medullary epithelial cells in mice (Mao, et al. (1996) Proc. Natl. Acad. Sci. U.S.A. 93:5910-5914). In some embodiments, the subject matter described herein provides an Ab-CIDE comprising an anti-Ly6E antibody described in PCT Publication No. WO 2013/177055.

ii. Anti-NaPi2b Antibodies

In certain embodiments, an Ab-CIDE comprises anti-NaPi2b antibodies: Napi2b (Napi3b, NAPI-3B, NPTIIb, SLC34A2, solute carrier family 34 (sodium phosphate), member 2, type II sodium-dependent phosphate transporter 3b, Genbank accession no. NM_006424) J. Biol. Chem. 277 (22):19665-19672 (2002), Genomics 62 (2):281-284 (1999), Feild, J. A., et al (1999) Biochem. Biophys. Res. Commun. 258 (3):578-582); WO2004022778 (Claim 2); EP1394274 (Example 11); WO2002102235 (Claim 13; Page 326); EP875569 (Claim 1; Page 17-19); WO200157188 (Claim 20; Page 329); WO2004032842 (Example IV); WO200175177 (Claim 24; Page 139-140); Cross-references: MIM:604217; NP_006415.1; NM_006424_1.

iii. Anti-CD22 Antibodies

In certain embodiments, an Ab-CIDE can comprise anti-CD22 antibodies: CD22 (B-cell receptor CD22-B isoform, BL-CAM, Lyb-8, Lyb8, SIGLEC-2, FLJ22814, Genbank accession No. AK026467); Wilson et al (1991) J. Exp. Med. 173:137-146; WO2003072036 (Claim 1; FIG. 1); Cross-references: MIM:107266; NP_001762.1; NM_001771_1

iv. Anti-CD71 Antibodies

In certain embodiments, an Ab-CIDE can comprise anti-CD71 antibodies. CD71 (transferrin receptor) is an integral membrane glycoprotein that plays an important role in cellular uptake of iron. It is well known as a marker for cell proliferation and activation. Although all proliferating cells in hematopoietic system express CD71, however, CD71 has been considered as a useful erythroid-associated antigen. In one embodiment, an anti-CD71 antibody is described in: WO2016081643 which is incorporated by reference in its entirety.

v. Anti-Trop2 Antibodies

In certain embodiments, an Ab-CIDE can comprise anti-Trop2 antibodies. Trop2 (trophoblast antigen 2) is a transmembrane glycoprotein that is an intracellular calcium signal transducer that is differentially expressed in many cancers. It signals cells for self-renewal, proliferation, invasion, and survival. Trop 2 is also known as cell surface glycoprotein Trop-2/Trop2, gastrointestinal tumor-associated antigen GA7331, pancreatic carcinoma marker protein GA733-1/GA733, membrane component chromosome 1 surface marker 1 M1S1, epithelial glycoprotein-1, EGP-1, CAA1, Gelatinous Drop-Like Corneal Dystrophy GDLD, and TTD2. In any of the above embodiments, an anti-Trop2 antibody of an Ab-CIDE is humanized. In one embodiment, the anti-Trop2 antibodies are described in US-2014/0377287 and US-2015/0366988, each of which is incorporated by reference in its entirety.

vi. Anti-MSLN Antibodies

In certain embodiments, an Ab-CIDE can comprise anti-MSLN antibodies. MSLN (mesothelin) is a glycosylphosphatidylinositol-anchored cell-surface protein that may function as a cell adhesion protein. MSLN is also known as CAK1 and MPF. This protein is overexpressed in epithelial mesotheliomas, ovarian cancers and in specific squamous cell carcinomas. In any of the above embodiments, an anti-MSLN antibody of an Ab-CIDE is humanized. In one embodiment, the anti-MSLN antibody is h7D9.v3 described in Scales, S. J. et al., Mol. Cancer Ther. 2014, 13(11), 2630-2640, which is incorporated by reference in its entirety.

vii. Anti-EpCAM Antibodies

In certain embodiments, an Ab-CIDE can comprise anti-EpCAM antibodies. In an aspect, the antibody of the Ab-CIDE may be an antibody that is directed to a protein that is found on numerous cells or tissue types. Examples of such antibodies include EpCAM. Epithelial cell adhesion molecule (EpCAM) is a transmembrane glycoprotein mediating Ca2+-independent homotypic cell-cell adhesion in epithelia (Litvinov, S. et al. (1994) Journal of Cell Biology 125(2):437-46). Also known as DIAR5, EGP-2, EGP314, EGP40, ESA, HNPCC8, KS1/4, KSA, M4S1, MIC18, MK-1, TACSTD1, TROP1, EpCAM is also involved in cell signaling, (Maetzel, D. et al. (2009) Nature Cell Biology 11(2):162-71), migration (Osta, W A; et al. (2004) Cancer Res. 64(16):5818-24), proliferation, and differentiation (Litvinov, S. et al. (1996) Am J Pathol. 148(3):865-75). Additionally, EpCAM has oncogenic potential via its capacity to upregulate c-myc, e-fabp, and cyclins A & E (Munz, M. et al. (2004) Oncogene 23(34):5748-58). Since EpCAM is expressed exclusively in epithelia and epithelial-derived neoplasms, EpCAM can be used as a diagnostic marker for various cancers. In other words, an Ab-CIDE can be used to deliver a CIDE to many cells or tissues rather than specific cell types or tissue types as when using a using a targeted antibody.

viii. Anti-Steap1 Antibodies

In certain embodiments, Ab-CIDEs comprise anti-STEAP1 antibodies. STEAP1 (six transmembrane epithelial antigen of prostate, Genbank accession no. NM_0 12449) Cancer Res. 6 1 (15), 5857-5860 (2001), Hubert, R. S., et al (1999) Proc. Natl. Acad. Sci. U.S.A. 96 (25): 14523-14528); WO2004065577 (Claim 6); WO2004027049 (FIG. 1L); EP1394274 (Example 11); WO2004016225 (Claim 2); WO2003042661 (Claim 12); US2003 157089 (Example 5); US2003 185830 (Example 5); US2003064397 (FIG. 2); WO200289747 (Example 5; Page 618-619); WO2003022995 (Example 9; FIG. 13A, Example 53; Page 173, Example 2; FIG. 2A);

viii. Anti-Steap2 Antibodies

In certain embodiments, Ab-CIDEs comprise anti-STEAP2 antibodies. STEAP2 (HGNC 8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein, Genbank accession no. AF455138) Lab. Invest. 82 (11): 1573 1582 (2002)); WO2003087306; US2003064397 (Claim 1; FIG. 1); WO200272596 (Claim 13; Page 54-55); WO200172962 (Claim 1; FIG. 4B); WO2003 104270 (Claim 11); WO2003 104270 (Claim 16); US2004005598 (Claim 22); WO2003042661 (Claim 12); US2003060612 (Claim 12; FIG. 10); WO200226822 (Claim 23; FIG. 2); WO200216429 (Claim 12; FIG. 10); Cross-references: GF22655488; AAN04080.1; AF455138_I

ix. Anti-Her2 Antibodies

In Ab-CIDEs comprise anti-HER2 antibodies. In one embodiment an anti-HER2 antibody of the Ab-CIDE comprises a humanized anti-HER2 antibody. In some embodiments, the Ab-CIDE comprises a humanized HER2 antibody also referred to as trastuzumab, commercially available under the tradename iERCEPTIN®. In another embodiment, an anti-HER2 antibody of a Ab-CIDE comprises a humanized anti-iER2 antibody, e.g., humanized 2C4, as described in U.S. Pat. No. 7,862,817. An exemplary humanized 2C4 antibody is pertuzumab, commercially available under the tradename PERJETA®.

x. Anti CD33 Antibodies

In certain embodiments, Ab-CIDEs comprise anti-CD33 antibodies. CD33, a member of the sialic acid binding, immunoglobulin-like lectin family, is a 67 kDa glycosylated transmembrane protein. CD33 is expressed on most myeloid and monocytic leukemia cells in addition to committed myelomonocytic and erythroid progenitor cells. It is not seen on the earliest pluripotent stem cells, mature granulocytes, lymphoid cells, or nonhematopoietic cells (Sabbath et al., (1985). Clin. Invest. 75:756-56; Andrews et al., (1986) Blood 68:1030-5). CD33 contains two tyrosine residues on its cytoplasmic tail, each of which is followed by hydrophobic residues similar to the immunoreceptor tyrosine-based inhibitory motif (ITTIM) seen in many inhibitory receptors.

### 2. Linkers (L1)

As described herein, a linker (L1), which is a bifunctional or multifunctional moiety that can be used to link one or more CIDE moieties (D) to an antibody (Ab) to form an Ab-CIDE. In some embodiments, Ab-CIDEs can be prepared using a linker (L1) having reactive functionalities for covalently attaching to the CIDE and to the antibody. For example, in some embodiments, a cysteine thiol of an antibody (Ab) can form a bond with a reactive functional group of a linker or a linker L1-CIDE group to make an Ab-CIDE.

i. Peptidomimetic Linkers

In certain embodiments, the linker (L1) of the Ab-CIDE is a non-peptide, peptidomimetic linker that is cleavable by lysosomal enzymes. In such embodiments, the non-peptide linkers act like peptides and may be effectively cleaved by lysosomal proteases. For example, the amide bond in the middle of a dipeptide (e.g. Val-Cit) may be replaced with an amide mimic, and/or entire amino acid (e.g., valine amino acid in Val-Cit dipeptide) may be replaced with a non-amino acid moiety (e.g., cycloalkyl dicarbonyl structures (for example, a cycloalkyl having a ring size of 4 or 5)).

In certain embodiments, L1 is a peptidomimetic linker having the following formula:

-Str-(PM)-Sp,
wherein:
Str is a stretcher unit covalently attached to Ab;
Sp is a bond or spacer unit covalently attached to a CIDE moiety; and
PM is a non-peptide chemical moiety selected from the group consisting of:

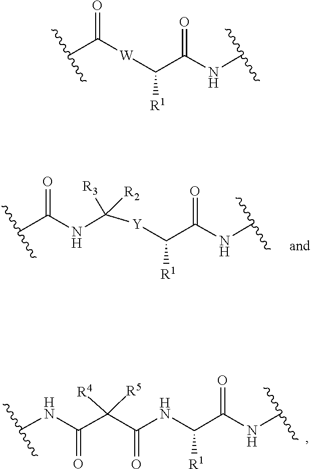

wherein
W is —NH-heterocycloalkyl- or heterocycloalkyl;
Y is heteroaryl, aryl, —C(O)C₁-C₆alkylene, C₁-C₆alkylene-NH₂, C₁-C₆alkylene-NH—CH₃, C₁-C₆alkylene-N—(CH₃)₂, C₁-C₆alkenyl or C₁-C₆alkylenyl;
each R¹ is independently C₁-C₁₀alkyl, C₁-C₁₀alkenyl, (C₁-C₁₀alkyl)NHC(NH)NH₂ or (C₁-C₁₀alkyl)NHC(O)NH₂ —CH₂CH₂CH₂CH₂NH₂; —CH₂CH₂CH₂CH₂NH—CH₃; and —CH₂CH₂CH₂CH₂N—(CH₃)₂;
R³ and R² are each independently H, C₁-C₁₀alkyl, C₁-C₁₀alkenyl, arylalkyl or heteroarylalkyl, or R³ and R² together may form a C₃-C₇cycloalkyl; and
R⁴ and R⁵ are each independently C₁-C₁₀alkyl, C₁-C₁₀alkenyl, arylalkyl, heteroarylalkyl, (C₁-C₁₀alkyl)OCH₂—, or R⁴ and R⁵ may form a C₃-C₇cycloalkyl ring.

L1 may be connected to the CIDE through any of the E3LB, L2 or PB groups.

In certain embodiments, Y is heteroaryl; and R⁴ and R⁵ together form a cyclobutyl ring.

In certain embodiments, Y is a moiety selected from the group consisting of:

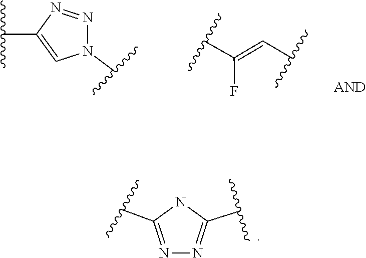

In certain embodiments, Str is a chemical moiety represented by the following formula:

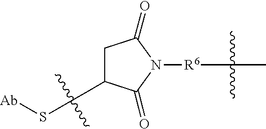

wherein R⁶ is selected from the group consisting of C₁-C₁₀alkylene, C₁-C₁₀alkenyl, C₃-C₈cycloalkyl, (C₁-C₈alkylene)O—, and C₁-C₁₀alkylene-C(O)N(R^{a})—C₂-C₆alkylene, where each alkylene may be substituted by one to five substituents selected from the group consisting of halo, trifluoromethyl, difluoromethyl, amino, alkylamino, cyano, sulfonyl, sulfonamide, sulfoxide, hydroxy, alkoxy, ester, carboxylic acid, alkylthio, C₃-C₈cycloalkyl, C₄-C₇heterocycloalkyl, aryl, arylalkyl, heteroarylalkyl and heteroaryl each R^{a} is independently H or C₁-C₆alkyl; Sp is —Ar—R^{b}—, wherein Ar is aryl or heteroaryl, R^{b} is (C₁-C₁₀alkylene)O—. Conjugation to the antibody can occur as the maleimide reacts via Michael addition with an exposed Cys residue on the antibody. The exposed Cys residue can either be artificially introduced by molecular engineering and/or produced by reduction of the interchain disulfide bonds)

In certain embodiments, Str has the formula:

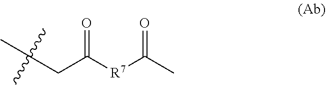

wherein R⁷ is selected from C₁-C₁₀alkylene, C₁-C₁₀alkenyl, (C₁-C₁₀alkylene)O—, N(R^{c})—(C₂-C₆ alkylene)-N(R^{c}) and N(R^{c})—(C₂-C₆alkylene); where each R^{c} is independently H or C₁-C₆ alkyl; Sp is —Ar—R^{b}—, wherein Ar is aryl or heteroaryl, R^{b} is (C₁-C₁₀alkylene)O— or Sp-C₁-C₆alkylene-C(O)NH—.

In embodiments, L1 is a non-peptide chemical moiety represented by the following formula

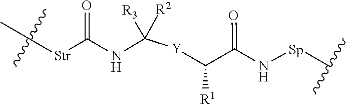

R¹ is C₁-C₆alkyl, C₁-C₆alkenyl, (C₁-C₆alkyl)NHC(NH)NH₂ or (C₁-C₆alkyl)NHC(O)NH₂;
R³ and R² are each independently H or C₁-C₁₀alkyl.

In embodiments, L1 is a non-peptide chemical moiety represented by the following formula

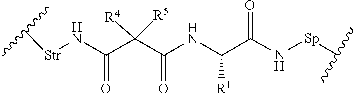

R¹ is C₁-C₆ alkyl, (C₁-C₆alkyl)NHC(NH)NH₂ or (C₁-C₆alkyl)NHC(O)NH₂;
R⁴ and R⁵ together form a C₃-C₇cycloalkyl ring.

In embodiments, L1 is a non-peptide chemical moiety represented by the following formula

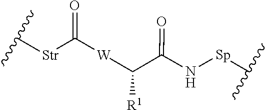

R¹ is C₁-C₆alkyl, (C₁-C₆alkyl)NHC(NH)NH₂ or (C₁-C₆alkyl)NHC(O)NH₂ and W is as defined above.

In some embodiments, the linker may be a peptidomimetic linker such as those described in WO2015/095227, WO2015/095124 or WO2015/095223, each of which is hereby incorporated by reference in its entirety.

In certain embodiments, L1 is selected from the group consisting of:
i)

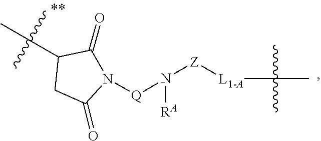

wherein

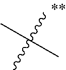

indicates the point of attachment to Ab;
Z is —(CH₂)ₚ— or —CH₂—(CH₂—O—CH₂)ₚ—CH₂—, wherein p is 1, 2, 3, 4, 5 or 6;
R^{A} is hydrogen, C₁₋₆ alkyl, or —(CH₂)ᵥ-aryl, wherein v is 0 or 1; such as phenyl or benzyl;
Q is selected from the group consisting of:
a)

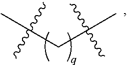

wherein, q is 1, 2, 3 or 4; and
b)

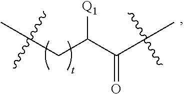

wherein t is 0, 1, 2, 3 or 4;
Q¹ is hydrogen,

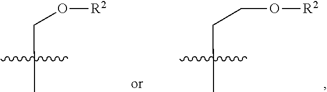

wherein R² is hydrogen, halo(C₁₋₆)alkyl or C₁₋₆ alkyl; and
L_{1-A} is:

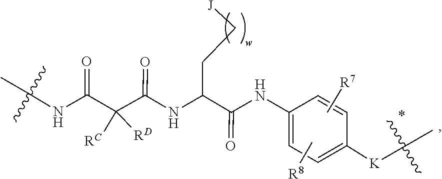

wherein,

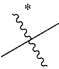

indicates the attachment point to D; and,

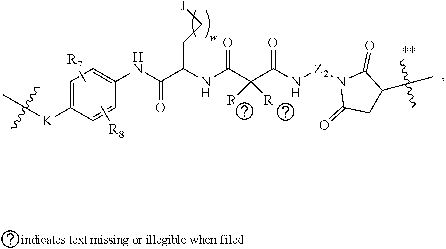

wherein

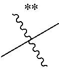

indicates the attachment point to Ab;
Z₂ is a C₁₋₁₂ alkylene or —[CH₂]_{g}—O—[CH₂]ₕ—, wherein g and h are each independently 0, 1 or 2;
w is 0, 1, 2, 3, 4 or 5;
J is selected from the group consisting of C₁₋₅ alkyl, —N(Rₓ)(R_{y}), —C(O)NH₂, —NH—C(O)—NH₂, and —NHC(═NH)NH₂, wherein, Rₓ and R_{y} are each independently coselected from hydrogen and C₁₋₃alkyl;
K is selected from the group consisting of C₁₋₃alkylene, —CH(R)—, —C(O)—, —C(O)—O—CH(R)—, —CH₂—O—C(O)—, —CH₂—O—C(O)—NH—CH₂—, and —CH₂—O—C(O)—R—[CH₂]_{q}—O—, wherein R is hydrogen, C₁₋₃alkyl, N(Rₓ)(R_{y}), —O—N(Rₓ)(R_{y}) or C(O)—N(Rₓ)(R_{y}), wherein q is 0, 1, 2, or 3, and wherein Rₓ and R_{y} are each independently selected from hydrogen and C₁₋₃alkyl, or Rₓ and R_{y} together with the nitrogen to which each is attached form an optionally substituted 5- to 7-member heterocyclyl;
Rₐ, R_{b}, R^{C} and R^{D} are each independently selected from hydrogen and C₁₋₃alkyl, or Rₐ and R_{b} or R^{C} and R^{D}, together with the carbon to which each is attached, form an optionally substituted C₃₋₆cycloalkyl; and
R₇ and R₈ are each independently hydrogen, halo, C₁₋₅ alkyl, C₁₋₅ alkoxy or hydroxyl.

In certain embodiments of L1-A, when w is 0 and J is a methyl L₁a comprises:

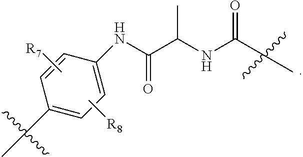

In certain embodiment, K of L1 is selected from the group consisting of:

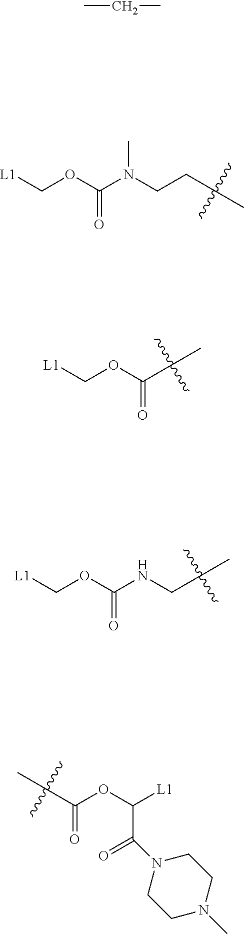

In certain embodiments, L1 is selected from the group consisting of:

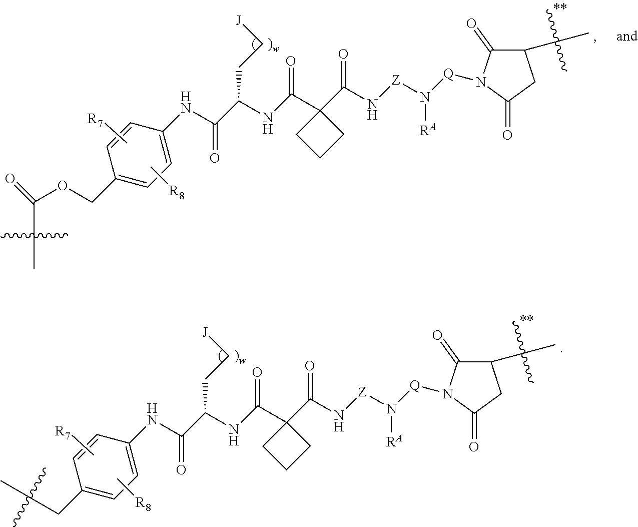

In certain embodiments, R₇ and R₈ are each hydrogen.

In certain embodiments, Z is —(CH₂)ₚ—.

In certain embodiments, w is 2 or 3, and J is —N(CH₃)₂ or —NH(CO)NH.

In certain embodiments, Q is —CH₂—CH₂—.

In certain embodiments, L1 is selected from the group consisting of:

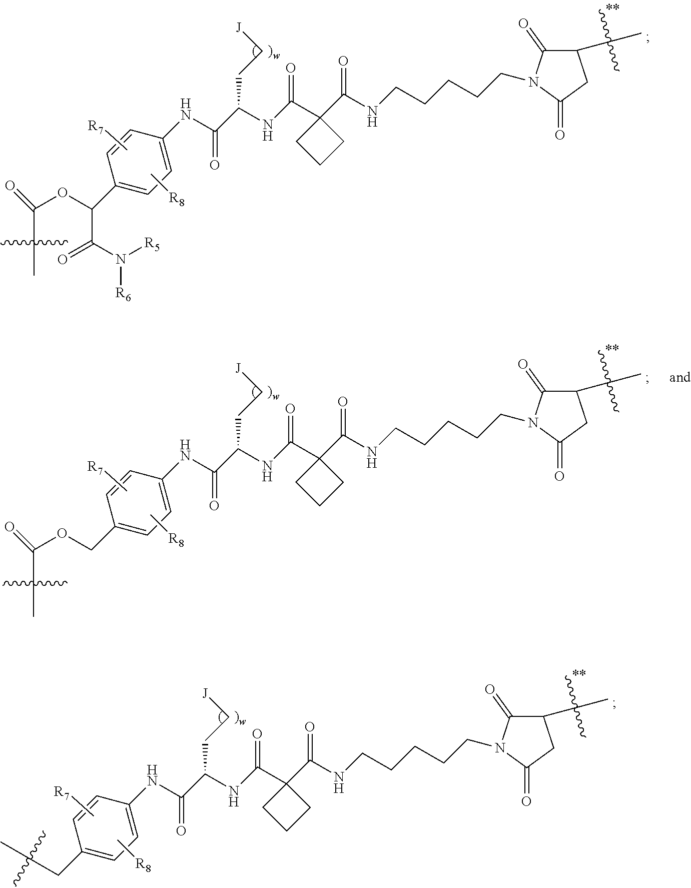

wherein,
R₅ and R₆ are independently hydrogen or C₁₋₅ alkyl; or R₅ and R₆ together with the nitrogen to which each is attached form an optionally substituted 5- to 7-member heterocyclyl.

In certain embodiments, L1 is a peptidomimetic linker selected from the group consisting of:

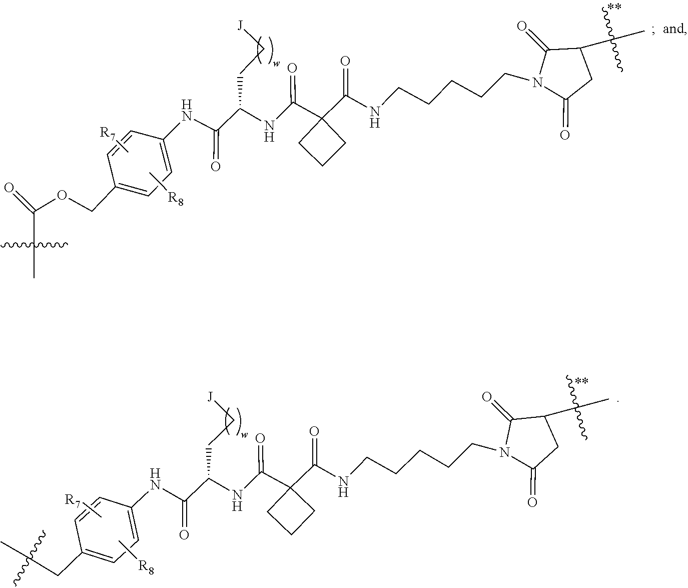

In certain embodiments, J is —NH—C(O)—NH₂ or —N(CH₃)₂.

In certain embodiments, w is 2 and J is —NH—C(O)—NH₂; or w is 3 and J is —N(CH₃)₂.

ii. Other Linkers

In certain embodiments, L1 can be a peptide linker or other linker. Peptide linkers, such as Valine-Citrulline (Val-Cit), that can be hydrolyzed by lysosomal enzymes (such as Cathepsin B) have been used to connect the drug with the antibody (U.S. Pat. No. 6,214,345). They have been useful, due in part to their relative stability in systemic circulation and the ability to efficiently release the drug in tumor.

In one aspect the carbonyl group of the linker is connected to an amine group in the CIDE. It is also noted that the sulfur atom connected to Ab is a sulfur group from a cysteine in the antibody. In another aspect, a linker L1 has a functionality that is capable of reacting with a free cysteine present on an antibody to form a covalent bond. Nonlimiting examples of such reactive functionalities include maleimide, haloacetamides, a-haloacetyl, activated esters such as succinimide esters, 4-nitrophenyl esters, pentafluorophenyl esters, tetrafluorophenyl esters, anhydrides, acid chlorides, sulfonyl chlorides, isocyanates, and isothiocyanates. See, e.g., the conjugation method at page 766 of Klussman, et al (2004), Bioconjugate Chemistry 15(4):765-773, and the Examples herein.

In some embodiments, a linker has a functionality that is capable of reacting with an electrophilic group present on an antibody. Examples of such electrophilic groups include, but are not limited to, aldehyde and ketone carbonyl groups. In some embodiments, a heteroatom of the reactive functionality of the linker can react with an electrophilic group on an antibody and form a covalent bond to an antibody unit. Nonlimiting examples of such reactive functionalities include, but are not limited to, hydrazide, oxime, amino, hydrazine, thiosemicarbazone, hydrazine carboxylate, and arylhydrazide.

A linker may comprise one or more linker components. Exemplary linker components include 6-maleimidocaproyl (“MC”), maleimidopropanoyl (“MP”), valine-citrulline (“val-cit” or “vc”), alanine-phenylalanine (“ala-phe”), p-aminobenzyloxycarbonyl (a “PAB”), N-Succinimidyl 4-(2-pyridylthio) pentanoate (“SPP”), and 4-(N-maleimidom ethyl) cyclohexane-1 carboxylate (“MCC”). Various linker components are known in the art, some of which are described below.

A linker may be a“cleavable linker,” facilitating release of a CIDE. Nonlimiting exemplary cleavable linkers include acid-labile linkers (e.g, comprising hydrazone), protease-sensitive (e.g, peptidase-sensitive) linkers, photolabile linkers, or disulfide-containing linkers (Chari et al., Cancer Research 52: 127-131 (1992); U.S. Pat. No. 5,208,020).

In certain embodiments, a linker has the following Formula:

-Aₐ-W_{w}—Y_{y}—
wherein A is a“stretcher unit”, and a is an integer from 0 to 1; W is an “amino acid unit”, and w is an integer from 0 to 12; Y is a “spacer unit”, and y is 0, 1, or 2. Exemplary embodiments of such linkers are described in U.S. Pat. No. 7,498,298.

In some embodiments, a linker component comprises a“stretcher unit” that links an antibody to another linker component or to a CIDE moiety. Nonlimiting exemplary stretcher units are shown below (wherein the wavy line indicates sites of covalent attachment to an antibody, CIDE, or additional linker components):

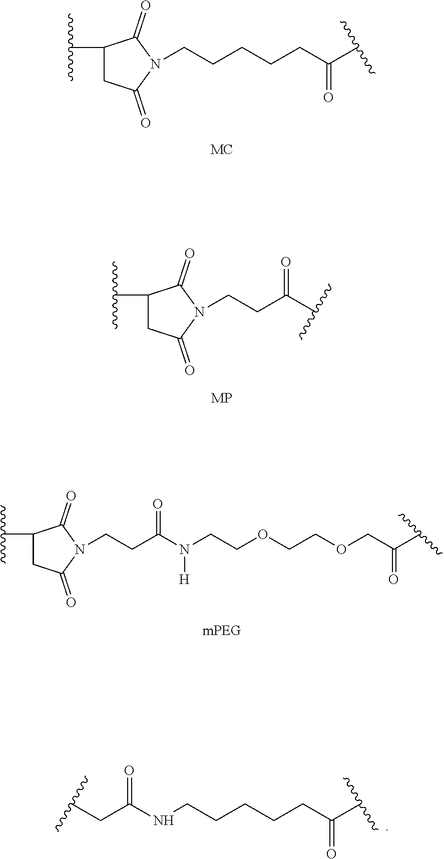 [SMILES: *C(=O)CCCCCN1C(=O)CC(*)C1=O ; *C(=O)CCN1C(=O)CC(*)C1=O ; *C(=O)COCCOCCNC(=O)CCN1C(=O)CC(*)C1=O ; *CC(=O)NCCCCCC(*)=O]

In certain embodiments, the linker is:

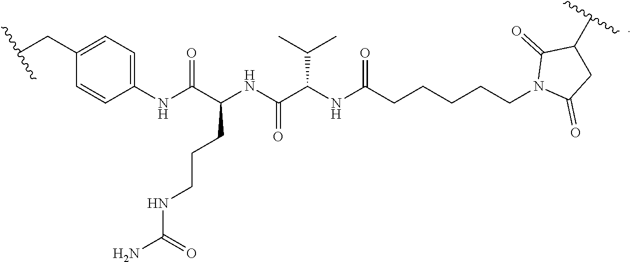 [SMILES: *Cc1ccc(NC(=O)[C@H](CCCNC(N)=O)NC(=O)[C@@H](NC(=O)CCCCCN2C(=O)CC(*)C2=O)C(C)C)cc1]

In certain embodiments, L1 has the structure:

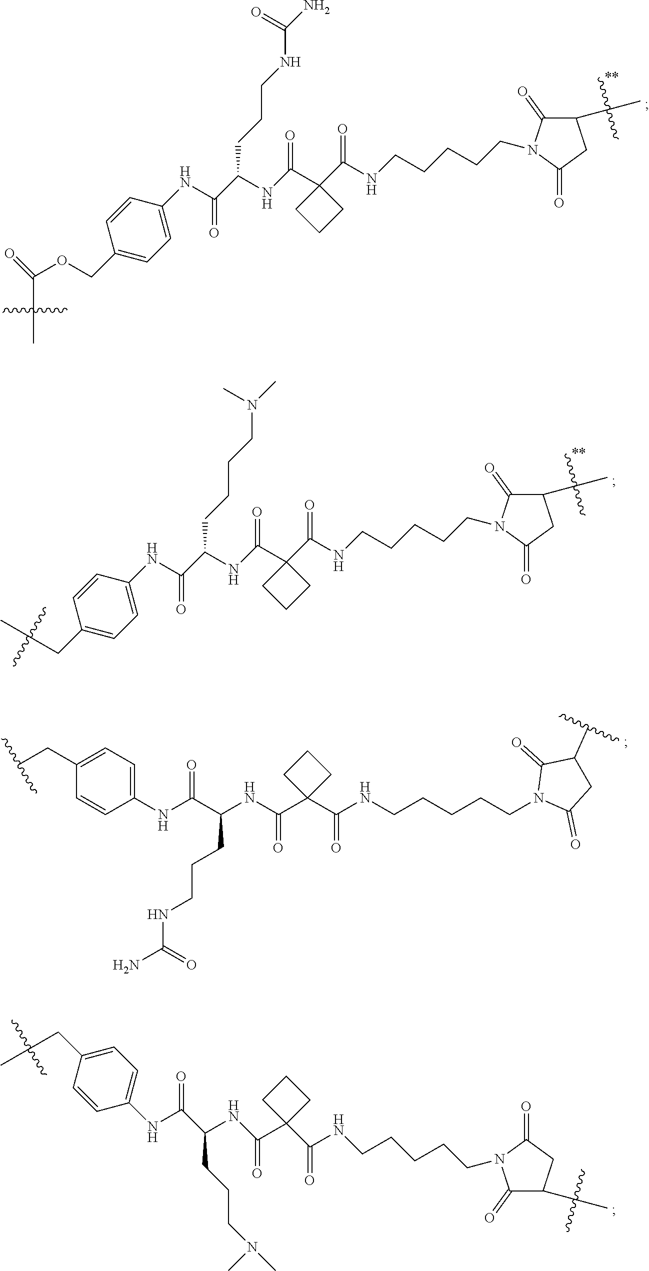 [SMILES: *C(=O)OCc1ccc(NC(=O)[C@H](CCCNC(N)=O)NC(=O)C2(C(=O)NCCCCCN3C(=O)CC(*)C3=O)CCC2)cc1 ; *Cc1ccc(NC(=O)[C@H](CCCCN(C)C)NC(=O)C2(C(=O)NCCCCCN3C(=O)CC(*)C3=O)CCC2)cc1 ; *Cc1ccc(NC(=O)[C@H](CCCNC(N)=O)NC(=O)C2(C(=O)NCCCCCN3C(=O)CC(*)C3=O)CCC2)cc1 ; *Cc1ccc(NC(=O)[C@H](CCCN(C)C)NC(=O)C2(C(=O)NCCCCCN3C(=O)CC(*)C3=O)CCC2)cc1]

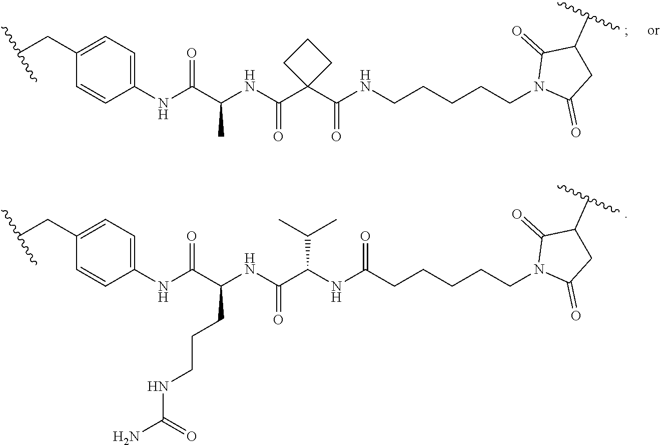 [SMILES: *Cc1ccc(NC(=O)[C@H](C)NC(=O)C2(C(=O)NCCCCCN3C(=O)CC(*)C3=O)CCC2)cc1 ; *Cc1ccc(NC(=O)[C@H](CCCNC(N)=O)NC(=O)[C@@H](NC(=O)CCCCCN2C(=O)CC(*)C2=O)C(C)C)cc1]

### 3. CIDE (“D”)

Useful CIDEs have the general formula described above. In certain embodiments, the CIDE or the Ab-conjugated CIDE exhibits desirable properties such as cell targeting, and protein targeting and degradation. In certain embodiments, the Ab-L1-CIDEs exhibit a DC50 (μg/mL) from 0.0001 to less than about 2.0, or less than about 1.0, or less than about 0.8, or less than about 0.7, or less than about 0.6, or less than about 0.5, or less than about 0.4, or less than about 0.3, or less than about 0.2. In certain embodiments, the Ab-L1-CIDEs exhibit a DCmax of at least 70, 75, 80, 85, 90, 95, 96, 97, 98 or 99. In certain embodiments, the target protein degradation is significantly higher than that of a comparator.

CIDEs include those having the following components.

a. E3 Ubiquitin Ligases Binding Groups (E3LB)

E3 ubiquitin ligases (of which over 600 are known in humans) confer substrate specificity for ubiquitination. There are known ligands which bind to these ligases. As described herein, an E3 ubiquitin ligase binding group is a peptide or small molecule that can bind an E3 ubiquitin ligase that is von Hippel-Lindau (VHL).

A particular E3 ubiquitin ligase is von Hippel-Lindau (VHL) tumor suppressor, the substrate recognition subunit of the E3 ligase complex VCB, which also consists of elongins B and C, Cul2 and Rbxl. The primary substrate of VHL is Hypoxia Inducible Factor 1α (HIF-1α), a transcription factor that upregulates genes such as the pro-angiogenic growth factor VEGF and the red blood cell inducing cytokine erythropoietin in response to low oxygen levels.

In certain embodiments, the subject matter herein is directed to an E3LB portion that comprises a hydroxyproline residue, and is of the formula:

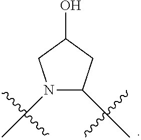 [SMILES: *C1CC(O)CN1*]

In certain embodiments, the the subject matter herein is directed to an E3LB portion that comprises a phosphate moiety.

In certain embodiments, the E3LB comprises:

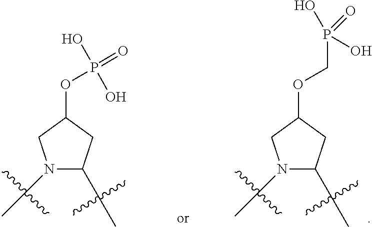 [SMILES: *C1CC(OP(=O)(O)O)CN1* ; *C1CC(OCP(=O)(O)O)CN1*]

In certain embodiments, the EMLB comprises:

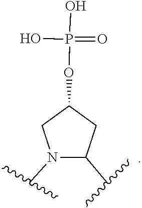 [SMILES: *C1C[C@@H](OP(=O)(O)O)CN1*]

In certain embodiments, the E3LB comprises:

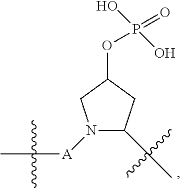

wherein, A is a group covalently bound to L2.

In certain embodiments, the E3LB comprises:

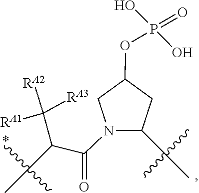

wherein

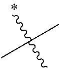

is the attachment point to L2, and
R^{A1}, R^{A2} and R^{A3} are each independently hydrogen, or C₁₋₅ alkyl; or two of R^{A1}, R^{A2} and R^{A3} together with the carbon to which each is attached form a C₁₋₅ cycloalkyl.

In certain embodiments, the E3LB comprises:

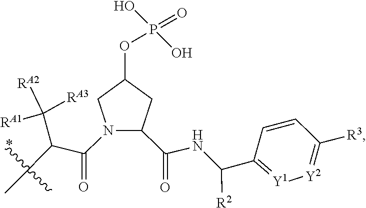

wherein,
R² is hydrogen or C₁₋₅ alkyl;
Y¹ and Y² are each —CH or one of Y¹ and Y² is —CH and the other is N; and
R³ is cyano,

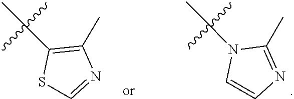

In certain embodiments, the E3LB comprises:

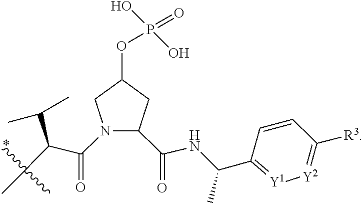

In certain embodiments, the E3LB comprises:

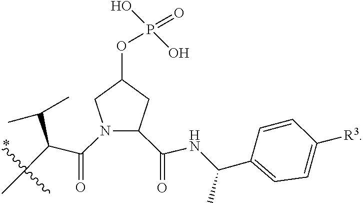

In certain embodiments, E3LB has the structure wherein R₃ is cyano.

In certain embodiments, E3LB has the structure wherein R₃ is

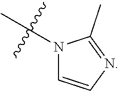 [SMILES: *n1ccnc1C]

In certain embodiments, E3LB has the structure wherein R₃ is

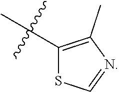 [SMILES: *c1scnc1C]

In certain embodiments, E3LB has the structure wherein R₃ is

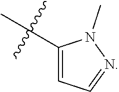 [SMILES: *c1ccnn1C]

In certain embodiments, E3LB has the structure wherein R₂ is hydrogen, methyl, ethyl or propyl.

In certain embodiments, E3LB has the structure wherein R₂ is methyl.

In certain embodiments, E3LB has the structure wherein R₂ is

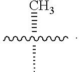

In certain embodiments, the hydroxyproline portion of E3LB has the structure:

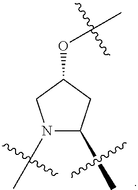

The E3LB portion has at least one terminus with a moiety that is or can be covalently linked to the L2 portion, and at least one terminus with a moiety that is or can be covalently linked to the L1 portion. For example, the E3LB portion terminates in a —NHCOOH moiety that can be covalently linked to the L2 portion through an amide bond.

In any of the aspects or embodiments described herein, the E3LB as described herein may be a pharmaceutically acceptable salt, enantiomer, diastereomer, solvate or polymorph thereof.

b. Protein Binding Group (PB)

The “protein binding group” or “PB” refers to a residue of a small molecule or other compound which is capable of binding to a target protein or other polypeptide target of interest. The PB binds to or otherwise interacts with the target, which places the target in proximity to a ubiquitin ligase such that degradation of the protein or polypeptide by ubiquitin ligase may occur. The PB can be any molecule so long as it is covalently bound to L2 and interacts or binds to a target of interest. Non-limiting examples of small molecule target protein binding moieties include compounds that bind BRM (BRAHMA), Hsp90 inhibitors, kinase inhibitors, MDM2 inhibitors, compounds targeting Human BET Bromodomain-containing proteins, HDAC inhibitors, human lysine methyltransferase inhibitors, angiogenesis inhibitors, immunosuppressive compounds, and compounds targeting the aryl hydrocarbon receptor (AHR), among numerous others. In certain embodiments, the CIDES and conjugated CIDEs described herein are not limited to the type of PB. In certain embodiments, the CIDES and conjugated CIDEs described herein are not limited to the type of PB, wherein the PB is covalently bound to L2; and, the CIDE or conjugated CIDE comprises a phosphate moiety and a L1 peptidomimetic linker.

In certain embodiments, the PB portion of the CIDE is a small molecule that comprises a hydroxyphenyl residue covalently bound to a phosphate moiety through the oxygen of the hydroxyphenyl residue. In this aspect, the PB comprises the formula:

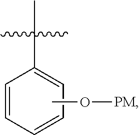

wherein PM is a phosphate moeity. An example of a hydroxyphenyl residue covalently bound to a phosphate moiety is:

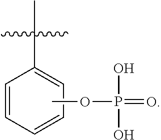

In certain embodiments, the PB is bound in the ortho position:

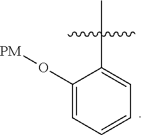

An example of this type of structure is a PB that comprises the formula:

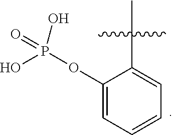 [SMILES: *c1ccccc1OP(=O)(O)O]

In certain embodiments, the PB portion of the CIDE is a small molecule moiety that binds to BRM, including all variants, mutations, splice variants, indels and fusions of BRM. BRM is also known as Subfamily A, Member 2, SMARCA2 and BRAHMA. Such small molecule target protein binding moieties also include pharmaceutically acceptable salts, enantiomers, solvates and polymorphs of these compositions, as well as other small molecules that may target a protein of interest. The CIDEs described herein can comprise any residue of a known BRM binding compound, binding compounds including those disclosed in WO2019/195201, herein incorporated by reference in its entirety.

In certain embodiments, the BRM binding compound is a compound of Formula I:

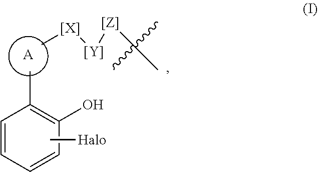

or a stereoisomer or tautomer thereof, or a pharmaceutically acceptable salt of any of the foregoing, wherein:
\textcircled{A} is selected from the group consisting of:

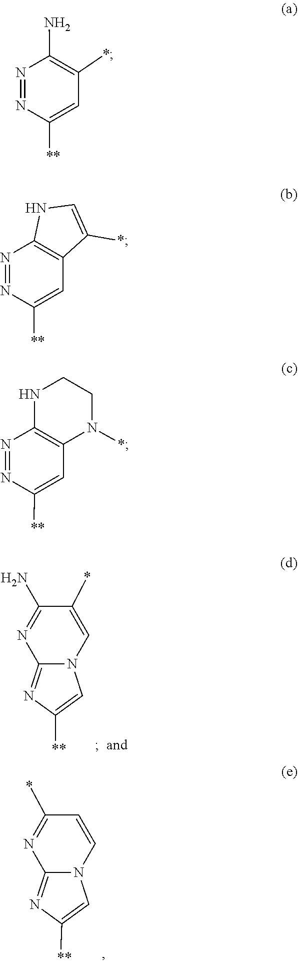

wherein, for (a)-(e), * denotes the point of attachment to [X], or, if [X] is absent, * denotes the point of attachment to [Y], and ** denotes the point of attachment to the phenyl ring; and wherein:
(i) [X] is 3-15 membered heterocyclyl or 5-20 membered heteroaryl, provided that, when \textcircled{A} is (a), then [X] is not

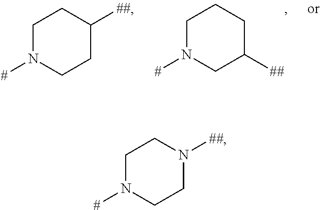

wherein #denotes the point of attachment to and ##denotes the point of attachment to L2,
[Y] is absent, and
[Z] is absent; or
(ii) [X] is 3-15 membered heterocyclyl or 5-20 membered heteroaryl, wherein the 3-15 membered heterocyclyl of [X] is optionally substituted with one or more —OH or C₁₋₆alkyl,
   [Y] is absent, and
[Z] is 3-15 membered heterocyclyl or 5-20 membered heteroaryl, provided that, when \textcircled{A} is (a) and [X] is

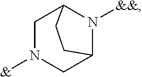

wherein & denotes the point of attachment to \textcircled{A} and && denotes the point of attachment to [Z], then [Z] is not

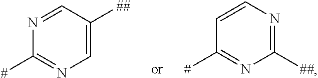

wherein #denotes the point of attachment to [X] and ##denotes the point of attachment to L2; or
(iii) [X] is 3-15 membered heterocyclyl or 5-20 membered heteroaryl,
[Y] is methylene, wherein the methylene of [Y] is optionally substituted with one or more methyl group, and
   [Z] is 3-15 membered heterocyclyl; or
(iv) [X] is absent,
[Y] is ethenylene, wherein the ethenylene of [Y] is optionally substituted with one or more halo, and
   [Z] is 5-20 membered heteroaryl,
provided that \textcircled{A} is (a), (b), (d), or (e); or
(v) [X] is absent,
   [Y] is ethynylene, and
   [Z] is 5-20 membered heteroaryl,
provided that \textcircled{A} is (a), (b), (d), or (e); or
(vi) [X] is absent,
   [Y] is cyclopropyl or cyclobutyl, and
   [Z] is 5-20 membered heteroaryl,
provided that \textcircled{A} is (a), (b), (d), or (e).

In certain embodiments, the BRM binding compound is a compound of formula (I-A):

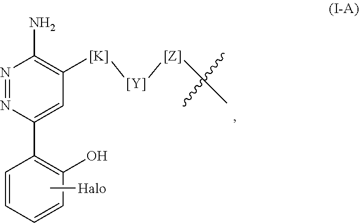

or a stereoisomer or tautomer thereof, or a pharmaceutically acceptable salt of any of the foregoing, wherein [X], [Y] and [Z] are as defined above for a compound of formula (I).

In certain embodiments, the BRM binding compound is a compound of formula (I-B):

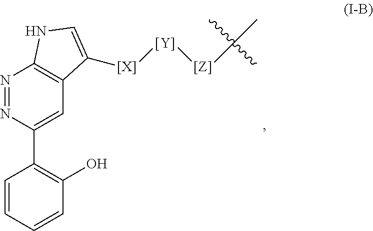

or a stereoisomer or tautomer thereof, or a pharmaceutically acceptable salt of any of the foregoing, wherein [X], [Y] and [Z] are as defined above for a compound of formula (I).

In certain embodiments, the BRM binding compound is a compound of formula (I-C):

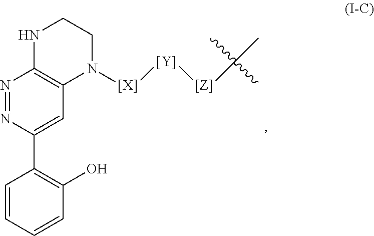

or a stereoisomer or tautomer thereof, or a pharmaceutically acceptable salt of any of the foregoing, wherein [X], [Y] and [Z] are as defined above for a compound of formula (I).

In certain embodiments, the BRM binding compound is a compound of formula (I-D):

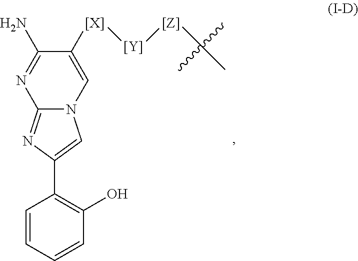

or a stereoisomer or tautomer thereof, or a pharmaceutically acceptable salt of any of the foregoing, wherein [X], [Y] and [Z] are as defined above for a compound of formula (I).

In certain embodiments, the BRM binding compound is a compound of formula (I-E):

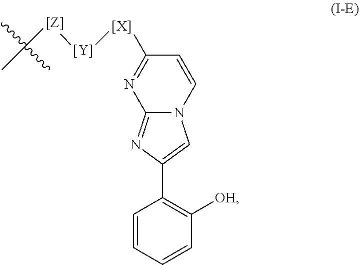

or a stereoisomer or tautomer thereof, or a pharmaceutically acceptable salt of any of the foregoing, wherein [X], [Y] and [Z] are as defined above for a compound of formula (I).

In certain embodiments, the PB (BRM) portion of the CIDE has the structure:

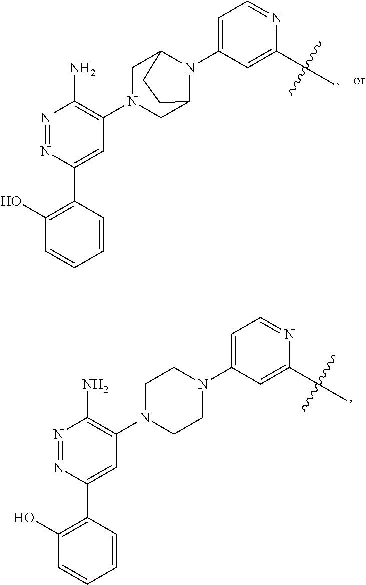

wherein,

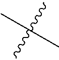

is the point of covalent attachment to L2.
   c. Linker L2

The E3LB and PB portions of CIDEs as described herein can be connected with linker (L2, Linker L2, Linker-2). In certain embodiments, the Linker L2 is covalently bound to the E3LB portion and covalently bound to the PB portion, thus making up the CIDE.

In certain embodiments, the L2 portion can be selected from linkers disclosed in WO2019/195201, herein incorporated by reference in its entirety.

Although the E3LB group and PB group may be covalently linked to the linker group through any group which is appropriate and stable to the chemistry of the linker, in certain aspects, the L2 is independently covalently bonded to the E3LB group and the PB group through an amide, ester, thioester, keto group, carbamate (urethane) or ether, each of which groups may be inserted anywhere on the E3LB group and PB group to allow binding of the E3LB group to the ubiquitin ligase and the PB group to the target protein such as BRM to be degraded. In other words, as shown herein, the linker can be designed and connected to E3LB and PB to modulate the binding of E3LB and PB to their respective binding partners.

In certain embodiments, L2 is a linker covalently bound to E3LB and PB, the L2 having the formula.

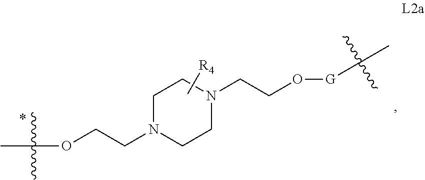

wherein,
R₄ is hydrogen or methyl,

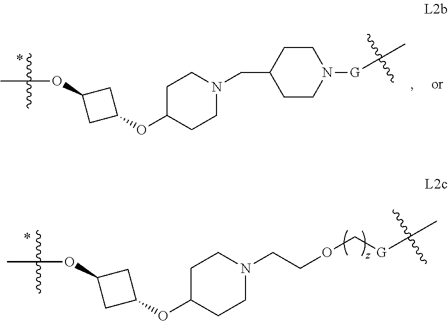

wherein,
z is one or zero,
G is

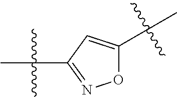 [SMILES: *c1cc(*)on1]

or —C(O)NH—; and,

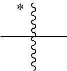

is the point of attachment to the CIDE, such as directly bound to the PB.

In certain embodiments of L2a, R₄ is hydrogen.

In certain embodiments of L2a, R₄ is methyl.

In certain embodiments of L2a, R₄ is a methyl, such that the methyl is oriented relative to the piperazine to which it is attached as follows:

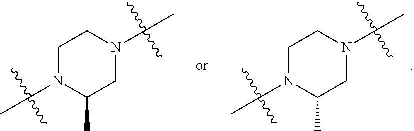

In certain embodiments of L2c, z is zero.

In certain embodiments of L2c, z is one.

Referring now to an Ab-CIDE, an Ab-CIDE can comprise a single antibody where the single antibody can have more than one CIDE, each CIDE covalently linked to the antibody through a linker L1. The “CIDE loading” is the average number of CIDE moieties per antibody. CIDE loading may range from 1 to 16 CIDE (D) per antibody (Ab). That is, in the Ab-CIDE formula, Ab-(L1-D)ⱼ, j has a value from about 1 to about 16, from about 1 to about 8, from about 1 to about 5, from about 1 to about 4, or from about 1 to about 3. Each CIDE covalently linked to the antibody through linker L1 can be the same or different CIDE and can have a linker of the same type or different type as any other L1 covalently linked to the antibody. In certain embodiments, Ab is a cysteine engineered antibody and j is about 1, 2, 3, 4, 5, 6, 7, 8, 9, 10, 11, 12, 13, 14, 15 or 16.

The average number of CIDEs per antibody in preparations of Ab-CIDEs from conjugation reactions may be characterized by conventional means such as mass spectrometry, ELISA assay, electrophoresis, and HPLC. The quantitative distribution of Ab-CIDEs in terms of j may also be determined. By ELISA, the averaged value of j in a particular preparation of Ab-CIDE may be determined (Hamblett et al (2004) Clin. Cancer Res. 10:7063-7070; Sanderson et al (2005) Clin. Cancer Res. 11:843-852). However, the distribution of the value of p is not discernible by the antibody-antigen binding and detection limitation of ELISA. Also, ELISA assay for detection of Ab-CIDEs does not determine where the CIDE moieties are attached to the antibody, such as the heavy chain or light chain fragments, or the particular amino acid residues. In some instances, separation, purification, and characterization of homogeneous Ab-CIDEs where j is a certain value from Ab-CIDEs with other CIDE loadings may be achieved by means such as reverse phase HPLC or electrophoresis.

For some Ab-CIDEs, j may be limited by the number of attachment sites on the antibody. For example, an antibody may have only one or several cysteine thiol groups, or may have only one or several sufficiently reactive thiol groups through which a linker may be attached. Another reactive site on an Ab to connect L1-Ds are the amine functional group of lysine residues. Values of j include values from about 1 to about 16, from about 1 to about 8, from about 1 to about 5, from about 1 about 4, from about 1 to about 3, and where j is equal to 2. In some embodiments, the subject matter described herein is directed to any the Ab-CIDEs, wherein j is about 1, 2, 3, 4, 5, 6, 7, or 8.

Generally, fewer than the theoretical maximum of CIDE moieties is conjugated to an antibody during a conjugation reaction. An antibody may contain, for example, many lysine residues that do not react with the linker L1-CIDE group (L1-D) or linker reagent. Only the most reactive lysine groups may react with an amine-reactive linker reagent. Also, only the most reactive cysteine thiol groups may react with a thiol-reactive linker reagent or linker L1-CIDE group. Generally, antibodies do not contain many, if any, free and reactive cysteine thiol groups which may be linked to a CIDE moiety. Most cysteine thiol residues in the antibodies of the compounds exist as disulfide bridges and must be reduced with a reducing agent such as dithiothreitol (DTT) or TCEP, under partial or total reducing conditions. However, the CIDE loading (CIDE/antibody ratio, “DAR”) of a DAR may be controlled in several different manners, including: (i) limiting the molar excess of linker L1-CIDE group or linker reagent relative to antibody, (ii) limiting the conjugation reaction time or temperature, and (iii) partial or limiting reductive conditions for cysteine thiol modification.

### III. L1-CIDE Compounds

The CIDEs described herein can be covalently linked to a linker L1 to prepare L1-CIDE groups. These compounds have the following general formula:

L1-D,

wherein D is a CIDE having the structure E3LB-L2-PB; wherein, E3LB is an E3 ligase binding group covalently bound to L2; L2 is a linker covalently bound to E3LB and PB; PB is a target protein binding group covalently bound to L2; and L1 is a peptidomimetic linker covalently bound to D. Useful groups for each of these components are as described above. In certain embodiments, the CIDE is phosphorylated.

In certain embodiments, L1 is as described elsewhere herein, including a peptidomimetic linker. In these embodiments, the L1-CIDE has the following formula:

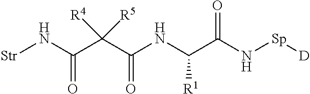

wherein
Str is a stretcher unit;
Sp is a bond or a spacer unit covalently attached to D, i.e., a CIDE moiety;
R¹ is C₁-C₁₀alkyl, (C₁-C₁₀alkyl)NHC(NH)NH₂ or (C₁-C₁₀alkyl)NHC(O)NH₂;
R⁴ and R⁵ are each independently C₁-C₁₀alkyl, arylalkyl, heteroarylalkyl, (C₁-C₁₀alkyl)OCH₂—,
or R⁴ and R⁵ may form a C₃-C₇cycloalkyl ring;
D is a CIDE moiety.

A L1-CIDE compound can be represented by the following formula:

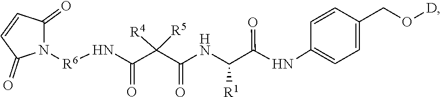

wherein R₆ is C₁-C₁₀alkylene; R⁴ and R⁵ together form a C₃-C₇cycloalkyl ring, and D is a CIDE moiety.

A L1-CIDE compound can be represented by the following formula:

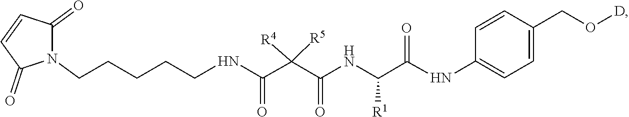

wherein R¹, R⁴ and R⁵ are as described elsewhere herein, and D is a CIDE moiety.

An L1-CIDE compound can be represented by the following formula:

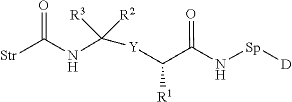

wherein
Str is a stretcher unit;
Sp is an optional spacer unit covalently attached to D, i.e., a CIDE moiety;
Y is heteroaryl, aryl, —C(O)C₁-C₆alkylene, C₁-C₆alkylene-NH₂, C₁-C₆alkylene-NH—CH₃, C₁-C₆alkylene-N—(CH₃)₂, C₁-C₆alkenyl or C₁-C₆alkylenyl;
R¹ is C₁-C₁₀alkyl, (C₁-C₁₀alkyl)NHC(NH)NH₂ or (C₁-C₁₀alkyl)NHC(O)NH₂;
R³ and R² are each independently H, C₁-C₁₀alkyl, arylalkyl or heteroarylalkyl, or R³ and R² together may form a C₃-C₇cycloalkyl; and
D is a CIDE moiety.

A L1-CIDE compound can be represented by the following formula:

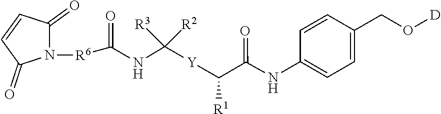

wherein, R⁶ is C₁-C₁₀alkylene, and R¹, R² and R³ are as described elsewhere herein, and D is a CIDE moiety

A L1-CIDE compound can be represented by the following formula:

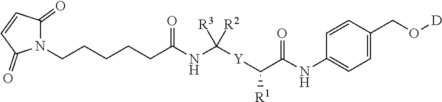

wherein R¹, R² and R³ are as described elsewhere herein, and D is a CIDE moiety.

In any of the above L1-CIDE compounds, Str can have the following formula:

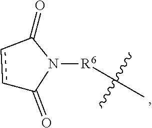

wherein R⁶ is selected from the group consisting of C₁-C₁₀alkylene, C₃-C₈cycloalkyl, O—(C₁-C₈alkylene), and C₁-C₁₀alkylene-C(O)N(R^{a})—C₂-C₆alkylene, where each alkylene may be substituted by one to five substituents selected from the group consisting of halo, trifluoromethyl, difluoromethyl, amino, alkylamino, cyano, sulfonyl, sulfonamide, sulfoxide, hydroxy, alkoxy, ester, carboxylic acid, alkylthio, C₃-C₈cycloalkyl, C₄-C₇heterocycloalkyl aryl, arylalkyl, heteroarylalkyl and heteroaryl; each R^{a} is independently H or C₁-C₆alkyl; Sp is —Ar—R^{b}—, wherein Ar is aryl or heteroaryl, R^{b} is (C₁-C₁₀alkylene)O—.

In certain L1-CIDE compounds, R⁶ is C₁-C₁₀alkylene, Sp is —Ar—R^{b}—, wherein Ar is aryl R^{b} is (C₁-C₆alkylene)O—; or R₆ is —(CH₂)_{q} is 1-10;

In any of the above L1-CIDE compounds, Str can have the following formula:

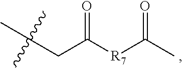

wherein,

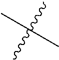

indicates attachment to a moiety capable of conjugating to an antibody, R⁷ is selected from C₁-C₁₀alkylene, C₁-C₁₀alkylene-O, N(R^{c})—(C₂-C₆ alkylene)-N(R^{c}) and N(R^{c})—(C₂-C₆alkylene); where each R^{c} is independently H or C₁-C₆ alkyl; Sp is —Ar R^{b} wherein Ar is aryl or heteroaryl, R^{b} is (C₁-C₁₀ alkylene)O—; or wherein R⁶ is C₁-C₁₀ alkylene, Sp is —Ar—R^{b}—, wherein Ar is aryl R^{b} is (C₁-C₆ alkylene)O—.

A L1-CIDE can have the following formulae, wherein in each instance, D is a CIDE moiety:

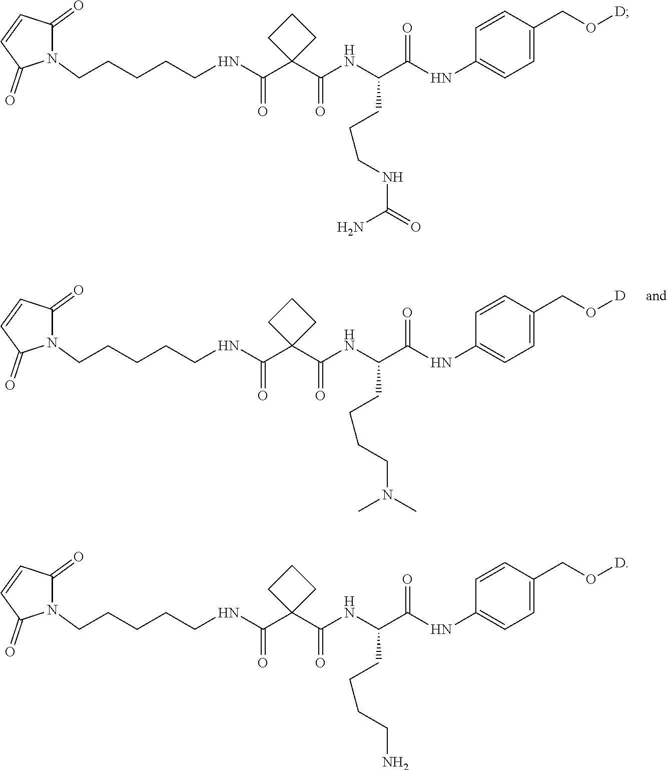

Ab-CIDEs can include any combination of PB, E3LB, Ab, L1 and L2; and those of skill in the art would understand that the L1 and L2 points of attachment can vary, so long as the CIDE, L1-CIDE or Ab-L1-CIDE comprises a phosphate group covalently bound to PB or E3LB and a peptidomimetic linker, when L1 is present. Further, portions of the linkers, such as -Str-(PM)-Sp can be interchanged. Additionally, portions of linkers L1 can be interchanged.

Referring now to an Ab-CIDE and a L1-CIDE compound, as described herein, these can exist in solid or liquid form. In the solid state, it may exist in crystalline or noncrystalline form, or as a mixture thereof. The skilled artisan will appreciate that pharmaceutically acceptable solvates may be formed for crystalline or non-crystalline compounds. In crystalline solvates, solvent molecules are incorporated into the crystalline lattice during crystallization. Solvates may involve non-aqueous solvents such as, but not limited to, ethanol, isopropanol, DMSO, acetic acid, ethanolamine, or ethyl acetate, or they may involve water as the solvent that is incorporated into the crystalline lattice. Solvates wherein water is the solvent incorporated into the crystalline lattice are typically referred to as “hydrates.” Hydrates include stoichiometric hydrates as well as compositions containing variable amounts of water. The subject matter described herein includes all such solvates.

The skilled artisan will further appreciate that certain compounds and Ab-CIDEs described herein that exist in crystalline form, including the various solvates thereof, may exhibit polymorphism (i.e. the capacity to occur in different crystalline structures). These different crystalline forms are typically known as “polymorphs.” The subject matter disclosed herein includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

Compounds and Ab-CIDEs described herein or a salt thereof may exist in stereoisomeric forms (e.g., it contains one or more asymmetric carbon atoms). The individual stereoisomers (enantiomers and diastereomers) and mixtures of these are included within the scope of the subject matter disclosed herein. Likewise, it is understood that a compound or salt of Formula (I) may exist in tautomeric forms other than that shown in the formula and these are also included within the scope of the subject matter disclosed herein. It is to be understood that the subject matter disclosed herein includes all combinations and subsets of the particular groups described herein. The scope of the subject matter disclosed herein includes mixtures of stereoisomers as well as purified enantiomers or enantiomerically/diastereomerically enriched mixtures. It is to be understood that the subject matter disclosed herein includes all combinations and subsets of the particular groups defined hereinabove.

The subject matter disclosed herein also includes isotopically-labelled forms of the compounds described herein, but for the fact that one or more atoms are replaced by an atom having an atomic mass or mass number different from the atomic mass or mass number usually found in nature. Examples of isotopes that can be incorporated into compounds described herein and pharmaceutically acceptable salts thereof include isotopes of hydrogen, carbon, nitrogen, oxygen, phosphorous, sulphur, fluorine, iodine, and chlorine, such as ²H, ³H, ¹¹C, ¹³C, ¹⁵N, ¹⁷O, ¹⁸O, ³¹P, ³²P, ³⁵S, ¹⁸F, ³⁶Cl, ¹²³I and ¹²⁵I.

Compounds and Ab-CIDEs as disclosed herein and pharmaceutically acceptable salts thereof that contain the aforementioned isotopes and/or other isotopes of other atoms are within the scope of the subject matter disclosed herein. Isotopically-labelled compounds are disclosed herein, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are commonly used for their ease of preparation and detectability. ¹¹C and ¹⁸F isotopes are useful in PET (positron emission tomography), and ¹²⁵I isotopes are useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds can generally be prepared by carrying out the procedures disclosed in the Schemes and/or in the Examples below, by substituting a readily available isotopically labelled reagent for a non-isotopically labelled reagent.

In certain embodiments, the subject matter described herein includes the following L1-CIDEs:

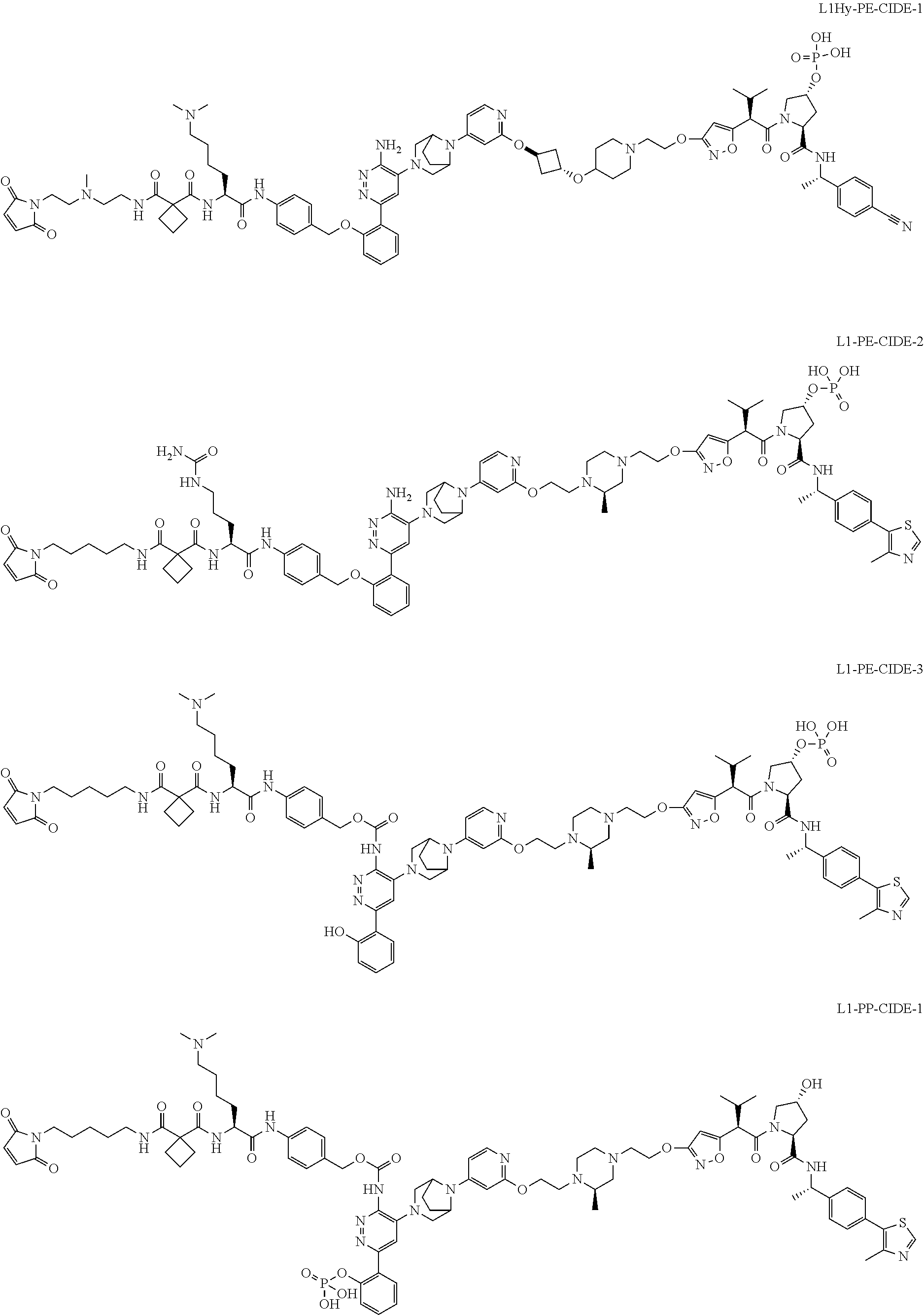

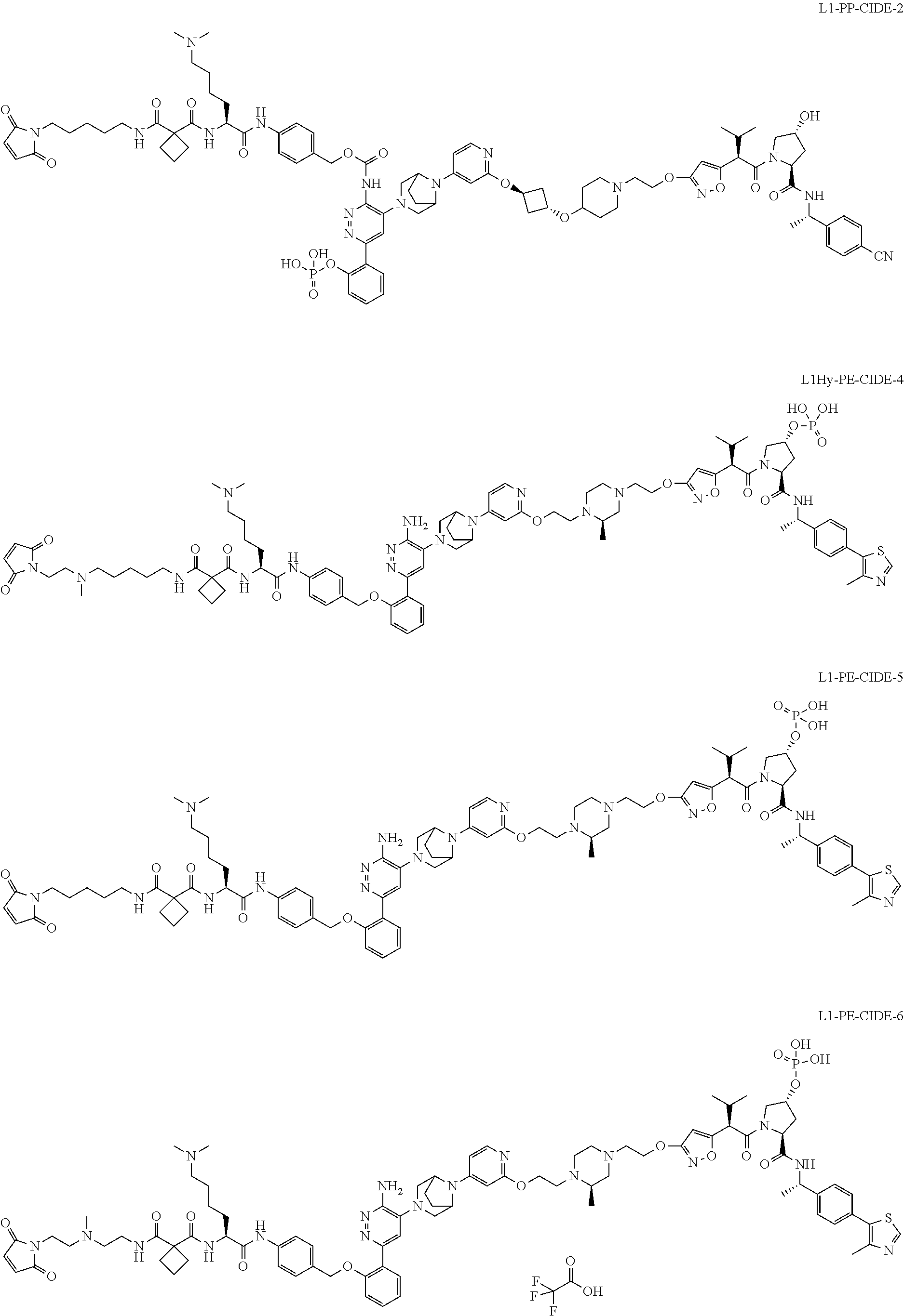

In embodiments, at least one of L1Hy-PE-CIDE-1, L1-PE-CIDE-2, L1-PE-CIDE-3, L1Hy-PE-CIDE-4, L1-PE-CIDE-5, L1Hy-PE-CIDE-6, L1-PP-CIDE-1 and L1-PP-CIDE-2 is conjugated to an antibody. In embodiments, at least one L1Hy-PE-CIDE-1 is conjugated to an antibody. In embodiments, at least one L1-PE-CIDE-2 is conjugated to an antibody. In embodiments, at least one L1-PE-CIDE-3 is conjugated to an antibody. In embodiments, at least one L1Hy-PE-CIDE-4 is conjugated to an antibody. In embodiments, at least one L1-PE-CIDE-5 is conjugated to an antibody. In embodiments, at least one L1Hy-PE-CIDE-6 is conjugated to an antibody. In embodiments, at least one L1-PP-CIDE-1 is conjugated to an antibody. In embodiments, at least one L1-PP-CIDE-2 is conjugated to an antibody.

The subject matter disclosed herein include the following non-limiting embodiments:
1. A conjugate having the chemical structure:

Ab-(L1-D)ⱼ,
wherein,
D is a CIDE having the structure:

E3LB-L2-PB,
wherein,
   E3LB is an E3 ligase binding (E3LB) ligand covalently bound to L2,
   wherein the E3 ligase is von Hippel-Lindau (VHL) tumor suppressor protein;
   L2 is a linker covalently bound to E3LB and PB;
   PB is a protein binding group covalently bound to L1 and to L2;
   wherein, at least one phosphate moiety is covalently bound to D;
Ab is an antibody covalently bound to L1;
L1 is a linker covalently bound to Ab and D; and
j has a value of from about 1 to about 16.
2. The conjugate of embodiment 1, wherein one phosphate moiety is covalently bound to D at either the E3LB or the PB.
3. The conjugate of embodiment 1 or 2, wherein L1 is a peptidomimetic linker covalently bound to Ab and to the PB of D.
4. The conjugate of embodiment 1 or 2, wherein L1 is a peptidomimetic linker covalently bound to Ab and to the E3LB of D.
5. The conjugate of embodiment 1, 2, 3 or 4, wherein L1 is selected from the group consisting of:
i)

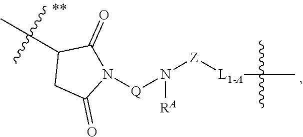

wherein

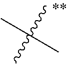

indicates the point of attachment to Ab;
Z is —(CH₂)ₚ— or —CH₂—(CH₂—O—CH₂)ₚ—CH₂—, wherein p is 1, 2, 3, 4, 5 or 6;
R^{A} is hydrogen, C₁₋₆ alkyl, or —(CH₂)ᵥ-aryl, wherein v is 0 or 1 (such as phenyl or benzyl);
Q is selected from the group consisting of:
a)

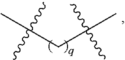

wherein, q is 1, 2, 3 or 4; and
b)

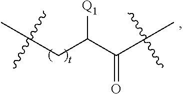

wherein t is 0, 1, 2, 3 or 4;
   Q¹ is hydrogen,

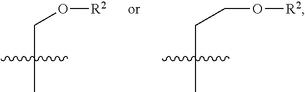

wherein R² is hydrogen, halo(C₁₋₆)alkyl or C₁₋₆ alkyl; and
L_{1-A} is:

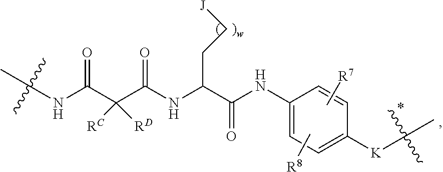

wherein

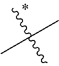

indicates the attachment point to D; and
ii)

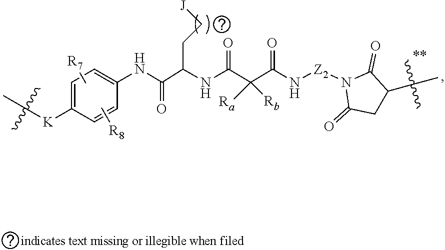

wherein

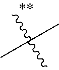

indicates the attachment point to Ab;
Z₂ is a C₁₋₁₂ alkylene or —[CH₂]_{g}—O—[CH₂]ₕ—, wherein g and h are each independently 0, 1 or 2;
w is 0, 1, 2, 3, 4 or 5;
J is selected from the group consisting of C₁₋₅ alkyl, —N(Rₓ)(R_{y}), —C(O)NH₂, —NH—C(O)—NH₂, and —NHC(═NH)NH₂, wherein, Rₓ and R_{y} are each independently selected from hydrogen and C₁₋₃alkyl; K is selected from the group consisting of C₁₋₃alkylene, —CH(R)—, —C(O)—, —C(O)—O—CH(R)—, —CH₂—O—C(O)—, —CH₂—O—C(O)—NH—CH₂—, and —CH₂—O—C(O)—R—[CH₂]q-O—, wherein R is hydrogen, C₁₋₃alkyl, N(Rₓ)(R_{y}), —O—N(Rₓ)(R_{y}) or C(O)—N(Rₓ)(R_{y}), wherein q is 0, 1, 2, or 3, and wherein Rₓ and R_{y} are each independently selected from hydrogen and C₁₋₃alkyl, or Rₓ and R_{y} together with the nitrogen to which each is attached form an optionally substituted 5- to 7-member heterocyclyl;
Rₐ, R_{b}, R^{C} and R^{D} are each independently selected from hydrogen and C₁₋₃alkyl or Rₐ and R_{b} together with the carbon to which each is attached form an optionally substituted C₃₋₆cycloalkyl; and
R₇ and R₈ are each independently hydrogen, halo, C₁₋₅ alkyl, C₁₋₅ alkoxy or hydroxyl.
6. The conjugate of embodiment 5, wherein K of L1 is selected from the group consisting of:

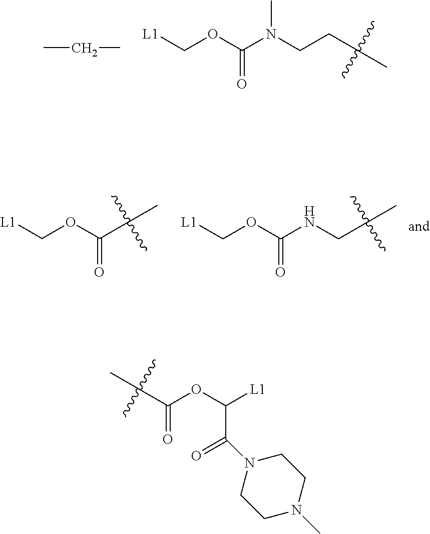

7. The conjugate of embodiment 1, 2, 3, 4 or 5, wherein L1 is selected from the group consisting of:

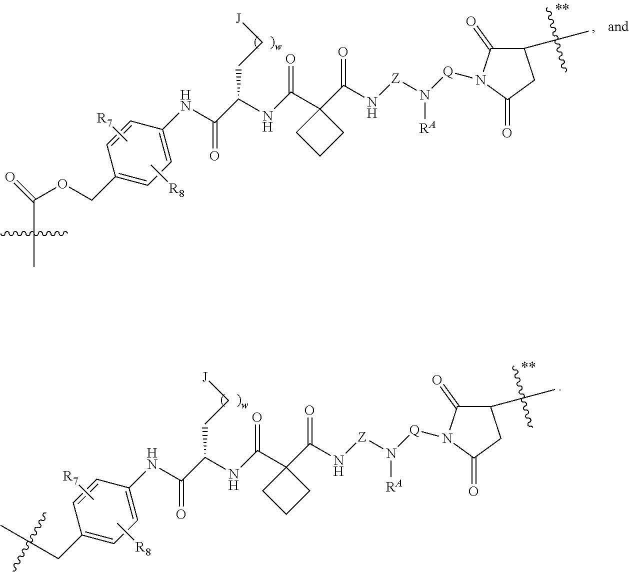

8. The conjugate of embodiment 7, wherein R₇ and R₈ are each hydrogen.
9. The conjugate of embodiment 8, wherein Z is —(CH₂)ₚ—.
10. The conjugate of embodiment 9, wherein w is 2 or 3, and J is —N(CH₃)₂ or —NH(CO)NH.
11. The conjugate of embodiment 10, wherein Q is —CH₂—CH₂—.
12. The conjugate of embodiment 1, 2, 3, 4 or 5, wherein L1 is selected from the group consisting of:

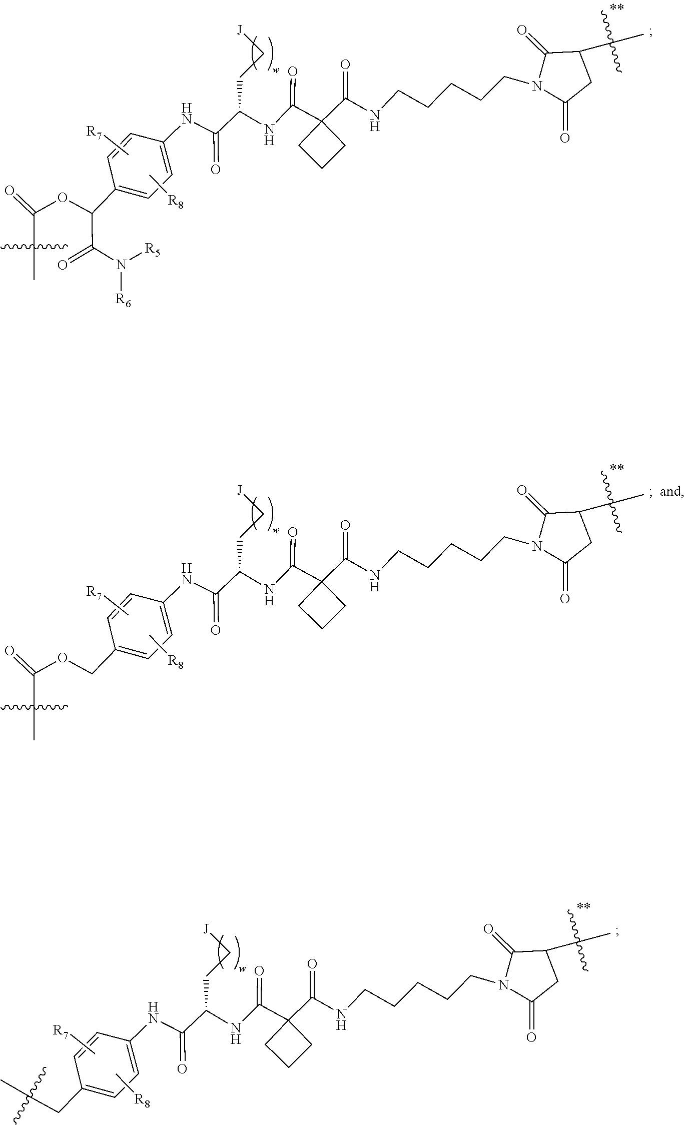

wherein,
R₅ and R₆ are independently hydrogen or C₁₋₅ alkyl; or R₅ and R₆ together with the nitrogen to which each is attached form an optionally substituted 5- to 7-member heterocyclyl.
13. The conjugate of embodiment 12, wherein L1 is selected from the group consisting of:

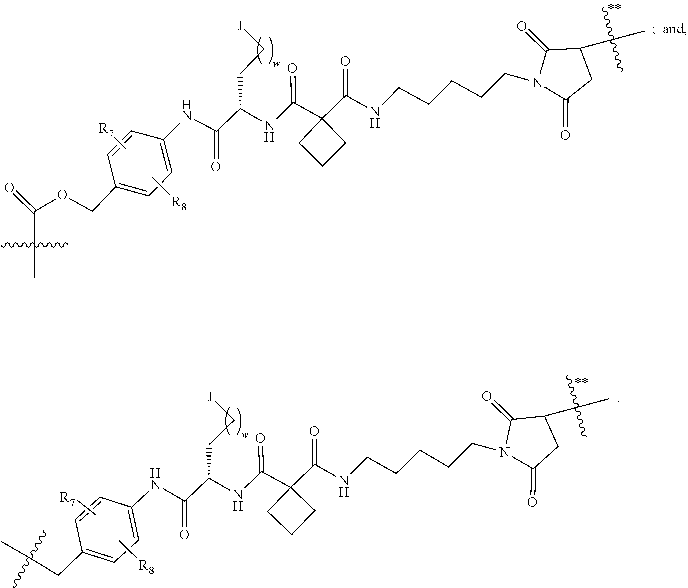

14. The conjugate of embodiment 13, wherein J is —NH—C(O)—NH₂ or —N(CH₃)₂.
15. The conjugate of embodiment 14, wherein w is 2 and J is —NH—C(O)—NH₂; or w is 3 and J is N(CH₃)₂.
16. The conjugate of embodiment 15, wherein L1 has the structure:

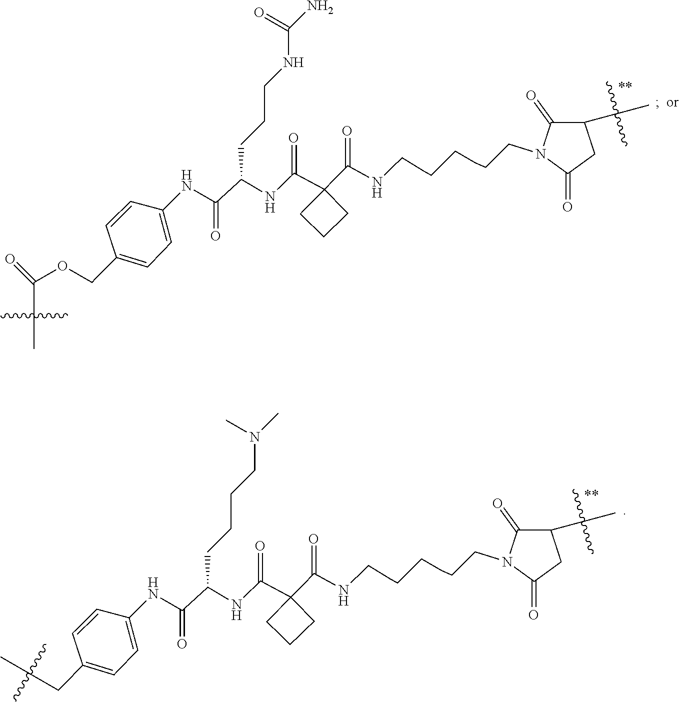 [SMILES: *C(=O)OCc1ccc(NC(=O)[C@H](CCCNC(N)=O)NC(=O)C2(C(=O)NCCCCCCCN3C(=O)CC(*)C3=O)CCC2)cc1 ; *Cc1ccc(NC(=O)[C@H](CCCCN(C)C)NC(=O)C2(C(=O)NCCCCCCCN3C(=O)CC(*)C3=O)CCC2)cc1]

17. The conjugate of any one of embodiments 1-16, wherein the phosphate moiety is selected from the group consisting of: —(P═O)(OH)₂, —CH₂O(P═O)(OH)₂, —(P═O)(OH)O(P═O)(OH)₂ and —CH₂—O(P═O)(OH)O(P═O)(OH)₂.
18. The conjugate of any one of embodiments 1-17, wherein the E3LB comprises a hydroxyproline residue.
19. The conjugate of embodiment 18, wherein the phosphate moiety is covalently bound to E3LB through the oxygen of the hydroxyproline residue.
20. The conjugate of embodiment 19, wherein the E3LB comprises:

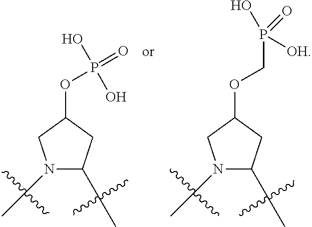 [SMILES: *C1CC(OP(=O)(O)O)CN1* ; *C1CC(OCP(=O)(O)O)CN1*]

21. The conjugate of embodiment 20, wherein the E3LB comprises:

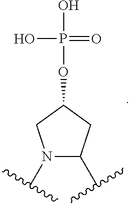 [SMILES: *C1C[C@@H](OP(=O)(O)O)CN1*]

22. The conjugate of embodiment 21, wherein the L1-D to which the antibody is conjugated is selected from the group consisting of:

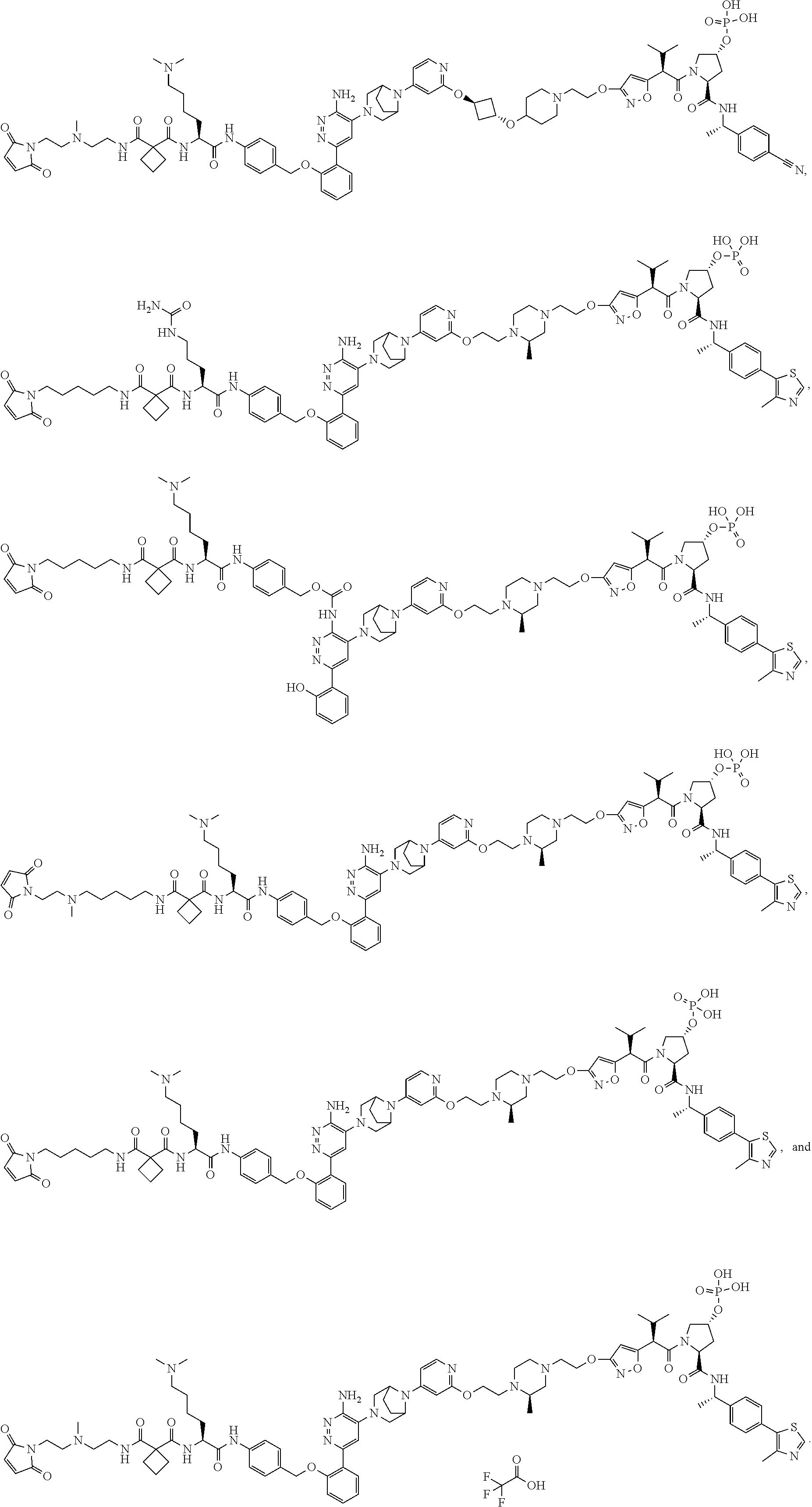

23. The conjugate of any one of embodiments 1-22, wherein the PB comprises a hydroxyphenyl moiety.
24. The conjugate of embodiment 23, wherein the phosphate moiety is covalently bound to PB through the oxygen of the hydroxyphenyl moiety.
25. The conjugate of embodiment 24, wherein the PB is a BRM ligand.
26. The conjugate of embodiment 25, wherein the L1-D to which the antibody is conjugated is selected from the group consisting of:

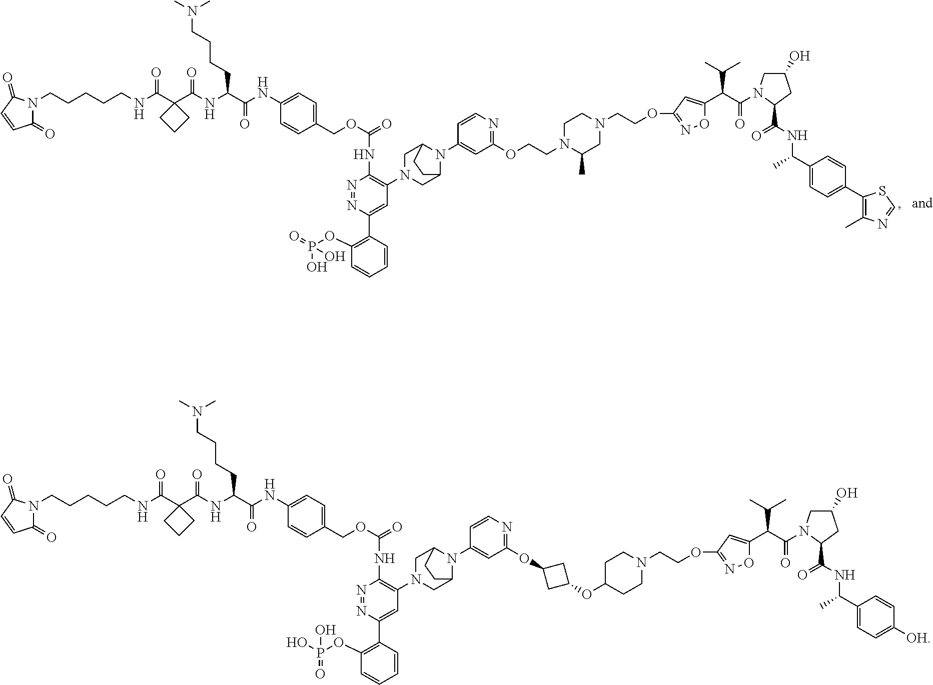 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7OP(=O)(O)O)nnc5NC(=O)OCc5ccc(NC(=O)[C@H](CCCCN(C)C)NC(=O)C7(C(=O)NCCCCCN8C(=O)C=CC8=O)CCC7)cc5)C6)ccn4)[C@H](C)C3)no2)C(C)C)cc1 ; CC(C)[C@@H](C(=O)N1C[C@H](O)C[C@H]1C(=O)N[C@@H](C)c1ccc(O)cc1)c1cc(OCCN2CCC(O[C@H]3C[C@H](Oc4cc(N5C6CCC5CN(c5cc(-c7ccccc7OP(=O)(O)O)nnc5NC(=O)OCc5ccc(NC(=O)[C@H](CCCCN(C)C)NC(=O)C7(C(=O)NCCCCCN8C(=O)C=CC8=O)CCC7)cc5)C6)ccn4)C3)CC2)no1]

27. A pharmaceutical composition comprising a conjugate of any one of embodiments 1-26 and one or more pharmaceutically acceptable excipients.
28. A method of treating a disease in a human in need thereof, comprising administering to said human an effective amount of a conjugate of any one of embodiments 1-26 or a composition of embodiment 27; or, A conjugate of any one of embodiments 1-26 (or a composition thereof) or a composition of embodiment 27 for use in treating a disease in a human in need thereof, or, Use of a conjugate of any one of embodiments 1-26 (or a composition thereof) or a composition of embodiment 27 for the manufacture of a medicament for the treatment of a disease in a human in need thereof.
29. The method of embodiment 28, wherein said disease is cancer.
30. The method of embodiment 29, wherein said cancer is BRM-dependent.
31. The method of embodiment 30, wherein said cancer is non-small cell lung cancer.
32. A method of reducing the level of a target protein in a subject comprising, administering a conjugate of any one of embodiments 1-26 or a composition of embodiment 27 to said subject, wherein said PB portion binds said target protein, wherein ubiquitin ligase effects degradation of said bound target protein, wherein the level of said target protein is reduced.

In embodiments, the disclosed compound has the chemical structure:

L1-D,
wherein,
L1 is a peptidomimetic linker covalently bound to D; and
D is a CIDE having the structure:

E3LB-L2-PB,
wherein,
E3LB is an E3 ligase binding (E3LB) ligand covalently bound to L2, wherein the E3 ligase is von Hippel-Lindau (VHL) tumor suppressor protein, and wherein E3LB comprises a hydroxyproline residue having a phosphate moiety covalently bound to E3LB through the oxygen of the hydroxyproline residue,
PB is a protein binding group covalently bound to L1 and to L2, and
L2 is a linker covalently bound to E3LB and PB.

In embodiments, the disclosed compound has the chemical structure:

L1-D,
wherein,
L1 is a peptidomimetic linker covalently bound to D; and
D is a CIDE having the structure:
E3LB-L2-PB,
wherein
E3LB is an E3 ligase binding (E3LB) ligand covalently bound to L2, wherein the E3 ligase is von Hippel-Lindau (VHL) tumor suppressor protein,
PB is a protein binding group covalently bound to L1 and to L2, wherein PB comprises a hydroxyphenyl moiety having a phosphate moiety covalently bound to PB through the oxygen of the hydroxyphenyl moiety, and
L2 is a linker covalently bound to E3LB and PB.

In embodiments, the disclosed compound has the chemical structure:

L1-D,
wherein,
L1 is a peptidomimetic linker covalently bound to D; and
D is a CIDE having the structure:

E3LB-L2-PB,
wherein E3LB is an E3 ligase binding (E3LB) ligand covalently bound to L2, wherein the E3 ligase is von Hippel-Lindau (VHL) tumor suppressor protein, and wherein E3LB comprises a hydroxyproline residue;
PB is a protein binding group covalently bound to L1 and to L2, wherein PB comprises a hydroxyphenyl moiety; and
L2 is a linker covalently bound to E3LB and PB,
wherein a phosphate moiety is covalently bound through the oxygen of a hydroxyphenyl moiety of the PB, or the phosphate moiety is covalently bound through the oxygen of a hydroxyproline residue of the E3LB.

In embodiments, the disclosed conjugate has the chemical structure:

Ab-(L1-D)ⱼ,
wherein,
D is a CIDE having the structure:

E3LB-L2-PB,
wherein,
E3LB is an E3 ligase binding (E3LB) ligand covalently bound to L2, wherein the E3 ligase is von Hippel-Lindau (VHL) tumor suppressor protein, and wherein E3LB comprises a hydroxyproline residue having a phosphate moiety covalently bound through the oxygen of the hydroxyproline residue,
PB is a protein binding group covalently bound to L1 and to L2, and
L2 is a linker covalently bound to E3LB and PB;
Ab is an antibody covalently bound to L1;
L1 is a peptidomimetic linker covalently bound to Ab and D; and
j has a value of from about 1 to about 16.

In embodiments, the disclosed conjugate has the chemical structure:

Ab-(L1-D)ⱼ,
wherein,
D is a CIDE having the structure:

E3LB-L2-PB,
wherein
E3LB is an E3 ligase binding (E3LB) ligand covalently bound to L2, wherein the E3 ligase is von Hippel-Lindau (VHL) tumor suppressor protein,
PB is a protein binding group covalently bound to L1 and to L2, wherein the PB comprises a hydroxyphenyl moiety having a phosphate moiety covalently bound through the oxygen of the hydroxyphenyl moiety, and
L2 is a linker covalently bound to E3LB and PB;
Ab is an antibody covalently bound to L1;
L1 is a peptidomimetic linker covalently bound to Ab and D; and
j has a value of from about 1 to about 16.

In embodiments, the disclosed conjugate has the chemical structure:

Ab-(L1-D)ⱼ,
wherein,
D is a CIDE having the structure:

E3LB-L2-PB,
wherein, E3LB is an E3 ligase binding (E3LB) ligand covalently bound to L2, wherein the E3 ligase is von Hippel-Lindau (VHL) tumor suppressor protein, and wherein E3LB comprises a hydroxyproline residue;
L2 is a linker covalently bound to E3LB and PB, and
PB is a protein binding group covalently bound to L1 and to L2, wherein PB comprises a hydroxyphenyl moiety;
Ab is an antibody covalently bound to L1;
L1 is a peptidomimetic linker covalently bound to Ab and D; and
j has a value of from about 1 to about 16,
wherein a phosphate moiety is covalently bound through the oxygen of a hydroxyphenyl moiety of the PB, or the phosphate moiety is covalently bound through the oxygen of a hydroxyproline residue of the E3LB.

### IV. Formulations

Pharmaceutical formulations of therapeutic Ab-L1-CIDEs as described herein can be prepared for parenteral administration, e.g., bolus, intravenous, intratumor injection with a pharmaceutically acceptable parenteral vehicle and in a unit dosage injectable form. An Ab-CIDE having the desired degree of purity is optionally mixed with one or more pharmaceutically acceptable excipients (Remington's Pharmaceutical Sciences (1980) 16th edition, Osol, A. Ed.), in the form of a lyophilized formulation for reconstitution or an aqueous solution.

An Ab-L1-CIDE can be formulated in accordance with standard pharmaceutical practice as a pharmaceutical composition. According to this aspect, there is provided a pharmaceutical composition comprising an Ab-L1-CIDE in association with one or more pharmaceutically acceptable excipients.

A typical formulation is prepared by mixing an Ab-L1-CIDE with excipients, such as carriers and/or diluents. Suitable carriers, diluents and other excipients are well known to those skilled in the art and include materials such as carbohydrates, waxes, water soluble and/or swellable polymers, hydrophilic or hydrophobic materials, gelatin, oils, solvents, water and the like. The particular carrier, diluent or other excipient used will depend upon the means and purpose for which the Ab-L1-CIDE is being applied. Solvents are generally selected based on solvents recognized by persons skilled in the art as safe (GRAS) to be administered to a mammal.

In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the Ab-L1-CIDE or aid in the manufacturing of the pharmaceutical product. The formulations may be prepared using conventional dissolution and mixing procedures.

Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment.

The Ab-L1-CIDE formulations can be sterile. In particular, formulations to be used for in vivo administration must be sterile. Such sterilization is readily accomplished by filtration through sterile filtration membranes.

The Ab-L1-CIDE ordinarily can be stored as a solid composition, a lyophilized formulation or as an aqueous solution.

The pharmaceutical compositions comprising an Ab-L1-CIDE can be formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the compound to be administered will be governed by such considerations, and is the minimum amount necessary to prevent, ameliorate, or treat the coagulation factor mediated disorder. Such amount is preferably below the amount that is toxic to the host or renders the host significantly more susceptible to bleeding.

The Ab-L1-CIDE can be formulated into pharmaceutical dosage forms to provide an easily controllable dosage of the drug and to enable patient compliance with the prescribed regimen. The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical compositions may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such 1,3-butanediol. The sterile injectable preparation may also be prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of Ab-L1-CIDE that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 g of the active ingredient per milliliter of solution in order that infusion of a suitable volume at a rate of about 30 mL/hr can occur.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The subject matter further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally or by any other desired route.

### V. Indications and Methods of Treatment

It is contemplated that the Ab-L1-CIDEs disclosed herein may be used to treat various diseases or disorders. Also provided herein is an Ab-L1-CIDE or a composition comprising an Ab-L1-CIDE for use in therapy. In some embodiments, provided herein is an Ab-L1-CIDE or a composition comprising an Ab-L1-CIDE for the treatment or prevention of diseases and disorders as disclosed herein. Also provided herein is the use of an Ab-L1-CIDE or a composition comprising an Ab-L1-CIDE in therapy. In some embodiments, provided herein is the use of an Ab-L1-CIDE for the treatment or prevention of diseases and disorders as disclosed herein. Also provided herein is the use of an Ab-L1-CIDE or a composition comprising an Ab-L1-CIDE in the manufacture of a medicament for the treatment or prevention of diseases and disorders as disclosed herein.

Generally, the disease or disorder to be treated is a target protein-dependent disease or disorder, for example, a hyperproliferative disease such as cancer. Examples of cancer to be treated herein include BRM-dependent cancers. In certain embodiments, the cancer is non-small cell lung cancer.

In certain embodiments, the subject matter described herein is directed to a method of reducing the level of a target protein in a subject comprising, administering an Ab-L1-CIDE as described herein or composition comprising an Ab-L1-CIDE as described herein to a subject, wherein the PB portion binds a target protein, wherein ubiquitin ligase effects degradation of a bound target protein, wherein the level of a target protein is reduced.

An Ab-L1-CIDE may be administered by any route appropriate to the condition to be treated. The Ab-L1-CIDE will typically be administered parenterally, i.e. infusion, subcutaneous, intramuscular, intravenous, intradermal, intrathecal and epidural.

An Ab-L1-CIDE can be used either alone or in combination with other agents in a therapy. For instance, an Ab-L1-CIDE may be co-administered with at least one additional therapeutic agent. Such combination therapies noted above encompass combined administration (where two or more therapeutic agents are included in the same or separate formulations), and separate administration, in which case, administration of the Ab-L1-CIDE can occur prior to, simultaneously, and/or following, administration of the additional therapeutic agent and/or adjuvant. An Ab-L1-CIDE can also be used in combination with radiation therapy.

An Ab-L1-CIDE (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

For the prevention or treatment of disease, the appropriate dosage of an Ab-L1-CIDE (when used alone or in combination with one or more other additional therapeutic agents) will depend on the type of disease to be treated, the type of Ab-L1-CIDE, the severity and course of the disease, whether the Ab-L1-CIDE is administered for preventive or therapeutic purposes, previous therapy, the patient's clinical history and response to the Ab-L1-CIDE, and the discretion of the attending physician. The Ab-L1-CIDE is suitably administered to the patient at one time or over a series of treatments. Depending on the type and severity of the disease, about 1 μg/kg to 15 mg/kg (e.g. 0.1 mg/kg-10 mg/kg) of an Ab-L1-CIDE can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of an Ab-L1-CIDE would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses). An initial higher loading dose, followed by one or more lower doses may be administered. However, other dosage regimens may be useful. The progress of this therapy is easily monitored by conventional techniques and assays.

The methods described herein include methods of degrading target proteins. In certain embodiments, the methods comprise administering an Ab-L1-CIDE to a subject, wherein the target protein is degraded. The level of degradation of the protein can be from about 1% to about 5%; or from about 1% to about 10%; or from about 1% to about 15%; or from about 1% to about 20%; from about 1% to about 30%; or from about 1% to about 40%; from about 1% to about 50%; or from about 10% to about 20%; or from about 10% to about 30%; or from about 10% to about 40%; or from about 10% to about 50%; or at least about 1%; or at least about 10%; or at least about 20%; or at least about 30%; or at least about 40%; or at least about 50%; or at least about 60%; or at least about 70%; or at least about 80%; or at least about 90%; or at least about 95%; or at least about 99%.

The methods described herein include methods of reducing proliferation of a neoplastic tissue, such as non-small cell lung cancer. In certain embodiments, the methods comprise administering an Ab-L1-CIDE to a subject, wherein the proliferation of a neoplastic tissue is reduced. The level of reduction can be from about 1% to about 5%; or from about 1% to about 10%; or from about 1% to about 15%; or from about 1% to about 20%; from about 1% to about 30%; or from about 1% to about 40%; from about 1% to about 50%; or from about 10% to about 20%; or from about 10% to about 30%; or from about 10% to about 40%; or from about 10% to about 50%; or at least about 1%; or at least about 10%; or at least about 20%; or at least about 30%; or at least about 40%; or at least about 50%; or at least about 60%; or at least about 70%; or at least about 80%; or at least about 90%; or at least about 95%; or at least about 99%.

### VI. Articles of Manufacture

In another aspect, described herein are articles of manufacture, for example, a “kit,” containing materials useful for the treatment of the diseases and disorders described above is provided. The kit comprises a container comprising an Ab-L1-CIDE. The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products.

Suitable containers include, for example, bottles, vials, syringes, blister pack, etc. A “vial” is a container suitable for holding a liquid or lyophilized preparation. In one embodiment, the vial is a single-use vial, e.g. a 20-cc single-use vial with a stopper. The container may be formed from a variety of materials such as glass or plastic. The container may hold an Ab-L1-CIDE or a formulation thereof which is effective for treating the condition and may have a sterile access port (for example, the container may be an intravenous solution bag or a vial having a stopper pierceable by a hypodermic injection needle).

At least one active agent in the composition is an Ab-L1-CIDE. The label or package insert indicates that the composition is used for treating the condition of choice, such as cancer. In addition, the label or package insert may indicate that the patient to be treated is one having a disorder such as a hyperproliferative disorder, neurodegeneration, cardiac hypertrophy, pain, migraine or a neurotraumatic disease or event. In one embodiment, the label or package inserts indicates that the composition comprising an Ab-L1-CIDE can be used to treat a disorder resulting from abnormal cell growth. The label or package insert may also indicate that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the Ab-L1-CIDE and, if present, the second pharmaceutical formulation. For example, if the kit comprises a first composition comprising an Ab-L1-CIDE, and a second pharmaceutical formulation, the kit may further comprise directions for the simultaneous, sequential or separate administration of the first and second pharmaceutical compositions to a patient in need thereof.

In another embodiment, the kits are suitable for the delivery of solid oral forms of an Ab-L1-CIDE, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to one embodiment, a kit may comprise (a) a first container with an Ab-L1-CIDE contained therein; and optionally (b) a second container with a second pharmaceutical formulation contained therein, wherein the second pharmaceutical formulation comprises a second compound with anti-hyperproliferative activity. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In certain other embodiments wherein the kit comprises an Ab-L1-CIDE and a second therapeutic agent, the kit may comprise a container for containing the separate compositions such as a divided bottle or a divided foil packet; however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

### VII. Methods of Making Conjugates

### Synthesis Routes

The subject matter described herein is also directed to methods of preparing a CIDE, a L1-CIDE, and an Ab-L1-CIDE from a L1-CIDE. Generally, the method comprises contacting an antibody, or variants, mutations, splice variants, indels and fusions thereof, with a L1-CIDE under conditions where the antibody is covalently bound to any available point of attachment on a L1-CIDE, wherein an Ab-L1-CIDE is prepared. The subject matter described herein is also directed to methods of preparing an Ab-L1-CIDE from an Ab-L1 portion, i.e., an antibody, or variants, mutations, splice variants, indels and fusions thereof, covalently attached to a L1, the methods comprising contacting a CIDE with an Ab-L1 under conditions where the CIDE is covalently bound to any available point of attachment on the Ab-L1, wherein an Ab-L1-CIDE is prepared. The methods can further comprise routine isolation and purification of the Ab-L1-CIDEs.

CIDEs, L1-CIDEs and Ab-L1-CIDEs and other compounds described herein can be synthesized by synthetic routes that include processes analogous to those well-known in the chemical arts, particularly in light of the description contained herein, and those for other heterocycles described in: Comprehensive Heterocyclic Chemistry II, Editors Katritzky and Rees, Elsevier, 1997, e.g. Volume 3; Liebigs Annalen der Chemie, (9):1910-16, (1985); Helvetica Chimica Acta, 41:1052-60, (1958); Arzneimittel-Forschung, 40(12):1328-31, (1990). Starting materials are generally available from commercial sources such as Aldrich Chemicals (Milwaukee, WI) or are readily prepared using methods well known to those skilled in the art (e.g., prepared by methods generally described in Louis F. Fieser and Mary Fieser, Reagents for Organic Synthesis, v. 1-23, Wiley, N.Y. (1967-2006 ed.), or Beilsteins Handbuch der organischen Chemie, 4, Aufl. ed. Springer-Verlag, Berlin, including supplements (also available via the Beilstein online database).

Synthetic chemistry transformations and protecting group methodologies (protection and deprotection) useful in synthesizing the CIDEs, L1-CIDEs and Ab-L1-CIDEs and other compounds as described herein and necessary reagents and intermediates are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); and L. Paquette, ed., Encyclopedia of Reagents for Organic Synthesis, John Wiley and Sons (1995) and subsequent editions thereof. In preparing CIDEs, L1-CIDEs and Ab-CIDEs and other compounds, protection of remote functionality (e.g., primary or secondary amine) of intermediates may be necessary. The need for such protection will vary depending on the nature of the remote functionality and the conditions of the preparation methods. Suitable amino-protecting groups include acetyl, trifluoroacetyl, t-butoxycarbonyl (BOC), benzyloxycarbonyl (CBz or CBZ) and 9-fluorenylmethyleneoxycarbonyl (Fmoc). The need for such protection is readily determined by one skilled in the art. For a general description of protecting groups and their use, see T. W. Greene, Protective Groups in Organic Synthesis, John Wiley & Sons, New York, 1991.

The General Procedures and Examples provide exemplary methods for preparing CIDEs, L1-CIDEs and Ab-L1-CIDEs and other compounds described herein. Those skilled in the art will appreciate that other synthetic routes may be used to synthesize the Ab-L1-CIDEs and compounds. Although specific starting materials and reagents are depicted and discussed in the Schemes, General Procedures, and Examples, other starting materials and reagents can be easily substituted to provide a variety of derivatives and/or reaction conditions. In addition, many of the exemplary compounds prepared by the described methods can be further modified in light of this disclosure using conventional chemistry well known to those skilled in the art.

Generally, an Ab-L1-CIDE can be prepared by connecting a CIDE with a L1 linker reagent according to the procedures of WO 2013/055987; WO 2015/023355; WO 2010/009124; WO 2015/095227, to prepare a L1-CIDE, and conjugating the L1-CIDE with any of the antibodies or variants, mutations, splice variants, indels and fusions thereof, including cysteine engineered antibodies, described herein. Alternatively, an Ab-CIDE can be prepared by first connecting an antibody or variant, mutation, splice variant, indel and fusion thereof, including a cysteine engineered antibody, described herein with a L1 linker reagent, and conjugating it with any CIDE.

The following synthetic routes describe exemplary methods of preparing CIDEs, L1-CIDEs and Ab-L1-CIDEs and other compounds and components thereof. Other synthetic routes for preparing CIDEs, L1-CIDEs and Ab-L1-CIDEs and other compounds and components thereof are disclosed elsewhere herein.

### 1. Cysteine Engineered Antibodies

Cysteine engineered antibodies (THIOMABs™) can be expressed and purified recombinantly using standard methods, and can generally be prepared for conjugation by reduction and reoxidation as follows.

Full length, cysteine engineered monoclonal antibodies (THIOMAB™ antibodies) expressed recombinantly bear cysteine adducts (cystines) or are glutathionylated on the engineered cysteines due to cell culture conditions. As is, THIOMAB™ antibodies purified from standard mammalian cell lines cannot be conjugated to Cys-reactive linker L1-CIDE intermediates. Cysteine engineered antibodies may be made reactive for conjugation with L1-PCIDE intermediates described herein, by treatment with a reducing agent such as DTT (Cleland's reagent, dithiothreitol) or TCEP (tris(2-carboxyethyl)phosphine hydrochloride; Getz et al (1999) Anal. Biochem. Vol 273:73-80; Soltec Ventures, Beverly, MA) followed by re-formation of the inter-chain disulfide bonds (re-oxidation) with a mild oxidant such as dehydroascorbic acid. Full length, cysteine engineered monoclonal antibodies (THIOMAB™ antibodies) expressed in CHO cells (Gomez et al (2010) Biotechnology and Bioeng. 105(4):748-760; Gomez et al (2010) Biotechnol. Prog. 26:1438-1445) were reduced, for example, with about a 50 fold excess of DTT overnight in 50 mM Tris, pH 8.0 with 2 mM EDTA at room temperature, which removes Cys and glutathione adducts as well as reduces interchain disulfide bonds in the antibody. Removal of the adducts was monitored by reverse-phase LCMS using a PLRP-S column.

After the removal of Cys and glutathione adducts, THIOMAB™ antibodies can be purified by methods known commonly in the art, including cation exchange chromatography which is elaborated here. Reduced THIOMABs™ can be diluted and acidified by adding to at least four volumes of 10 mM succinate, pH 5 and/or titration with 10% acetic acid until the pH is approximately five. The pH-lowered and diluted THIOMAB™ antibody can be subsequently loaded onto a HiTrap S cation exchange column, washed with several column volumes of 10 mM sodium acetate, pH 5 and eluted with 50 mM Tris, pH 8.0, 150 mM sodium chloride. Disulfide bonds can be reestablished between cysteine residues present in the parent Mab by carrying out reoxidation. The eluted reduced THIOMAB™ antibody described above can be treated with 15× dehydroascorbic acid (DHAA) for about 3 hours or, alternatively, with 200 nM to 2 mM aqueous copper sulfate (CuSO₄) at room temperature overnight. Other oxidants, i.e. oxidizing agents, and oxidizing conditions, which are known in the art may be used. Ambient air oxidation may also be effective. This mild, partial reoxidation step forms intrachain disulfides efficiently with high fidelity. Reoxidation can be monitored by reverse-phase LCMS using a PLRP-S column. The reoxidized THIOMAB™ antibody can then be diluted with succinate buffer as described above to reach pH approximately 5, followed by purification on an S column as described above with the exception that elution was performed with a gradient of 10 mM succinate, pH 5, 300 mM sodium chloride (buffer B) and 10 mM succinate, pH 5 (buffer A). To the eluted THIOMAB™ antibody, EDTA can be added to a final concentration of 2 mM. The THIOMAB™ can be concentrated, if necessary, to reach a final concentration of more than 5 mg/mL. The resulting THIOMAB™ antibody, ready for conjugation, can be stored at −20° C. or −80° C. Liquid chromatography/Mass Spectrometric Analysis can be performed on a 6200 series TOF or QTOF Agilent LC/MS. Samples are chromatographed on a PRLP-S®, 1000 A, microbore column (50 mm×2.1 mm, Polymer Laboratories, Shropshire, UK) heated to 80° C. A linear gradient from 30-40% B (solvent A: 0.05% TFA in water, solvent B: 0.04% TFA in acetonitrile) can be used and the eluent is directly ionized using the electrospray source. Data were collected and deconvoluted by the MassHunter software (Agilent). Prior to LC/MS analysis, antibodies (50 micrograms) can be treated with PNGase F (2 units/ml; PROzyme, San Leandro, CA) for 2 hours at 37° C. to remove N-linked carbohydrates.

Alternatively, antibodies can be partially digested with LysC (0.25 μg per 50 μg (microgram) antibody) for 15 minutes at 37° C. to give a Fab and Fc fragment for analysis by LCMS.

### 2. Conjugation of Linker L1-CIDE Group to Antibodies

In one method of conjugating Linker L1-PCIDE compounds to antibodies, after the reduction and reoxidation procedures above, the cysteine-engineered antibody (THIOMAB™ antibody), in 10 mM succinate, pH 5, 150 mM NaCl, 2 mM EDTA, is pH-adjusted to pH 7.5-8.5 with 1M Tris. An excess, from about 3 molar to 20 equivalents of a linker-PCIDE intermediate with a thiol-reactive group (e.g., maleimide or 4-nitropyridy disulfide, or methanethiosulfonyl (MTS) disulfide), is dissolved in DMF or DMA, with or without or propylene glycol before addition to the reduced, reoxidized, and pH-adjusted antibody. The reaction is incubated at room temperature or 37 C and monitored until completion (1 to about 24 hours), as determined by LC-MS analysis of the reaction mixture. When the reaction is complete, the conjugate is purified by one or any combination of several methods, the goal being to remove remaining unreacted L1-PCIDE intermediate and aggregated protein (if present at significant levels). For example, the conjugate may be diluted with 10 mM histidine-acetate, pH 5.5 until final pH is approximately 5.5 and purified by S cation exchange chromatography using either HiTrap S columns connected to an Akta purification system (GE Healthcare) or S maxi spin columns (Pierce). Alternatively, the conjugate may be purified by gel filtration chromatography using an S200 column connected to an Akta purification system or Zeba spin columns. Alternatively, dialysis may be used to remove unreacted or excess linker drug. The THIOMAB™ antibody PCIDE conjugates can be formulated into 20 mM His/acetate, pH 5, with 240 mM sucrose using either gel filtration or dialysis. The purified conjugate can be concentrated by centrifugal ultrafiltration and/or filtered through a 0.2-μm filter under sterile conditions and frozen for storage. The Ab-L1-PCIDEs were characterized by BCA assay to determine protein concentration, analytical SEC (size-exclusion chromatography) for aggregation analysis and LC-MS after treatment with Lysine C endopeptidase (LysC) or reduction using standard procedures to calculate DAR.

Size exclusion chromatography is performed on conjugates using a Shodex KW802.5 column in 0.2M potassium phosphate pH 6.2 with 0.25 mM potassium chloride and 15% IPA at a flow rate of 0.75 ml/min. Aggregation state of the conjugate was determined by integration of eluted peak area absorbance at 280 nm.

LC-MS analysis may be performed on Ab-L1-PCIDE using an Agilent QTOF 6520 ESI instrument. As an example, the Ab-L1-PCIDE is treated with 1:500 w/w Endoproteinase Lys C (Promega) in Tris, pH 7.5, for 30 min at 37° C. The resulting cleavage fragments are loaded onto a 1000 (Angstrom), 8 μm (micron) PLRP-S (highly cross-linked polystyrene) column heated to 80° C. and eluted with a gradient of 30% B to 40% B in 5 minutes. Mobile phase A was H₂O with 0.05% TFA and mobile phase B was acetonitrile with 0.04% TFA. The flow rate was 0.5 ml/min. Protein elution was monitored by UV absorbance detection at 280 nm prior to electrospray ionization and MS analysis. Chromatographic resolution of the unconjugated Fc fragment, residual unconjugated Fab and drugged Fab was usually achieved. The obtained m/z spectra were deconvoluted using Mass Hunter™ software (Agilent Technologies) to calculate the mass of the antibody fragments. Peaks in the deconvoluted LCMS spectra can be assigned and quantitated. PCIDE-to-antibody ratios (DAR) are calculated by calculating the ratio of intensities of the peak or peaks corresponding to PCIDE-conjugated antibody relative to all peaks observed.

### 3. General Synthetic Coupling of L2 to E3LB to Prepare a E3LB-L2 Intermediate

In certain embodiments, L2 is first contacted with a first suitable solvent, a first base and a first coupling reagent to prepare a first solution. In certain embodiments, the contacting of L2 with a first suitable solvent, a first base, and a first coupling reagent proceeds for about 15 minutes at room temperature (about 25° C.). The E3LB is then contacted with said first solution.

In certain embodiments, the contacting of E3LB with the first solution proceeds for about one hour at room temperature (about 25° C.). The solution is then concentrated and optionally purified.

In certain embodiments, the molar ratio of L2 to first base to first coupling reagent is about 1:4:1.19. In certain embodiments, the molar ratio of L2 to first base to first coupling reagent is about 1:2:0.5, about 1:3:1, about 1:4:2, about 1:5:3, or about 1:6:4.

In certain embodiments, the molar ratio of L2 to E3LB is about 1:1. In certain embodiments, the molar ratio of L2 to E3LB is about 1:0.5, about 1:0.75, about 1:2, or about 0.5:1.

### 4. General Synthetic Method for Coupling E3LB-L2 Intermediate to PB

In certain embodiments, the E3LB-L2 intermediate is coupled to a PB to prepare a CIDE. In certain embodiments, the PB is first contacted with a second suitable solvent, a second base, and second coupling reagent. In certain embodiments, the contacting proceeds for about 10 minutes at room temperature (about 25° C.). The solution is then contacted with the E3LB-L2 intermediate. In certain embodiments, the contacting of the second solution with the E3LB-L2 intermediate proceeds for about 1 hour at room temperature (about 25° C.). The solution is then concentrated and optionally purified to prepare a CIDE.

In certain embodiments, the molar ratio of PB to second base to second coupling reagent is about 1:4:1.2. In certain embodiments, the molar ratio of PB to second base to second coupling reagent is about 1:3:0.75, about 1:5:1, about 1:3:2, or about 1:5:3.

In certain embodiments, the molar ratio of PB to E3LB-L2 intermediate is about 1:1. In certain embodiments, the molar ratio of PB to E3LB-L2 intermediate is about 1:0.5, about 1:0.75, about 1:2, or about 0.5:1.

### 5. General Synthetic Method for Coupling CIDE to L1 to Prepare L1-CIDE

In certain embodiments, the CIDE is contacted with L1 and a third base in a third suitable solvent to prepare a solution. In certain embodiments, the contacting proceeds for about 2 hours at about (about 25° C.). The solution can then be optionally purified to prepare L1-CIDE.

In certain embodiments, the molar ratio of CIDE to L1 is about 1:4. In certain embodiments, the molar ratio of CIDE to L1 is about 1:1, 1:2, 1:3, 1:5, 1:6, 1:7, or about 1:8.

### 6. General Synthetic Method for Coupling L1-CIDE to Antibody

In certain embodiments, the L1-CIDE is contacted with a thiol and a fourth suitable solvent to form a fourth solution. This solution is then contacted with an antibody to prepare the conjugate. In certain embodiments, the

In certain embodiments, the thiol is maleimide or 4-nitropyridy disulfide. In certain embodiments, the suitable solvent is selected from the group consisting of dimethylformamide, dimethylacetamide, and propylene glycol.

In certain embodiments, the molar ratio of L1-CIDE to thiol-reactive group is about 3:1 to about 20:1.

In certain embodiments, contacting the solution comprising the L1-CIDE, the thiol-reactive group and the suitable solvent with the antibody proceeds for about 1 to about 24 hours. In certain embodiments, contacting the solution comprising the L1-CIDE, the thiol-reactive group and the suitable solvent with the antibody proceeds at about room temperature (about 25° C.) to about 37° C.

In certain embodiments of the general methods above, the suitable solvent is a polar aprotic solvent, selected from the group consisting of dimethylformamide, tetrahydrofuran, ethyl acetate, acetone, acetonitrile, dimethyl sulfoxide, and propylene carbonate.

In certain embodiments of the general methods above, the base is selected from the group consisting of N,N-Diisopropylethylamine (DIEA), triethylamine, and 2,2,2,6,6-tetramethylpiperidine. In certain embodiments, the coupling reagent is selected from the group consisting of 1-[Bis(dimethylamino)methylene]-1H-1,2,3-triazolo[4,5-b]pyridinium 3-oxide hexafluorophosphate (HATU), (Benzotriazol-1-yloxy)tris(dimethylamino)phosphionium hexafluorophosphate (BOP), (7-Azabenzotriazol-1-yloxy)tripyrrolidinophosphonium hexafluorophosphate (PyAOP), O-(Benzotriazol-1-yl)-N,N,N′,N′-tetramethyluronium hexafluorophosphate (HBTU), O-(Benzotriazol-1-yl)-N,N,N′,N′-tetramethyluronium tetrafluoroborate (TBTU), O-(6-Chlorobenzotriazol-1-yl)-N,N,N′,N′-tetramethyluronium hexafluorophosphate (HCTU), O-(N-Suc-cinimidyl)-1,1,3,3-tetramethyl-uronium tetrafluoroborate (TSTU), O-(5-Norbornene-2,3-dicarboximido)-N,N,N′,N′-tetramethyluronium tetrafluoroborate (TNTU), O-(1,2-Dihydro-2-oxo-1-pyridyl-N,N,N′,N′-tetramethyluronium tetrafluoroborate (TPTU), and Carbonyldiimidazole (CDI).

In certain embodiments of the general methods above, contacting proceeds for about 30 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 30 minutes, 60 minutes, 90 minutes, 120 minutes, 180 minutes, 4 hours, 5 hours, 6 hours, 7 hours, 8 hours, 9 hours, 10 hours, 20 hours, 40 hours, 60 hours, or 72 hours.

In certain embodiments of the general methods above, contacting proceeds at about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 60° C., 70° C., 80° C., 90° C., or 100° C.

The following examples are offered by way of illustration and not by way of limitation.

### EXAMPLES

### Example 1

### Synthesis of L1Hy-PE-CIDE-1

### L1Hy-PE-CIDE-1: (3R,5S)-1-((2R)-2-(3-(2-(4-((1r,3R)-3-((4-(3-(3-Amino-6-(2-((4-((S)-6-(dimethylamino)-2-(1-((2-((2-(2,5-dioxo-2,5-dihydro-1H-pyrrol-1-yl)ethyl)(methyl)amino)ethyl)carbamoyl)cyclobutane-1-carboxamido)hexanamido)benzyl)oxy)phenyl)pyridazin-4-yl)-3,8-diazabicyclo[3.2.1]octan-8-yl)pyridin-2-yl)oxy)cyclobutoxy)piperidin-1-yl)ethoxy)isoxazol-5-yl)-3-methylbutanoyl)-5-(((S)-1-(4-cyanophenyl)ethyl)carbamoyl)pyrrolidin-3-yl dihydrogen phosphate (2,2,2-trifluoroacetic acid salt)

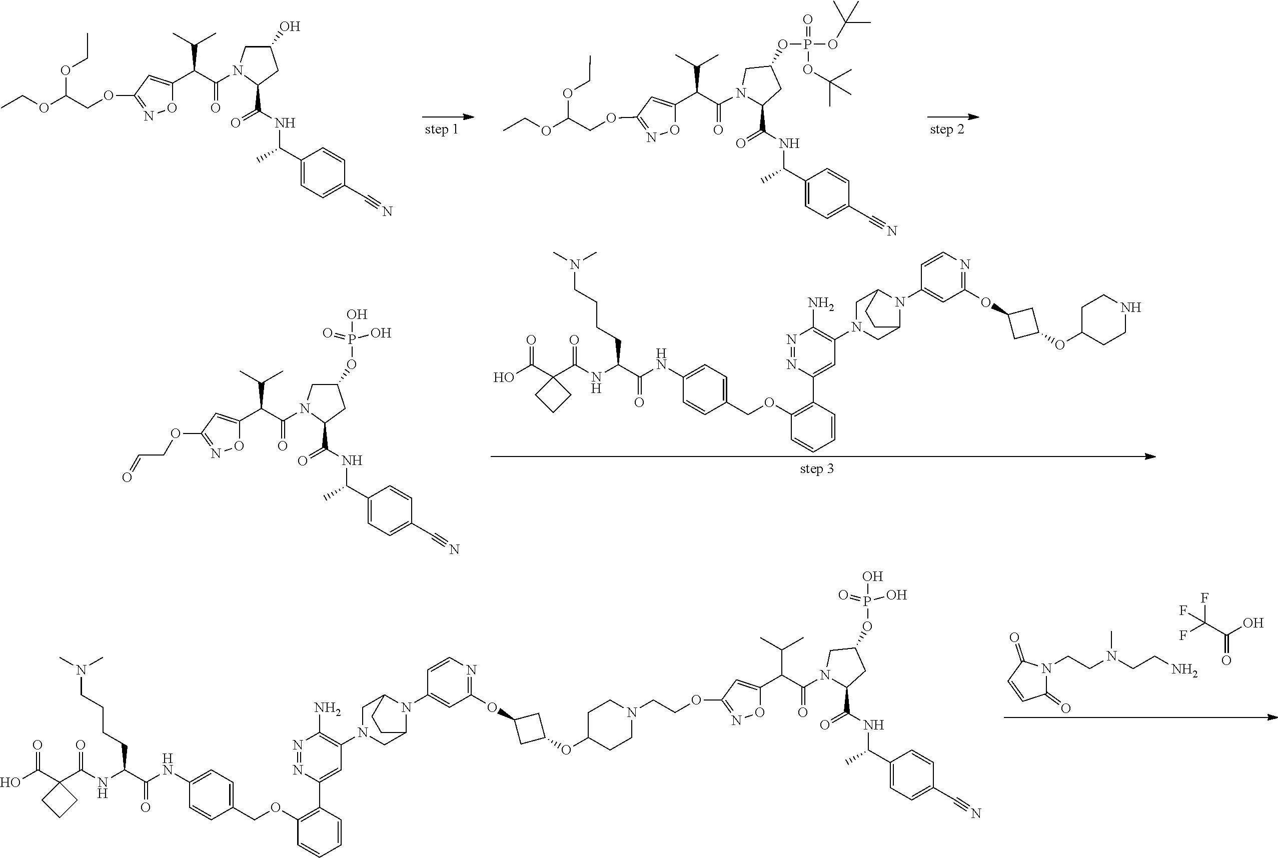

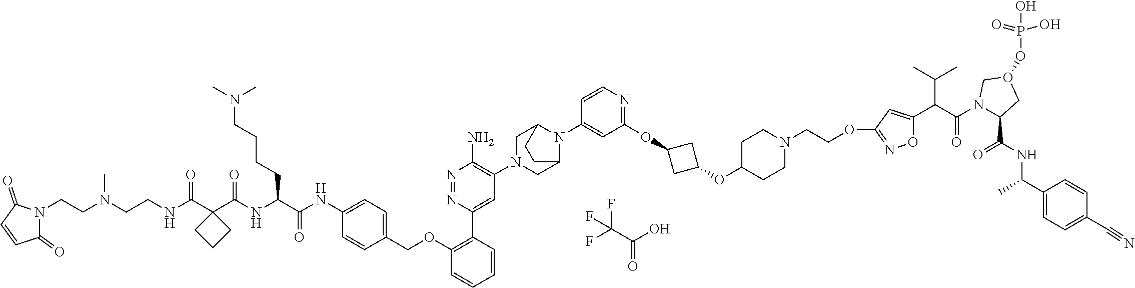

### Step 1: di-tert-butyl ((3R,5)-5-(((S)-1-(4-cyanophenyl)ethyl)carbamoyl)-1-((R)-2-(3-(2,2-diethoxyethoxy)isoxazol-5-yl)-3-methylbutanoyl)pyrrolidin-3-yl) phosphate

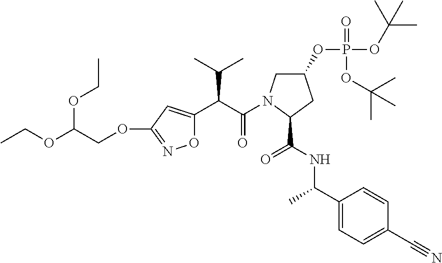 [SMILES: CCOC(COc1cc([C@H](C(=O)N2C[C@H](OP(=O)(OC(C)(C)C)OC(C)(C)C)C[C@H]2C(=O)N[C@@H](C)c2ccc(C#N)cc2)C(C)C)on1)OCC]

Under nitrogen, to a solution of (2S,4R)—N—((S)-1-(4-cyanophenyl)ethyl)-1-((R)-2-(3-(2,2-diethoxyethoxy)isoxazol-5-yl)-3-methylbutanoyl)-4-hydroxypyrrolidine-2-carboxamide (500 mg, 0.921 mmol) and 1H-tetrazole (6.1 mL, 0.45 M in ACN) in THF (15 mL) was added di-tert-butyl diisopropylphosphoramidite (511 mg, 1.84 mmol) at 0° C. The resulting solution was stirred at room temperature overnight. Then t-BuOOH (5M in decane) (0.74 mL, 3.69 mmol) was added at −30° C. and stirred at room temperature for 5 h. The reaction was quenched with NaHSO₃. The resulting solution was extracted with EtOAc and the organic layers were combined. The organic layer was dried over anhydrous sodium sulfate and concentrated under vacuum. The residue was purified by flash chromatography on silica gel (gradient: 0%-87% ethyl acetate/petroleum ether) to yield 420 mg (62% yield) of the title compound as a colorless oil. LC-MS: (ESI, m/z): [M+H]⁺=735.

### Step 2: (3R,5S)-5-(((S)-1-(4-Cyanophenyl)ethyl)carbamoyl)-1-((R)-3-methyl-2-(3-(2-oxoethoxy)isoxazol-5-yl)butanoyl)pyrrolidin-3-yl dihydrogen phosphate

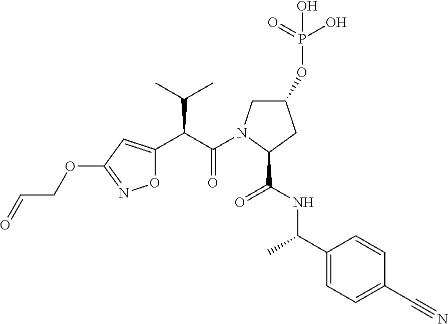 [SMILES: CC(C)[C@@H](C(=O)N1C[C@H](OP(=O)(O)O)C[C@H]1C(=O)N[C@@H](C)c1ccc(C#N)cc1)c1cc(OCC=O)no1]

Under nitrogen, a solution of di-tert-butyl ((3R,5S)-5-(((S)-1-(4-cyanophenyl)ethyl)carbamoyl)-1-((R)-2-(3-(2,2-diethoxyethoxy)isoxazol-5-yl)-3-methylbutanoyl)pyrrolidin-3-yl) phosphate (70.0 mg, 0.100 mmol) in HCOOH (0.5 mL) and water (0.5 mL) was stirred at 60° C. for 1 h. The solution was concentrated under vacuum to afford 50 mg (crude) of the title compound as a solid. LC-MS: (ESI, m/z): [M+H]=549.

### Step 3: 1-(((2S)-1-((4-((2-(6-Amino-5-(8-(2-(3-((1-(2-((5-((R)-1-((2S,4R)-2-(((S)-1-(4-cyanophenyl)ethyl)carbamoyl)-4-(phosphonooxy)pyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid

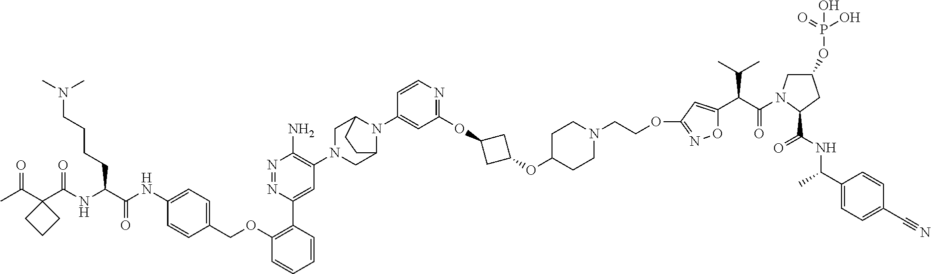 [SMILES: CC(=O)C1(C(=O)N[C@@H](CCCCN(C)C)C(=O)Nc2ccc(COc3ccccc3-c3cc(N4CC5CCC(C4)N5c4ccnc(O[C@H]5C[C@H](OC6CCN(CCOc7cc([C@H](C(=O)N8C[C@H](OP(=O)(O)O)C[C@H]8C(=O)N[C@@H](C)c8ccc(C#N)cc8)C(C)C)on7)CC6)C5)c4)c(N)nn3)cc2)CCC1]

Under nitrogen, a solution of (3R,5S)-5-(((S)-1-(4-cyanophenyl)ethyl)carbamoyl)-1-((R)-3-methyl-2-(3-(2-oxoethoxy)isoxazol-5-yl)butanoyl)pyrrolidin-3-yl dihydrogen phosphate (62.8 mg, 0.115 mmol), 1-(((2S)-1-((4-((2-(6-amino-5-(8-(2-((1r,3r)-3-(piperidin-4-yloxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (56.0 mg, 0.0602 mmol), CH₃COONa (14.9 mg, 0.182 mmol) in methanol (1.5 mL) and dichloromethane (0.5 ml) was stirred at room temperature for 1 hour. Then NaBH₃CN (5.6 mg, 0.0891 mmol) was added and stirred at room temperature for 3 hours. The reaction was quenched by water. The reaction mixture was concentrated under vacuum. The residue was purified by pre-packed C18 column (solvent gradient: 0-100% ACN in water (0.05% NH₄HCO₃)) to yield 60 mg (65% yield) of the title compound as a white solid. LC-MS: (ESI, m z): [M+H]⁺=1464.

### Step 4: (3R,5S)-1-((2R)-2-(3-(2-(4-((1r,3R)-3-((4-(3-(3-Amino-6-(2-((4-((S)-6-(dimethylamino)-2-(1-((2-((2-(2,5-dioxo-2,5-dihydro-1H-pyrrol-1-yl)ethyl)(methyl)amino)ethyl)carbamoyl)cyclobutane-1-carboxamido)hexanamido)benzyl)oxy)phenyl)pyridazin-4-yl)-3,8-diazabicyclo[3.2.1]octan-8-yl)pyridin-2-yl)oxy)cyclobutoxy)piperidin-1-yl)ethoxy)isoxazol-5-yl)-3-methylbutanoyl)-5-(((S)-1-(4-cyanophenyl)ethyl)carbamoyl)pyrrolidin-3-yl dihydrogen phosphate (2,2,2-trifluoroacetic acid salt)

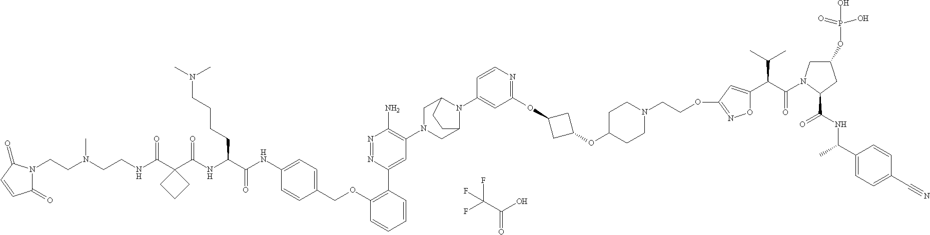 [SMILES: CC(C)[C@@H](C(=O)N1C[C@H](OP(=O)(O)O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCc7ccc(NC(=O)[C@H](CCCCN(C)C)NC(=O)C8(C(=O)NCCN(C)CCN9C(=O)C=CC9=O)CCC8)cc7)nnc5N)C6)ccn4)C3)CC2)no1 ; O=C(O)C(F)(F)F]

To a solution of 1-(((2S)-1-((4-((2-(6-amino-5-(8-(2-(3-((1-(2-((5-((R)-1-((2S,4R)-2-(((S)-1-(4-cyanophenyl)ethyl)carbamoyl)-4-(phosphonooxy)pyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (60.0 mg, 0.0410 mmol), 1-(2-((2-aminoethyl)(methyl)amino)ethyl)-1H-pyrrole-2,5-dione (2,2,2-trifluoroacetic acid salt) (10.5 mg, crude) and DIPEA (54.2 mg, 0.420 mmol) in DMF (1 mL) was added HATU (18.7 mg, 0.0492 mmol) at room temperature. The reaction was stirred at room temperature for 10 minutes. The crude was purified by Prep-HPLC (Column: Xselect CSH C18 OBD Column 30*150 mm 5 μm, n; Mobile Phase A: Water (0.05% TFA), Mobile Phase B: ACN; Flow rate: 60 mL/min; Gradient: 14% B to 23% B in 12 min, 23% B; Wave Length: 254/220 nm; R_{T1}(min): 11.75 min) to yield 20.3 mg (30% yield) of L1Hy-PE-CIDE-1 as a white solid. LC-MS: (ESI, m/z): [M+H]=1643. ¹H NMR (300 MHz, DMSO-d₆) δ 10.24 (s, 1H), 9.67 (s, 1H), 8.56 (d, J=7.3 Hz, 1H), 8.12 (s, 1H), 7.88-7.73 (m, 4H), 7.68-7.49 (m, 4H), 7.49-7.25 (m, 6H), 7.20-6.80 (m, 4H), 6.61 (s, 1H), 6.21-5.95 (m, 2H), 5.30-5.00 (m, 3H), 4.90 (t, J=6.9 Hz, 1H), 4.76 (s, 1H), 4.52-4.27 (m, 7H), 3.76-3.67 (m, 6H), 3.55-3.40 (m, 9H), 3.30-3.20 (m, 5H), 3.15-2.95 (m, 5H), 2.81 (s, 3H), 2.73 (s, 6H), 2.45-2.30 (m, 9H), 2.01-1.54 (m, 15H), 1.49-1.20 (m, 5H), 0.94 (d, J=6.4 Hz, 3H), 0.79 (d, J=7.0 Hz, 3H).

### Example 2

### Synthesis of L1-PE-CIDE-2

### L1-PE-CIDE-2: (3R,5S)-1-((2R)-2-(3-(2-((3R)-4-(2-((4-(3-(3-Amino-6-(2-((4-((S)-2-(1-((5-(2,5-dioxo-2,5-dihydro-1H-pyrrol-1-yl)pentyl)carbamoyl)cyclobutane-1-carboxamido)-5-ureidopentanamido)benzyl)oxy)phenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoyl)-5—(((S)-1-(4-(4-methylthiazol-5-yl)phenyl)ethyl)carbamoyl)pyrrolidin-3-yl dihydrogen phosphate (2,2,2-trifluoroacetic acid salt)

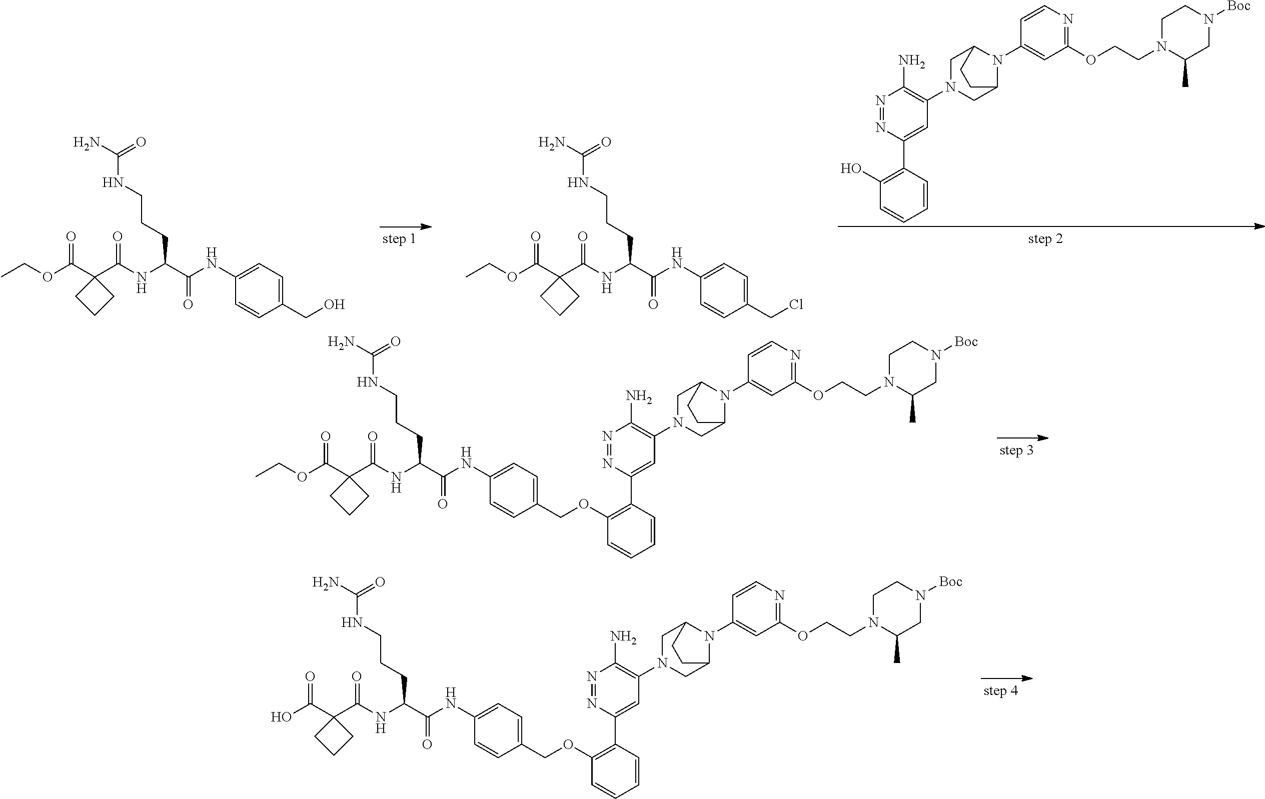

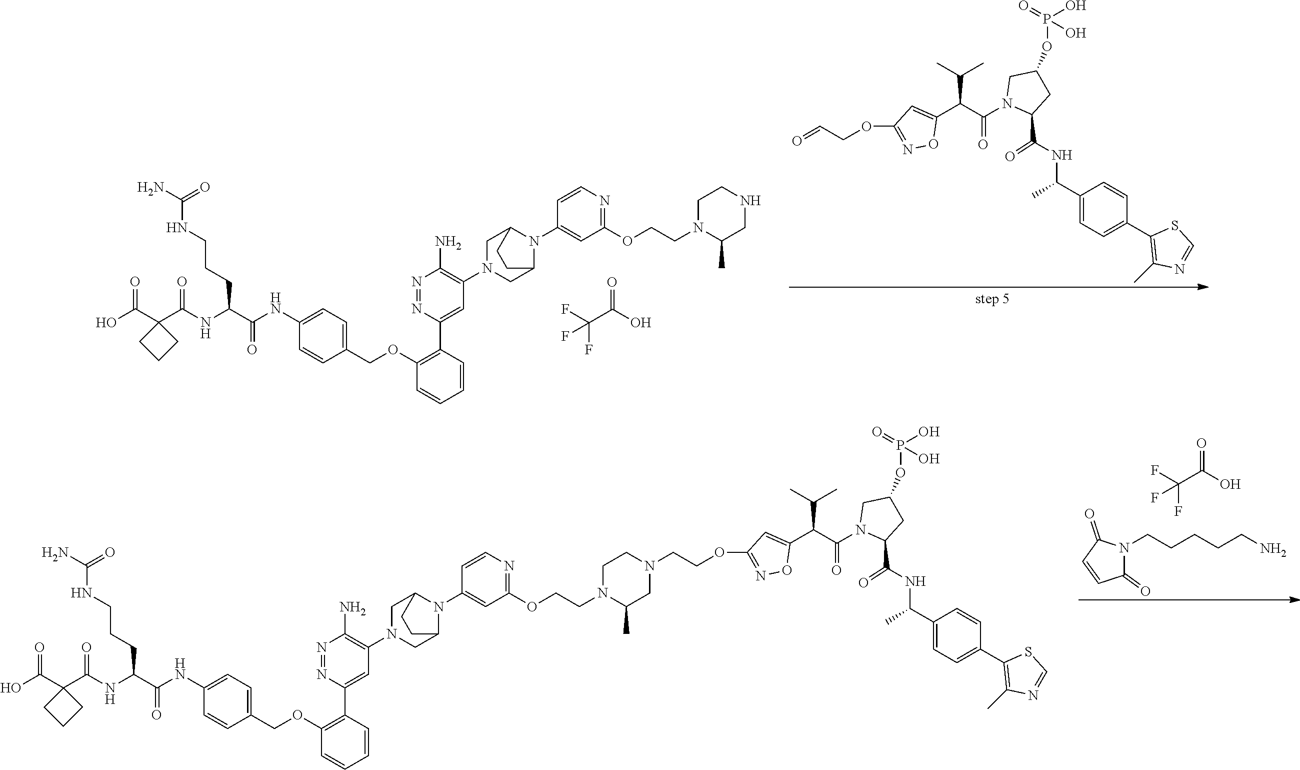

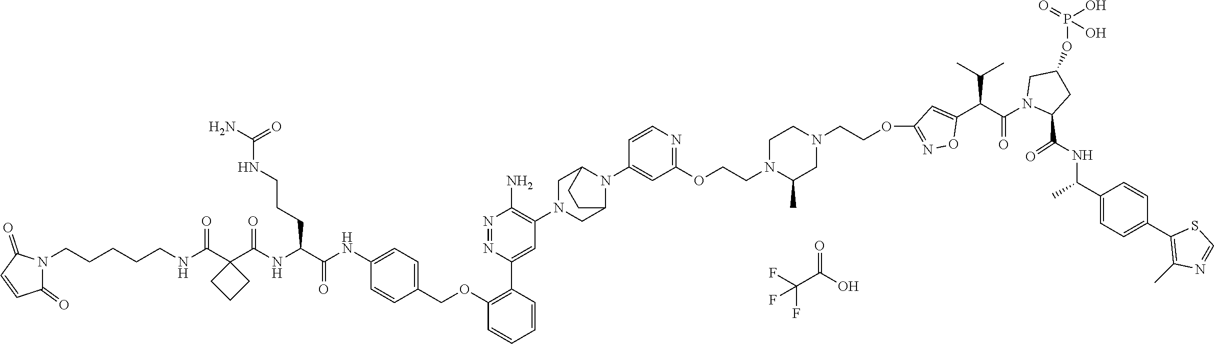 [SMILES: Cc1ncsc1-c1ccc([C@H](C)NC(=O)[C@@H]2C[C@@H](OP(=O)(O)O)CN2C(=O)[C@@H](c2cc(OCCN3CCN(CCOc4cc(N5C6CCC5CN(c5cc(-c7ccccc7OCc7ccc(NC(=O)[C@H](CCCNC(N)=O)NC(=O)C8(C(=O)NCCCCCN9C(=O)C=CC9=O)CCC8)cc7)nnc5N)C6)ccn4)[C@H](C)C3)no2)C(C)C)cc1 ; O=C(O)C(F)(F)F]

Step 1: Ethyl (S)-1-((1-((4-(chloromethyl)phenyl)amino)-1-oxo-5-ureidopentan-2-yl)carbamoyl)cyclobutane-1-carboxylate

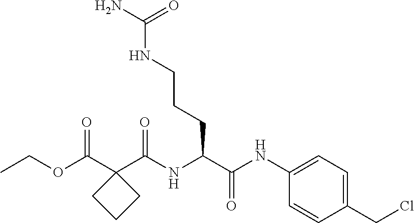 [SMILES: CCOC(=O)C1(C(=O)N[C@@H](CCCNC(N)=O)C(=O)Nc2ccc(CCl)cc2)CCC1]

To a solution of ethyl (S)-1-((1-((4-(hydroxymethyl)phenyl)amino)-1-oxo-5-ureidopentan-2-yl)carbamoyl)cyclobutane-1-carboxylate (1.00 g, 2.30 mmol) in THF (10 mL) was added SOCl₂ (0.830 g, 6.97 mmol) at 0° C. The reaction was stirred at room temperature for 1 h. MeOH was added at 0° C. to quench the reaction. Solvent was evaporated and the crude was purified by flash chromatography on silica gel (solvent gradient: 0-10% MeOH in DCM) to yield 910 mg of the title compound as a white solid. LCMS (ESI) [M+H]=453.

### Step 2: tert-Butyl (3R)-4-(2-((4-(3-(3-amino-6-(2-((4-((S)-2-(1-(ethoxycarbonyl)cyclobutane-1-carboxamido)-5-ureidopentanamido)benzyl)oxy)phenyl)pyridazin-4-yl)-3,8-diazabicyclo[3.2.1]octan-8-yl)pyridin-2-yl)oxy)ethyl)-3-methylpiperazine-1-carboxylate

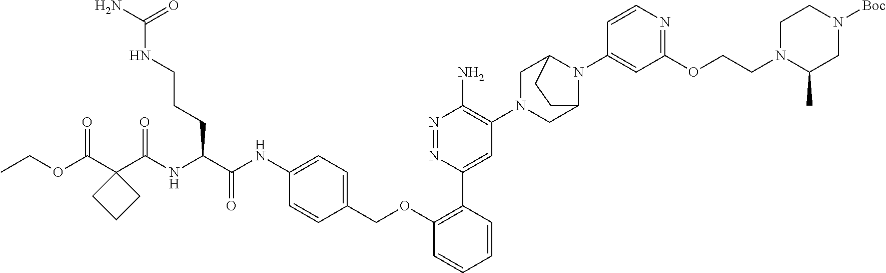 [SMILES: CCOC(=O)C1(C(=O)N[C@@H](CCCNC(N)=O)C(=O)Nc2ccc(COc3ccccc3-c3cc(N4CC5CCC(C4)N5c4ccnc(OCCN5CCN(C(=O)OC(C)(C)C)C[C@H]5C)c4)c(N)nn3)cc2)CCC1]

Under nitrogen, to a solution of tert-butyl (3R)-4-(2-((4-(3-(3-amino-6-(2-hydroxyphenyl)pyridazin-4-yl)-3,8-diazabicyclo[3.2.1]octan-8-yl)pyridin-2-yl)oxy)ethyl)-3-methylpiperazine-1-carboxylate (1.15 g, 1.86 mmol) and Cs₂CO₃ (1.22 g, 3.74 mmol) in DMF (10 mL) was added ethyl (S)-1-((1-((4-(chloromethyl)phenyl)amino)-1-oxo-5-ureidopentan-2-yl)carbamoyl)cyclobutane-1-carboxylate (850 mg, 1.87 mmol) at 0° C. The reaction was stirred at 0° C. for 15 min. EtOAc was added and water was used to wash. Organic layer was dried over anhydrous Na₂SO₄ and concentrated under vacuum. The crude was purified by flash chromatography on silica gel (solvent gradient: 0-15% MeOH in DCM) to yield 690 mg of the title compound as a white solid. LCMS (ESI) [M+H]⁺=1034.

### Step 3: 1-(((2S)-1-((4-((2-(6-Amino-5-(8-(2-(2-((R)-4-(tert-butoxycarbonyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-1-oxo-5-ureidopentan-2-yl)carbamoyl)cyclobutane-1-carboxylic acid

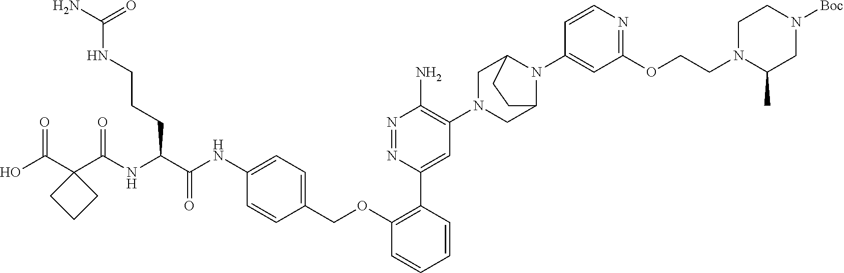 [SMILES: C[C@@H]1CN(C(=O)OC(C)(C)C)CCN1CCOc1cc(N2C3CCC2CN(c2cc(-c4ccccc4OCc4ccc(NC(=O)[C@H](CCCNC(N)=O)NC(=O)C5(C(=O)O)CCC5)cc4)nnc2N)C3)ccn1]

A solution of tert-butyl (3R)-4-(2-((4-(3-(3-amino-6-(2-((4-((S)-2-(1-(ethoxycarbonyl)cyclobutane-1-carboxamido)-5-ureidopentanamido)benzyl)oxy)phenyl)pyridazin-4-yl)-3,8-diazabicyclo[3.2.1]octan-8-yl)pyridin-2-yl)oxy)ethyl)-3-methylpiperazine-1-carboxylate (690 mg, 0.670 mmol) and LiOH·H₂O (84.2 mg, 2.00 mmol) in THF (10 mL) and water (10 mL) was stirred at room temperature for 1 h. The crude was purified by pre-packed C18 column (solvent gradient: 0-100% MeOH in water (0.05% NH₄HCO₃)) to yield 600 mg of the title compound as a white solid. LCMS (ESI) [M+H]=1006.

### Step 4: 1-(((2S)-1-((4-((2-(6-Amino-5-(8-(2-(2-((R)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-1-oxo-5-ureidopentan-2-yl)carbamoyl)cyclobutane-1-carboxylic acid (2,2,2-trifluoroacetic acid salt)

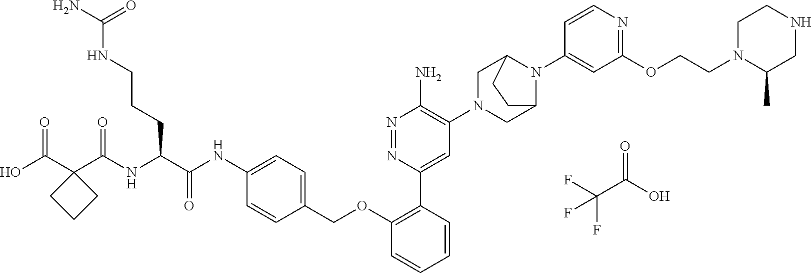 [SMILES: C[C@@H]1CNCCN1CCOc1cc(N2C3CCC2CN(c2cc(-c4ccccc4OCc4ccc(NC(=O)[C@H](CCCNC(N)=O)NC(=O)C5(C(=O)O)CCC5)cc4)nnc2N)C3)ccn1 ; O=C(O)C(F)(F)F]

A solution 1-(((2S)-1-((4-((2-(6-Amino-5-(8-(2-(2-((R)-4-(tert-butoxycarbonyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-1-oxo-5-ureidopentan-2-yl)carbamoyl)cyclobutane-1-carboxylic acid (300 mg, 0.300 mmol) in 5% TFA in HFIP (9 mL) was stirred at room temperature for 1 h. Solvent was evaporated and the crude product was used in the next step directly. LCMS (ESI) [M+H]⁺=906.

### Step 5: 1-(((2S)-1-((4-((2-(6-amino-5-(8-(2-(2-((R)-2-methyl-4-(2-((5-((R)-3-methyl-1-((2S,4R)-2-(((S)-1-(4-(4-methylthiazol-5-yl)phenyl)ethyl)carbamoyl)-4-(phosphonooxy)pyrrolidin-1-yl)-1-oxobutan-2-yl)isoxazol-3-yl)oxy)ethyl)piperazin-1-yl)ethoxy)pyridin-4-yl)-3,8-diazabicyclo[3.2.1]octan-3-yl)pyridazin-3-yl)phenoxy)methyl)phenyl)amino)-1-oxo-5-ureidopentan-2-yl)carbamoyl)cyclobutane-1-carboxylic acid

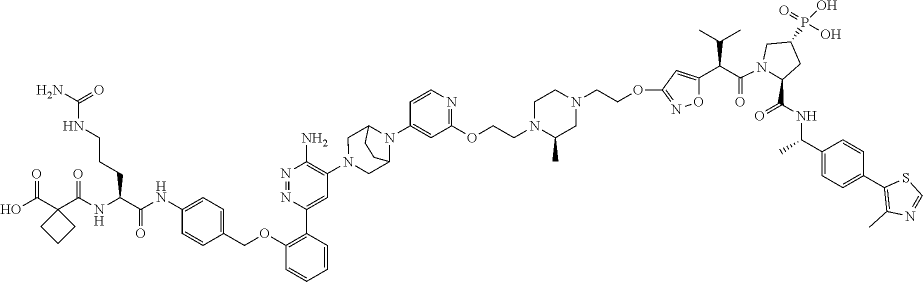 [SMILES: Cc1ncsc1-c1ccc([C@H](C)NC(=O)[C@@H]2C[C@@H](P(=O)(O)O)CN2C(=O)[C@@H](c2cc(OCCN3CCN(CCOc4cc(N5C6CCC5CN(c5cc(-c7ccccc7OCc7ccc(NC(=O)[C@H](CCCNC(N)=O)NC(=O)C8(C(=O)O)CCC8)cc7)nnc5N)C6)ccn4)[C@H](C)C3)no2)C(C)C)cc1]

A solution of 1-(((2S)-1-((4-((2-(6-amino-5-(8-(2-(2-((R)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-1-oxo-5-ureidopentan-2-yl)carbamoyl)cyclobutane-1-carboxylic acid (2,2,2-trifluoroacetic acid salt) (crude from last step), (3R,5S)-1-((R)-3-methyl-2-(3-(2-oxoethoxy)isoxazol-5-yl)butanoyl)-5-(((S)-1-(4-(4-methylthiazol-5-yl)phenyl)ethyl)carbamoyl)pyrrolidin-3-yl dihydrogen phosphate (309 mg, 0.498 mmol) and NaOAc (60.0 mg, 0.731 mmol) in MeOH (10 mL) was stirred at room temperature for 1 h. Then NaBH₃CN (32.0 mg, 0.510 mmol) was added and stirred at room temperature for 1 h. The crude was purified by pre-packed C18 column (0-100% MeOH in water (0.05% NH₄HCO₃)) to yield 324 mg of the title compound as a white solid. LCMS (ESI) [M+H]+=1510.

### Step 6: (3R,5S)-1-((2R)-2-(3-(2-((3R)-4-(2-((4-(3-(3-Amino-6-(2-((4-((S)-2-(1-((5-(2,5-dioxo-2,5-dihydro-1H-pyrrol-1-yl)pentyl)carbamoyl)cyclobutane-1-carboxamido)-5-ureidopentanamido)benzyl)oxy)phenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoyl)-5-(((S)-1-(4-(4-methylthiazol-5-yl)phenyl)ethyl)carbamoyl)pyrrolidin-3-yl dihydrogen phosphate (2,2,2-trifluoroacetic acid salt)

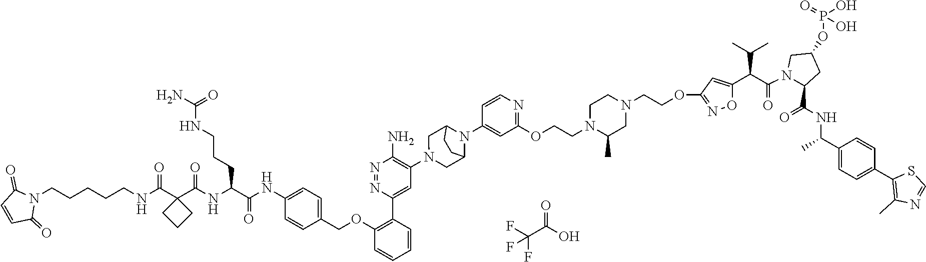 [SMILES: Cc1ncsc1-c1ccc([C@H](C)NC(=O)[C@@H]2C[C@@H](OP(=O)(O)O)CN2C(=O)[C@@H](c2cc(OCCN3CCN(CCOc4cc(N5C6CCC5CN(c5cc(-c7ccccc7OCc7ccc(NC(=O)[C@H](CCCNC(N)=O)NC(=O)C8(C(=O)NCCCCCN9C(=O)C=CC9=O)CCC8)cc7)nnc5N)C6)ccn4)[C@H](C)C3)no2)C(C)C)cc1 ; O=C(O)C(F)(F)F]

A solution of 1-(((2S)-1-((4-((2-(6-amino-5-(8-(2-(2-((R)-2-methyl-4-(2-((5-((R)-3-methyl-1-((2S,4R)-2-(((S)-1-(4-(4-methylthiazol-5-yl)phenyl)ethyl)carbamoyl)-4-(phosphonooxy)pyrrolidin-1-yl)-1-oxobutan-2-yl)isoxazol-3-yl)oxy)ethyl)piperazin-1-yl)ethoxy)pyridin-4-yl)-3,8-diazabicyclo[3.2.1]octan-3-yl)pyridazin-3-yl)phenoxy)methyl)phenyl)amino)-1-oxo-5-ureidopentan-2-yl)carbamoyl)cyclobutane-1-carboxylic acid (324 mg, 0.210 mmol), 1-(5-aminopentyl)-1H-pyrrole-2,5-dione (2,2,2-trifluoroacetic acid salt) (90.9 mg, crude), HATU (106 mg, 0.280 mmol) and DIPEA (277 mg, 2.15 mmol) in DMF was stirred at room temperature for 15 min. The crude was purified by Prep HPLC with the following conditions: Column: Xselect CSH C18 OBD Column 30*150 mm 5 μm; Mobile Phase A: Water (0.05% TFA), Mobile Phase B: ACN; Flow rate: 60 mL/min; Gradient: 19% B to 32% B in 10 min, 32% B; 254/220 nm; R_{T} (min): 10 to yield 173.7 mg of L1-PE-CIDE-2 as a white solid. LCMS (ESI) [M+H]=1674. ¹H NMR (300 MHz, DMSO-d₆, ppm) δ 10.18 (s, 1H), 9.00 (d, J=1.8 Hz, 1H), 8.47 (d, J=7.7 Hz, 1H), 7.91 (d, J=6.4 Hz, 1H), 7.82 (q, J=8.0, 6.7 Hz, 2H), 7.67 (d, J=8.3 Hz, 2H), 7.58 (d, J=7.8 Hz, 2H), 7.53-7.25 (m, 8H), 7.16 (t, J=7.5 Hz, 1H), 6.98 (s, 3H), 6.71 (s, 1H), 6.35 (s, 1H), 6.12 (s, 1H), 6.01 (s, 1H), 5.44 (s, 1H), 5.10 (s, 2H), 4.92 (t, J=7.2 Hz, 1H), 4.83-4.70 (m, 2H), 4.58 (s, 2H), 4.47 (s, 2H), 4.45-4.30 (m, 3H), 3.78-3.67 (m, 11H), 3.40-3.35 (m, 4H), 3.25-3.17 (m, 3H), 3.13-3.05 (m, 5H), 3.00-2.92 (m, 2H), 2.85-2.75 (m, 1H), 2.50-2.28 (m, 9H), 2.10-1.85 (m, 4H), 1.79-1.70 (m, 3H), 1.69 (s, 1H), 1.53-1.30 (m, 9H), 1.27-1.10 (m, 5H), 0.98 (d, J=6.5 Hz, 3H), 0.85-0.75 (m, 3H).

### Example 3

### Synthesis of L1-PE-CIDE-3

### L1-PE-CIDE-3: 4-((S)-6-(Dimethylamino)-2-(1-((5-(2,5-dioxo-2,5-dihydro-1H-pyrrol-1-yl)pentyl)carbamoyl)cyclobutane-1-carboxamido)hexanamido)benzyl (6-(2-hydroxyphenyl)-4-(8-(2-(2-((R)-2-methyl-4-(2-((5-((R)-3-methyl-1-((2S,4R)-2-(((S)-1-(4-(4-methylthiazol-5-yl)phenyl)ethyl)carbamoyl)-4-(phosphonooxy)pyrrolidin-1-yl)-1-oxobutan-2-yl)isoxazol-3-yl)oxy)ethyl)piperazin-1-yl)ethoxy)pyridin-4-yl)-3,8-diazabicyclo[3.2.1]octan-3-yl)pyridazin-3-yl)carbamate (2,2,2-trifluoroacetic acid salt)

### Step 1: 1-(((2S)-6-(Dimethylamino)-1-((4-((((6-(2-hydroxyphenyl)-4-(8-(2-(2-((R)-2-methyl-4-(2-((5-((R)-3-methyl-1-((2S,4R)-2-(((S)-1-(4-(4-methylthiazol-5-yl)phenyl)ethyl)carbamoyl)-4-(phosphonooxy)pyrrolidin-1-yl)-1-oxobutan-2-yl)isoxazol-3-yl)oxy)ethyl)piperazin-1-yl)ethoxy)pyridin-4-yl)-3,8-diazabicyclo[3.2.1]octan-3-yl)pyridazin-3-yl)carbamoyl)oxy)methyl)phenyl)amino)-1-oxohexan-2-yl)carbamoyl)cyclobutane-1-carboxylic acid

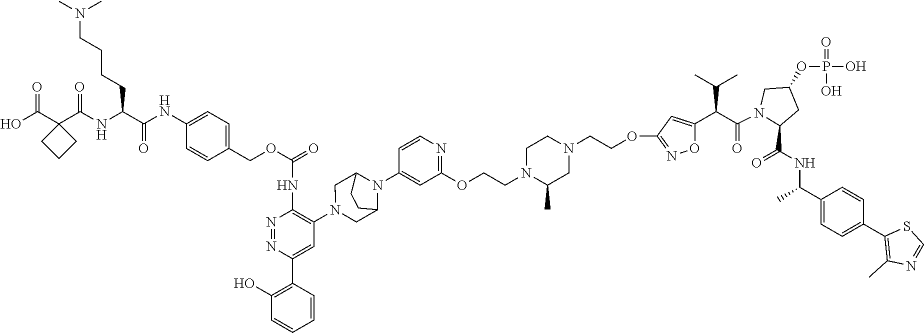 [SMILES: Cc1ncsc1-c1ccc([C@H](C)NC(=O)[C@@H]2C[C@@H](OP(=O)(O)O)CN2C(=O)[C@@H](c2cc(OCCN3CCN(CCOc4cc(N5C6CCC5CN(c5cc(-c7ccccc7O)nnc5NC(=O)OCc5ccc(NC(=O)[C@H](CCCCN(C)C)NC(=O)C7(C(=O)O)CCC7)cc5)C6)ccn4)[C@H](C)C3)no2)C(C)C)cc1]

Under nitrogen, a solution of 1-(((2S)-6-(dimethylamino)-1-((4-((((6-(2-hydroxyphenyl)-4-(8-(2-(2-((R)-2-methylpiperazin-1-yl)ethoxy)pyridin-4-yl)-3,8-diazabicyclo[3.2.1]octan-3-yl)pyridazin-3-yl)carbamoyl)oxy)methyl)phenyl)amino)-1-oxohexan-2-yl)carbamoyl)cyclobutane-1-carboxylic acid (50.0 mg, 0.0527 mmol, same INT as described in Example 3a, (3R,5S)-1-((R)-3-methyl-2-(3-(2-oxoethoxy)isoxazol-5-yl)butanoyl)-5-(((S)-1-(4-(4-methylthiazol-5-yl)phenyl)ethyl)carbamoyl)pyrrolidin-3-yl dihydrogen phosphate (26.0 mg, 0.0419 mmol), HOAc (9.5 mg, 0.157 mmol) in MeOH (3 mL) and DCM (1 ml) was stirred at 30° C. for 1 h. Then NaBH₃CN (6.6 mg, 0.105 mmol) was added and stirred at 30° C. for 0.5 h. The reaction solution was concentrated under vacuum. The residue was purified by pre-packed C18 column (solvent gradient: 0-100% CH₃OH in water (0.05% NH₄HCO₃)) to yield 23.0 mg of the title compound as a white solid. LCMS (ESI) [M+H]⁺=1553.

### Step 2: 4-((S)-6-(Dimethylamino)-2-(1-((5-(2,5-dioxo-2,5-dihydro-1H-pyrrol-1-yl)pentyl)carbamoyl)cyclobutane-1-carboxamido)hexanamido)benzyl (6-(2-hydroxyphenyl)-4-(8-(2-(2-((R)-2-methyl-4-(2-((5-((R)-3-methyl-1-((2S,4R)-2-(((S)-1-(4-(4-methylthiazol-5-yl)phenyl)ethyl)carbamoyl)-4-(phosphonooxy)pyrrolidin-1-yl)-1-oxobutan-2-yl)isoxazol-3-yl)oxy)ethyl)piperazin-1-yl)ethoxy)pyridin-4-yl)-3,8-diazabicyclo[3.2.1]octan-3-yl)pyridazin-3-yl)carbamate (2,2,2-trifluoroacetic acid salt)

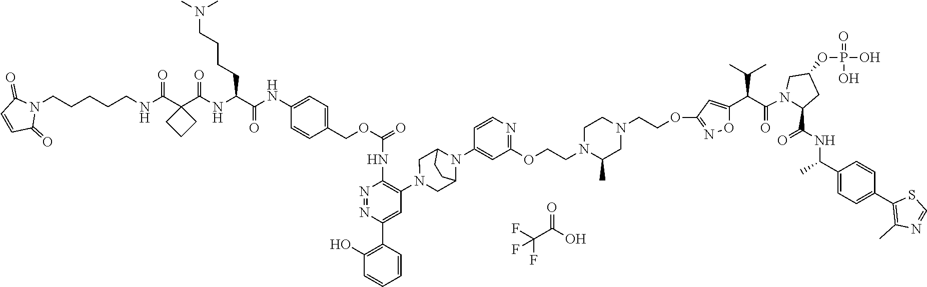 [SMILES: Cc1ncsc1-c1ccc([C@H](C)NC(=O)[C@@H]2C[C@@H](OP(=O)(O)O)CN2C(=O)[C@@H](c2cc(OCCN3CCN(CCOc4cc(N5C6CCC5CN(c5cc(-c7ccccc7O)nnc5NC(=O)OCc5ccc(NC(=O)[C@H](CCCCN(C)C)NC(=O)C7(C(=O)NCCCCCN8C(=O)C=CC8=O)CCC7)cc5)C6)ccn4)[C@H](C)C3)no2)C(C)C)cc1 ; O=C(O)C(F)(F)F]

A solution of 1-(((2S)-6-(dimethylamino)-1-((4-((((6-(2-hydroxyphenyl)-4-(8-(2-(2-((R)-2-methyl-4-(2-((5-((R)-3-methyl-1-((2S,4R)-2-(((S)-1-(4-(4-methylthiazol-5-yl)phenyl)ethyl)carbamoyl)-4-(phosphonooxy)pyrrolidin-1-yl)-1-oxobutan-2-yl)isoxazol-3-yl)oxy)ethyl)piperazin-1-yl)ethoxy)pyridin-4-yl)-3,8-diazabicyclo[3.2.1]octan-3-yl)pyridazin-3-yl)carbamoyl)oxy)methyl)phenyl)amino)-1-oxohexan-2-yl)carbamoyl)cyclobutane-1-carboxylic acid (23.0 mg, 0.0148 mmol), 1-(5-aminopentyl)-1H-pyrrole-2,5-dione (2,2,2-trifluoroacetic acid salt) (5.5 mg, crude), HATU (7.3 mg, 0.0192 mmol) and DIPEA (19.8 mg, 0.153 mmol) in DMF (1 mL) was stirred at room temperature for 10 min. The crude was purified by Prep-HPLC with the following conditions: Column: Xselect CSH C18 OBD Column 30*150 mm 5 μm; Mobile Phase A: Water (0.05% TFA), Mobile Phase B: ACN; Flow rate: 60 mL/min; Gradient: 18% B to 30% B in 12 min, 30% B; Wavelength: 254/220 nm; R_{T}(min): 11 to yield 8.7 mg of L1-PE-CIDE-3 as a white solid. LCMS (ESI) [M+H]+=1717. ¹H NMR (300 MHz, DMSO-d₆, ppm) δ 10.15 (s, 1H), 10.07 (s, 1H), 9.37 (br, 1H), 8.95 (s, 1H), 8.48 (d, J=7.6 Hz, 1H), 8.00-7.80 (m, 4H), 7.66-7.57 (m, 3H), 7.49-7.28 (m, 6H), 7.02-6.89 (m, 4H), 6.78 (d, J=7.1 Hz, 1H), 6.44 (s, 1H), 6.10 (s, 1H), 5.10 (s, 2H), 4.89 (t, J=6.9 Hz, 1H), 4.80 (s, 1H), 4.73 (s, 2H), 4.50-4.30 (m, 6H), 3.85-3.63 (m, 9H), 3.52-3.45 (m, 3H), 3.35 (t, J=7.0 Hz, 3H), 3.20-2.98 (m, 8H), 2.90-2.70 (m, 8H), 2.48-2.34 (m, 9H), 1.93 (s, 4H), 1.83-1.55 (m, 6H), 1.49-1.32 (m, 8H), 1.28-1.13 (m, 6H), 0.94 (d, J=6.4 Hz, 3H), 0.85-0.74 (m, 3H).

### Example 3a

### 4-((S)-6-(Dimethylamino)-2-(1-((5-((2-(2,5-dioxo-2,5-dihydro-1H-pyrrol-1-yl)ethyl)(methyl)amino)pentyl)carbamoyl)cyclobutane-1-carboxamido)hexanamido)benzyl (4-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)-6-(2-hydroxyphenyl)pyridazin-3-yl)carbamate (2,2,2-trifluoroacetic acid salt)

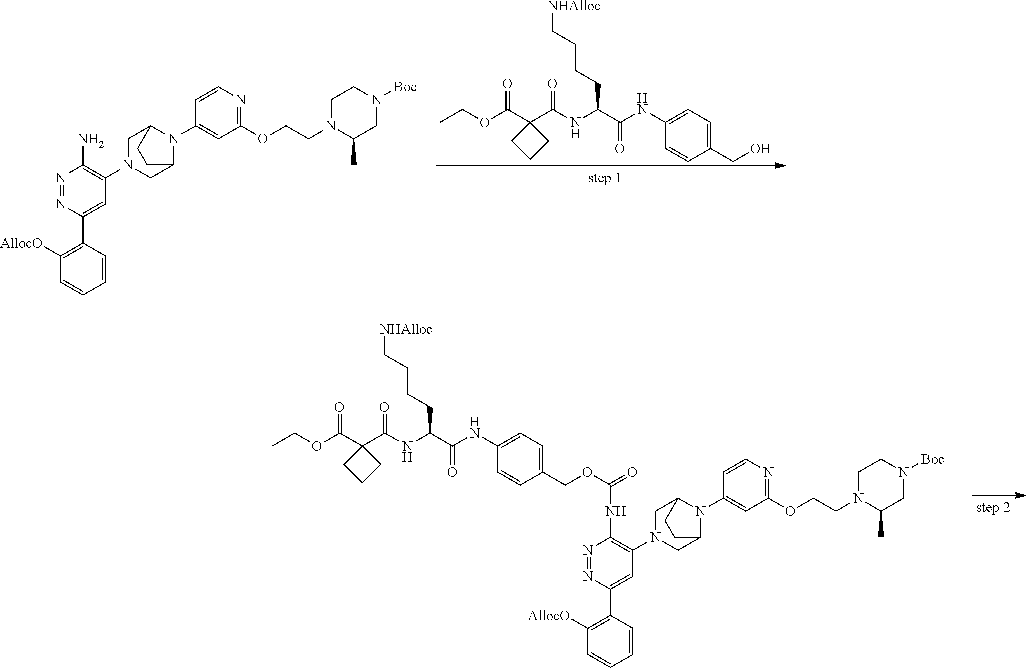

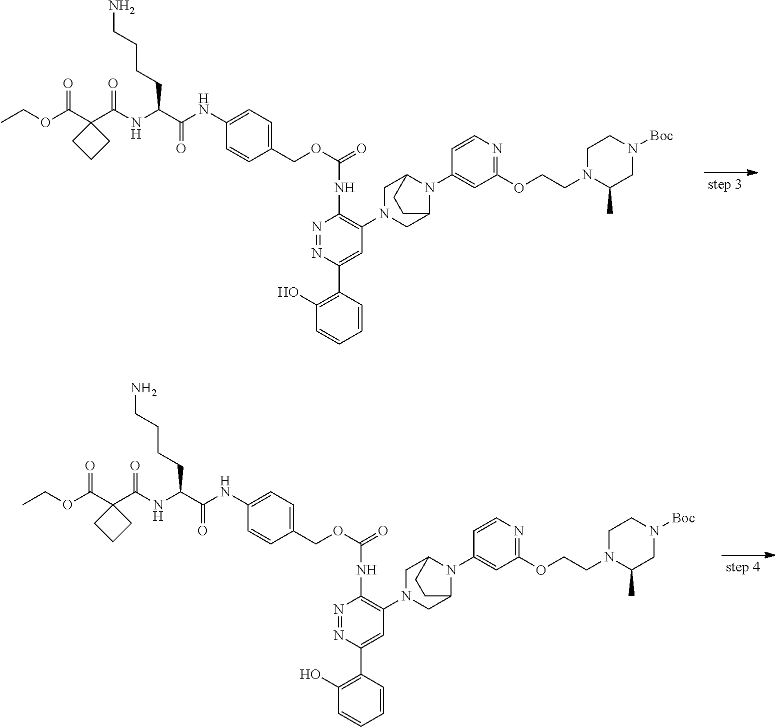 [SMILES: CCOC(=O)C1(C(=O)N[C@@H](CCCCN)C(=O)Nc2ccc(COC(=O)Nc3nnc(-c4ccccc4O)cc3N3CC4CCC(C3)N4c3ccnc(OCCN4CCN(C(=O)OC(C)(C)C)C[C@H]4C)c3)cc2)CCC1 ; CCOC(=O)C1(C(=O)N[C@@H](CCCCN)C(=O)Nc2ccc(COC(=O)Nc3nnc(-c4ccccc4O)cc3N3CC4CCC(C3)N4c3ccnc(OCCN4CCN(C(=O)OC(C)(C)C)C[C@H]4C)c3)cc2)CCC1]

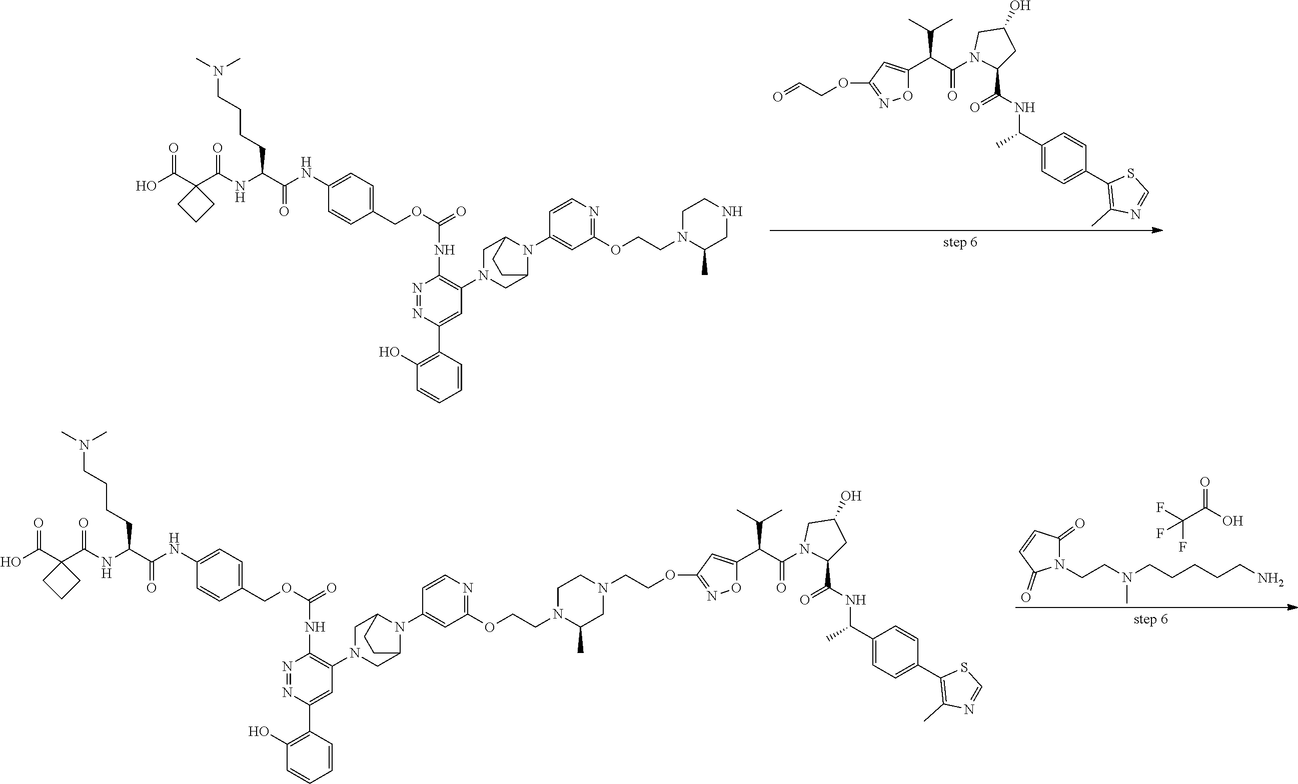

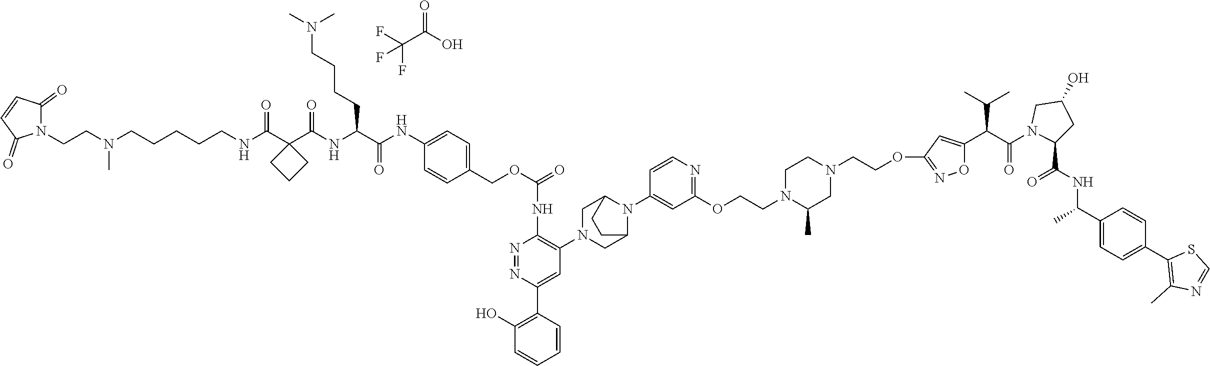 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7O)nnc5NC(=O)OCc5ccc(NC(=O)[C@H](CCCCN(C)C)NC(=O)C7(C(=O)NCCCCCN(C)CCN8C(=O)C=CC8=O)CCC7)cc5)C6)ccn4)[C@H](C)C3)no2)C(C)C)cc1 ; O=C(O)C(F)(F)F]

### Step 1: tert-Butyl (3R)-4-(2-((4-(3-(3-((((4-((S)-6-(((allyloxy)carbonyl)amino)-2-(1-(ethoxycarbonyl)cyclobutane-1-carboxamido)hexanamido)benzyl)oxy)carbonyl)amino)-6-(2-(((allyloxy)carbonyl)oxy)phenyl)pyridazin-4-yl)-3,8-diazabicyclo[3.2.1]octan-8-yl)pyridin-2-yl)oxy)ethyl)-3-methylpiperazine-1-carboxylate

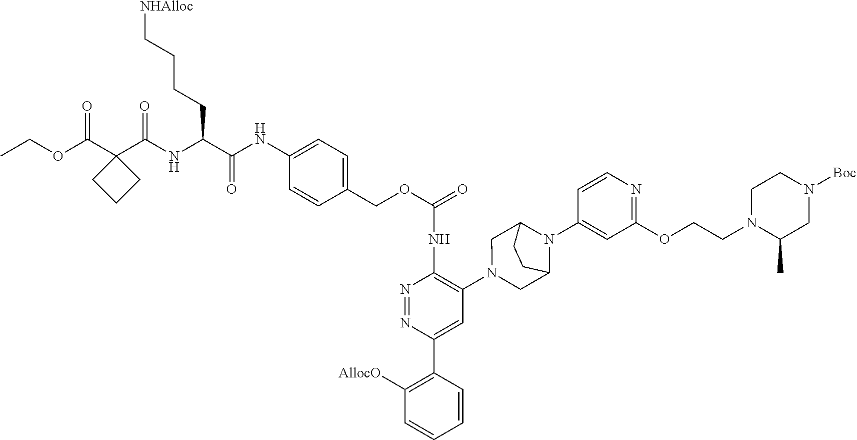

Under nitrogen, to a solution of tert-butyl (3R)-4-(2-((4-(3-(6-(2-(((allyloxy)carbonyl)oxy)phenyl)-3-aminopyridazin-4-yl)-3,8-diazabicyclo[3.2.1]octan-8-yl)pyridin-2-yl)oxy)ethyl)-3-methylpiperazine-1-carboxylate (500 mg, 0.714 mmol), ethyl (S)-1-((6-(((allyloxy)carbonyl)amino)-1-((4-(hydroxymethyl)phenyl)amino)-1-oxohexan-2-yl)carbamoyl)cyclobutane-1-carboxylate (1.05 g, 2.15 mmol) and DIPEA (554 mg, 4.29 mmol) in DCM (15 mL) was added a solution of triphosgene (109 mg, 0.367 mmol) in DCM (1 mL) at 0° C. The reaction was stirred at room temperature for 2 h. The solvent was concentrated under vacuum. The residue was purified by pre-packed C18 column (solvent gradient: 0-100% CH₃CN in water (0.05% NH₄HCO₃)) to yield 480 mg (55% yield) of the title compound as a white solid. LC-MS: (ESI, m/z): [M+H]=1216.

### Step 2: tert-Butyl (3R)-4-(2-((4-(3-(3-((((4-((S)-6-amino-2-(1-(ethoxycarbonyl)cyclobutane-1-carboxamido)hexanamido)benzyl)oxy)carbonyl)amino)-6-(2-hydroxyphenyl)pyridazin-4-yl)-3,8-diazabicyclo[3.2.1]octan-8-yl)pyridin-2-yl)oxy)ethyl)-3-methylpiperazine-1-carboxylate

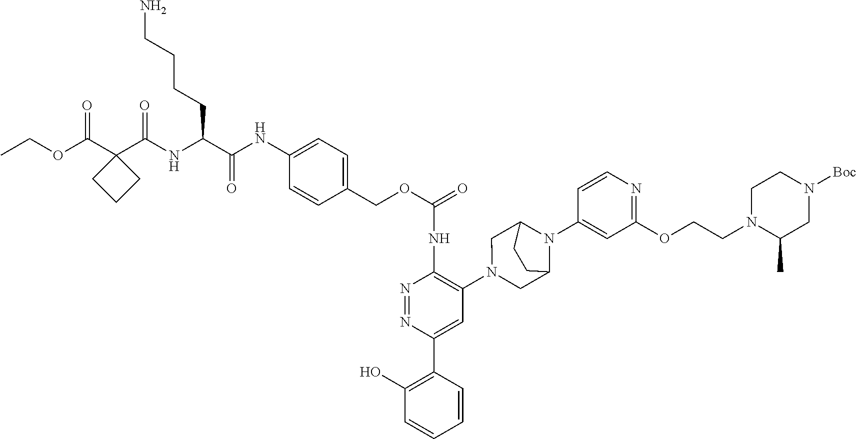 [SMILES: CCOC(=O)C1(C(=O)N[C@@H](CCCCN)C(=O)Nc2ccc(COC(=O)Nc3nnc(-c4ccccc4O)cc3N3CC4CCC(C3)N4c3ccnc(OCCN4CCN(C(=O)OC(C)(C)C)C[C@H]4C)c3)cc2)CCC1]

Under nitrogen, to a solution of tert-butyl (3R)-4-(2-((4-(3-(3-((((4-((S)-6-(((allyloxy)carbonyl)amino)-2-(1-(ethoxycarbonyl)cyclobutane-1-carboxamido)hexanamido)benzyl)oxy)carbonyl)amino)-6-(2-(((allyloxy)carbonyl)oxy)phenyl)pyridazin-4-yl)-3,8-diazabicyclo[3.2.1]octan-8-yl)pyridin-2-yl)oxy)ethyl)-3-methylpiperazine-1-carboxylate (400 mg, 0.329 mmol), pyrrolidine (234 mg, 3.29 mmol) in DCM (5 mL) was added Pd(PPh₃)₄ (76.0 mg, 0.0658 mmol) at room temperature. The reaction was stirred at room temperature for 1 h. The reaction mixture was diluted with DCM (100 mL) and washed with water. The organic layer was dried over anhydrous sodium sulfate and concentrated under vacuum to afford 390 mg (crude) of the title compound as a yellow solid. LC-MS: (ESI, m/z): [M+H]=1048.

### Step 3: tert-Butyl (3R)-4-(2-((4-(3-(3-((((4-((S)-6-(dimethylamino)-2-(1-(ethoxycarbonyl)cyclobutane-1-carboxamido)hexanamido)benzyl)oxy)carbonyl)amino)-6-(2-hydroxyphenyl)pyridazin-4-yl)-3,8-diazabicyclo[3.2.1]octan-8-yl)pyridin-2-yl)oxy)ethyl)-3-methylpiperazine-1-carboxylate

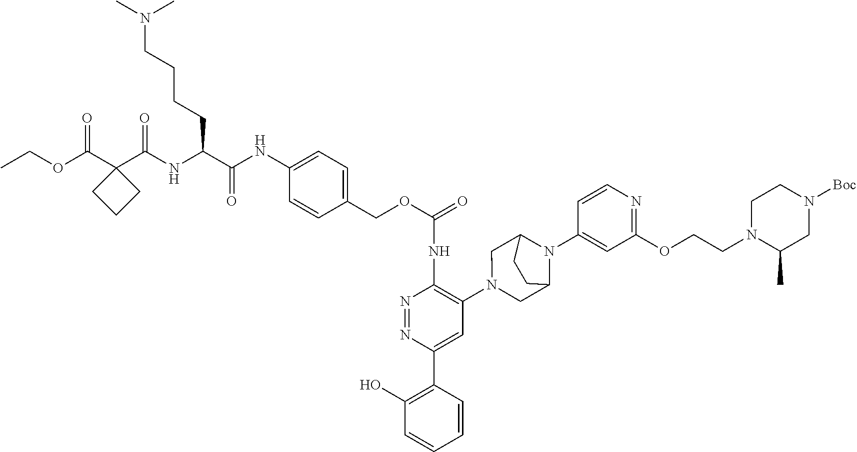 [SMILES: CCOC(=O)C1(C(=O)N[C@@H](CCCCN(C)C)C(=O)Nc2ccc(COC(=O)Nc3nnc(-c4ccccc4O)cc3N3CC4CCC(C3)N4c3ccnc(OCCN4CCN(C(=O)OC(C)(C)C)C[C@H]4C)c3)cc2)CCC1]

A solution of tert-butyl (3R)-4-(2-((4-(3-(3-((((4-((S)-6-amino-2-(1-(ethoxycarbonyl)cyclobutane-1-carboxamido)hexanamido)benzyl)oxy)carbonyl)amino)-6-(2-hydroxyphenyl)pyridazin-4-yl)-3,8-diazabicyclo[3.2.1]octan-8-yl)pyridin-2-yl)oxy)ethyl)-3-methylpiperazine-1-carboxylate (465 mg, 0.440 mmol) and 37% aqueous CH₂O (465 mg, 5.73 mmol) in CH₃OH (5 mL) was stirred at room temperature for 1 h. Then NaBH₃CN (140 mg, 2.22 mmol) was added and stirred at room temperature for 1 hour. The reaction was quenched by water (0.2 mL). The residue was purified by pre-packed C18 column (solvent gradient: 0-100% MeOH in water (0.05% NH₄HCO₃)) to yield 260 mg (54% yield) of the title compound as a white solid. LC-MS: (ESI, m z): [M]⁺=1077.

### Step 4: 1-(((2S)-6-(Dimethylamino)-1-((4-((((6-(2-hydroxyphenyl)-4-(8-(2-(2-((R)-2-methylpiperazin-1-yl)ethoxy)pyridin-4-yl)-3,8-diazabicyclo[3.2.1]octan-3-yl)pyridazin-3-yl)carbamoyl)oxy)methyl)phenyl)amino)-1-oxohexan-2-yl)carbamoyl)cyclobutane-1-carboxylic acid

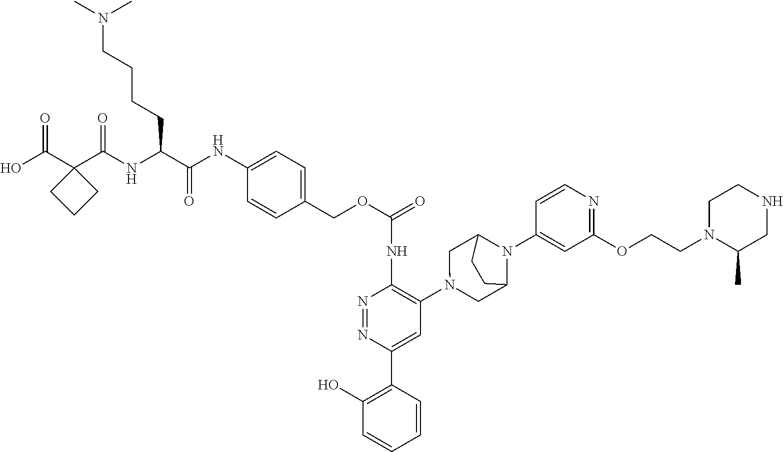 [SMILES: C[C@@H]1CNCCN1CCOc1cc(N2C3CCC2CN(c2cc(-c4ccccc4O)nnc2NC(=O)OCc2ccc(NC(=O)[C@H](CCCCN(C)C)NC(=O)C4(C(=O)O)CCC4)cc2)C3)ccn1]

A solution of tert-butyl (3R)-4-(2-((4-(3-(3-((((4-((S)-6-(dimethylamino)-2-(1-(ethoxycarbonyl)cyclobutane-1-carboxamido)hexanamido)benzyl)oxy)carbonyl)amino)-6-(2-hydroxyphenyl)pyridazin-4-yl)-3,8-diazabicyclo[3.2.1]octan-8-yl)pyridin-2-yl)oxy)ethyl)-3-methylpiperazine-1-carboxylate (75.0 mg, 0.0697 mmol) and LiOH·H₂O (8.4 mg, 0.210 mmol) in THF (5 mL) and water (5 mL) was stirred at room temperature for 1 h. The solvent was concentrated under vacuum. Then 5% TFA in HFIP (3 mL) was added and the solution was stirred at room temperature for 1 h. The solvent was concentrated under vacuum. The residue was purified by pre-packed C18 column (solvent gradient: 0-100% MeOH in water (0.05% NH₄HCO₃)) to yield 55.0 mg (83% yield) of the title compound as a white solid. LC-MS: (ESI, m z): [M]⁺=949.

### Step 5: 1-(((2S)-6-(Dimethylamino)-1-((4-((((4-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)-6-(2-hydroxyphenyl)pyridazin-3-yl)carbamoyl)oxy)methyl)phenyl)amino)-1-oxohexan-2-yl)carbamoyl)cyclobutane-1-carboxylic acid

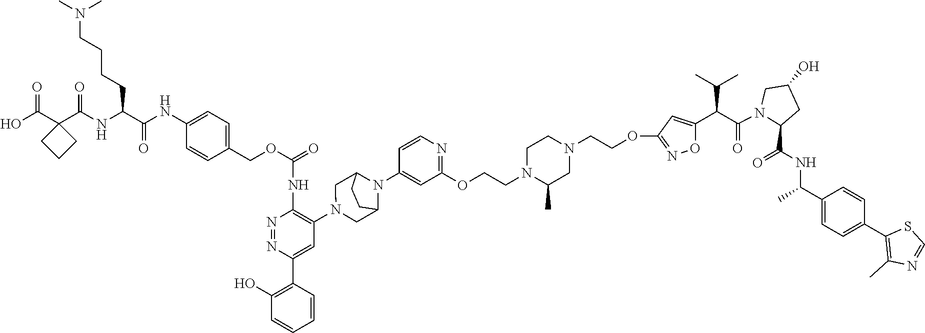 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7O)nnc5NC(=O)OCc5ccc(NC(=O)[C@H](CCCCN(C)C)NC(=O)C7(C(=O)O)CCC7)cc5)C6)ccn4)[C@H](C)C3)no2)C(C)C)cc1]

Under nitrogen, a solution of 1-(((2S)-6-(dimethylamino)-1-((4-((((6-(2-hydroxyphenyl)-4-(8-(2-(2-((R)-2-methylpiperazin-1-yl)ethoxy)pyridin-4-yl)-3,8-diazabicyclo[3.2.1]octan-3-yl)pyridazin-3-yl)carbamoyl)oxy)methyl)phenyl)amino)-1-oxohexan-2-yl)carbamoyl)cyclobutane-1-carboxylic acid (20.0 g, 0.0211 mmol), (2S,4R)-4-hydroxy-1-((R)-3-methyl-2-(3-(2-oxoethoxy)isoxazol-5-yl)butanoyl)-N—((S)-1-(4-(4-methylthiazol-5-yl)phenyl)ethyl)pyrrolidine-2-carboxamide (19.4 mg, 0.0359 mmol) and NaOAc (4.0 mg, 0.0488 mmol) in methyl alcohol (1.5 mL) was stirred at room temperature for 1 hour. Then NaBH₃CN (5.2 mg, 0.0827 mol) was added and stirred at room temperature for 3 h. The reaction solution was concentrated under vacuum. The residue was purified by pre-packed C18 column (solvent gradient: 0-100% CH₃OH in water (0.05% NH₄HCO₃)) to yield 18 mg (58% yield) of the title compound as a white solid. LC-MS: (ESI, m/z): [M+H]⁺=1473.

### Step 6: 4-((S)-6-(Dimethylamino)-2-(1-((5-((2-(2,5-dioxo-2,5-dihydro-1H-pyrrol-1-yl)ethyl)(methyl)amino)pentyl)carbamoyl)cyclobutane-1-carboxamido)hexanamido)benzyl (4-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)-6-(2-hydroxyphenyl)pyridazin-3-yl)carbamate (2,2,2-trifluoroacetic acid salt)

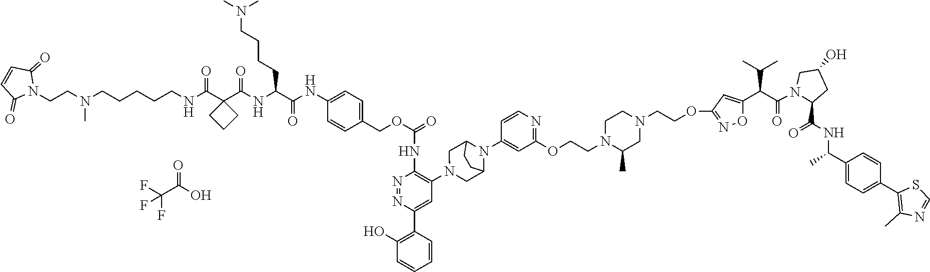 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7O)nnc5NC(=O)OCc5ccc(NC(=O)[C@H](CCCCN(C)C)NC(=O)C7(C(=O)NCCCCCN(C)CCN8C(=O)C=CC8=O)CCC7)cc5)C6)ccn4)[C@H](C)C3)no2)C(C)C)cc1 ; O=C(O)C(F)(F)F]

To a solution of 1-(((2S)-6-(dimethylamino)-1-((4-((((4-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)-6-(2-hydroxyphenyl)pyridazin-3-yl)carbamoyl)oxy)methyl)phenyl)amino)-1-oxohexan-2-yl)carbamoyl)cyclobutane-1-carboxylic acid (40.0 mg, 0.0272 mmol), 1-(2-((5-aminopentyl)(methyl)amino)ethyl)-1H-pyrrole-2,5-dione (2,2,2-trifluoroacetic acid salt) (9.70 mg, crude) and DIPEA (35.1 mg, 0.272 mmol) in DMF (1 mL) was added HATU (13.5 mg, 0.0355 mmol) at room temperature. The reaction was stirred at room temperature for 10 min. The crude was purified by Prep-HPLC with following conditions: Column: XSelect CSH Fluoro Phenyl, 30*150 mm, 5 μm; Mobile Phase A: Water (0.05% TFA), Mobile Phase B: ACN; Flow rate: 60 mL/min; Gradient: 15% B to 20% B in 10 min; Wave Length: 254/220 nm; R_{T}(min): 10.5 to yield 13.0 mg (28.3% yield) of the target compound as a yellow solid. LC-MS: (ESI, m/z): [M+H]=1808. ¹H NMR (300 MHz, DMSO-d₆, ppm) δ 10.09 (s, 1H), 9.97 (s, 1H), 9.50 (s, 2H), 8.88 (d, J=1.3 Hz, 1H), 8.30 (d, J=7.7 Hz, 1H), 8.00-7.80 (m, 4H), 7.58-7.50 (m, 3H), 7.39-7.16 (m, 7H), 6.96 (s, 2H), 6.84 (ddd, J=11.0, 7.0, 2.3 Hz, 2H), 6.65 (d, J=6.6 Hz, 1H), 6.29 (s, 1H), 6.00 (s, 1H), 5.01 (s, 2H), 4.95-4.80 (m, 1H), 4.59 (s, 2H), 4.37 (s, 2H), 4.35-4.21 (m, 4H), 4.21-4.15 (m, 2H), 3.59-3.48 (m, 6H), 3.40-3.30 (m, 4H), 3.27-2.97 (m, 14H), 2.89 (s, 4H), 2.70-2.60 (m, 9H), 2.45-2.34 (m, 4H), 2.30-2.19 (m, 2H), 2.17-2.03 (m, 2H), 2.05-1.83 (m, 5H), 1.83-1.66 (m, 5H), 1.66-1.49 (m, 4H), 1.34 (d, J=7.1 Hz, 3H), 1.25 (d, J=7.0 Hz, 3H), 1.20-1.10 (m, 6H), 0.85 (d, J=6.6 Hz, 3H), 0.69 (dd, J=10.7, 6.6 Hz, 3H).

### Example 4

### Synthesis of L1-PP-CIDE-1

### L1-PP-CIDE-1: 6-(8-Chloro-4-(2-(dimethylamino)ethyl)-2-((tetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (2,2,2-trifluoroacetic acid salt)

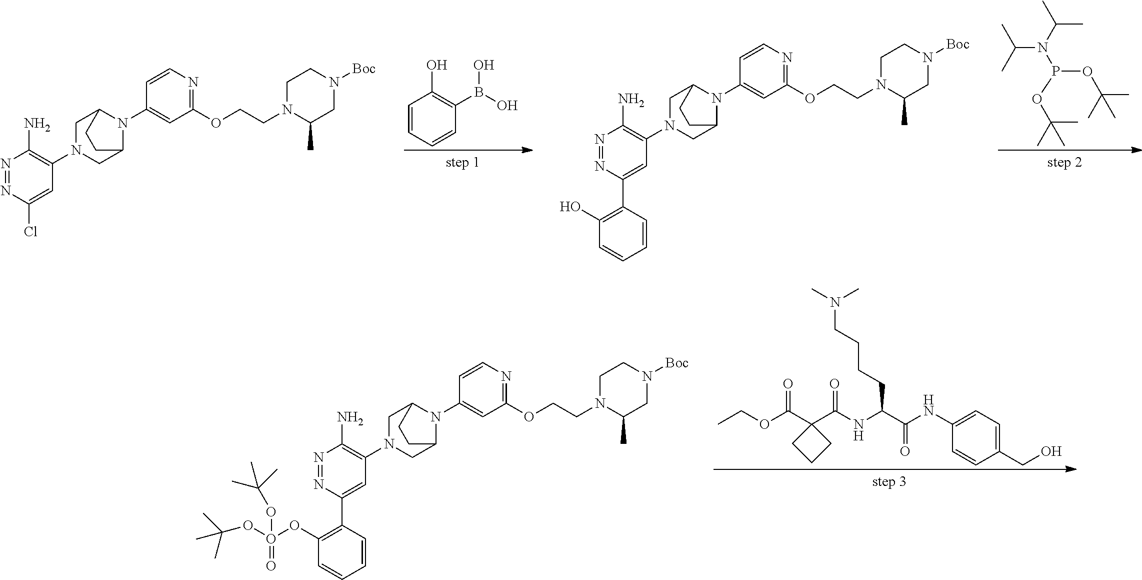

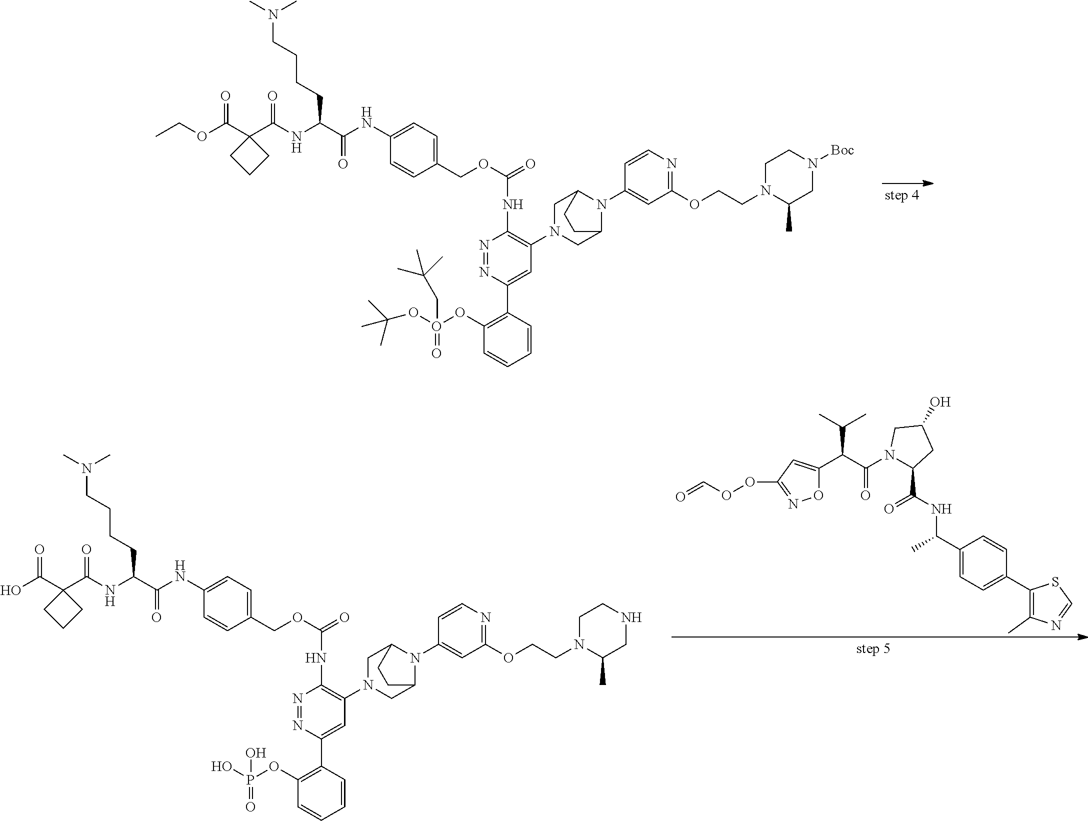

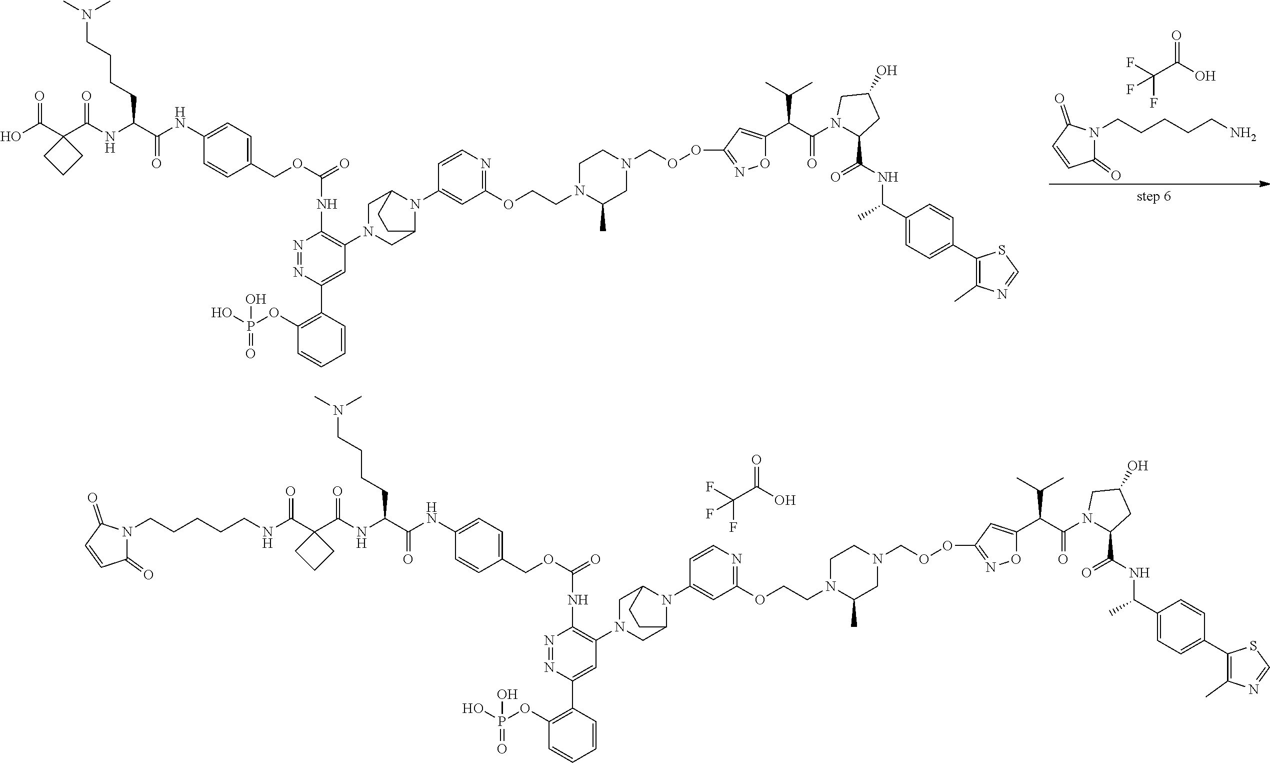

### Step 1: tert-Butyl (3R)-4-(2-((4-(3-(3-amino-6-(2-hydroxyphenyl)pyridazin-4-yl)-3,8-diazabicyclo[3.2.1]octan-8-yl)pyridin-2-yl)oxy)ethyl)-3-methylpiperazine-1-carboxylate

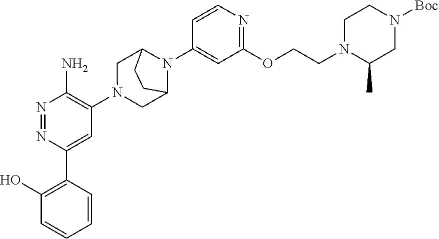 [SMILES: C[C@@H]1CN(C(=O)OC(C)(C)C)CCN1CCOc1cc(N2C3CCC2CN(c2cc(-c4ccccc4O)nnc2N)C3)ccn1]

Under nitrogen, a solution of tert-butyl (3R)-4-(2-((4-(3-(3-amino-6-chloropyridazin-4-yl)-3,8-diazabicyclo[3.2.1]octan-8-yl)pyridin-2-yl)oxy)ethyl)-3-methylpiperazine-1-carboxylate (3.00 g, 5.37 mmol, provided by Genentech), 2-hydroxyphenylboronic acid (0.890 g, 6.46 mmol), Pd(PPh₃)₄ (1.24 g, 1.08 mmol) and K₂CO₃ (2.23 g, 16.1 mmol) in dioxane (37.5 mL) and H₂O (7.5 mL) was stirred at 100° C. for 1 h. The reaction was diluted with water and extracted with DCM. The combined organic layer was dried over anhydrous Na₂SO₄ and concentrated under reduced pressure. The obtained crude product was purified by flash chromatography on silica gel (solvent gradient: 0-10% MeOH/DCM) to afford 2.26 g (68% yield) of the title compound as a yellow solid. LCMS (ESI, m/z): [M+H]=617.

### Step 2: tert-Butyl (3R)-4-(2-((4-(3-(3-amino-6-(2-((di-tert-butoxyphosphoryl)oxy)phenyl)pyridazin-4-yl)-3,8-diazabicyclo[3.2.1]octan-8-yl)pyridin-2-yl)oxy)ethyl)-3-methylpiperazine-1-carboxylate

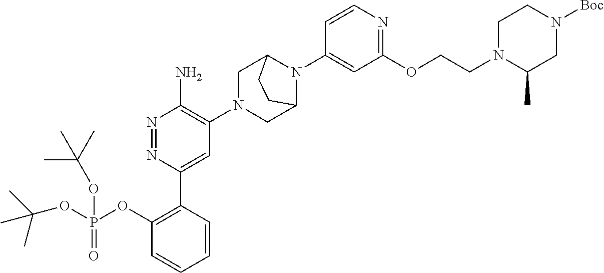 [SMILES: C[C@@H]1CN(C(=O)OC(C)(C)C)CCN1CCOc1cc(N2C3CCC2CN(c2cc(-c4ccccc4OP(=O)(OC(C)(C)C)OC(C)(C)C)nnc2N)C3)ccn1]

Under nitrogen, to a solution of tert-butyl (3R)-4-(2-((4-(3-(3-amino-6-(2-hydroxyphenyl)pyridazin-4-yl)-3,8-diazabicyclo[3.2.1]octan-8-yl)pyridin-2-yl)oxy)ethyl)-3-methylpiperazine-1-carboxylate (500 mg, 0.811 mmol) and 1H-tetrazole (5.4 mL, 0.45 M in ACN) in THF (10 mL) was added di-tert-butyl diisopropylphosphoramidite (1.35 g, 4.86 mmol) dropwise at 0° C. The reaction was stirred at 0° C. for 1 h. Then t-BuOOH (5M in decane) (0.64 mL, 3.20 mmol) was added at −30° C. and stirred at −30° C. for 30 min. Then the mixture was stirred at room temperature overnight. The reaction mixture was poured into aqueous NaHSO₃ solution and extracted with EtOAc. The combined organic layer was washed with water, brine, dried over anhydrous Na₂SO₄ and concentrated under reduced pressure. The obtained crude product was purified by pre-packed C18 column (solvent gradient: 0-100% ACN in water (0.05% NH₄HCO₃)) to yield 140 mg (21% yield) of the title compound as a yellow oil. LCMS (ESI, m/z): [M+H]⁺=809.

### Step 3: tert-Butyl (3R)-4-(2-((4-(3-(6-(2-((di-tert-butoxyphosphoryl)oxy)phenyl)-3-((((4-((S)-6-(dimethylamino)-2-(1-(ethoxycarbonyl)cyclobutane-1-carboxamido)hexanamido)benzyl)oxy)carbonyl)amino)pyridazin-4-yl)-3,8-diazabicyclo[3.2.1]octan-8-yl)pyridin-2-yl)oxy)ethyl)-3-methylpiperazine-1-carboxylate

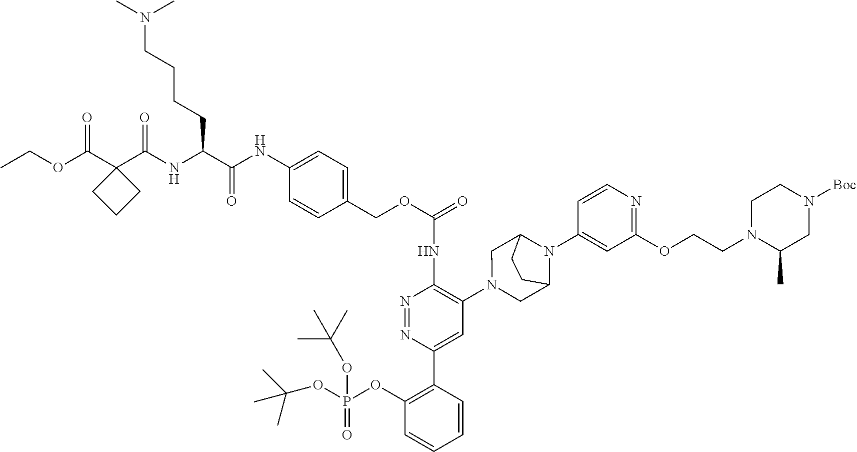 [SMILES: CCOC(=O)C1(C(=O)N[C@@H](CCCCN(C)C)C(=O)Nc2ccc(COC(=O)Nc3nnc(-c4ccccc4OP(=O)(OC(C)(C)C)OC(C)(C)C)cc3N3CC4CCC(C3)N4c3ccnc(OCCN4CCN(C(=O)OC(C)(C)C)C[C@H]4C)c3)cc2)CCC1]

Under nitrogen, to a solution of tert-butyl (3R)-4-(2-((4-(3-(3-amino-6-(2-((di-tert-butoxyphosphoryl)oxy)phenyl)pyridazin-4-yl)-3,8-diazabicyclo[3.2.1]octan-8-yl)pyridin-2-yl)oxy)ethyl)-3-methylpiperazine-1-carboxylate (120 mg, 0.148 mmol), ethyl (S)-1-((6-(dimethylamino)-1-((4-(hydroxymethyl)phenyl)amino)-1-oxohexan-2-yl)carbamoyl)cyclobutane-1-carboxylate (225 mg, 0.518 mmol) and DIPEA (303 mg, 2.34 mmol) in THF (5 mL) was added triphosgene (79.8 mg, 0.269 mmol) at 0° C. The reaction was stirred at room temperature for 30 min. Then the reaction mixture was purified by pre-packed C18 column (solvent gradient: 0-100% MeOH in water (0.05% NH₄HCO₃)) to yield 35 mg of the title compound as a yellow solid. LCMS (ESI, m/z): [M+H]=1269.

### Step 4: 1-(((2S)-6-(Dimethylamino)-1-((4-((((4-(8-(2-(2-((R)-2-methylpiperazin-1-yl)ethoxy)pyridin-4-yl)-3,8-diazabicyclo[3.2.1]octan-3-yl)-6-(2-(phosphonooxy)phenyl)pyridazin-3-yl)carbamoyl)oxy)methyl)phenyl)amino)-1-oxohexan-2-yl)carbamoyl)cyclobutane-1-carboxylic acid

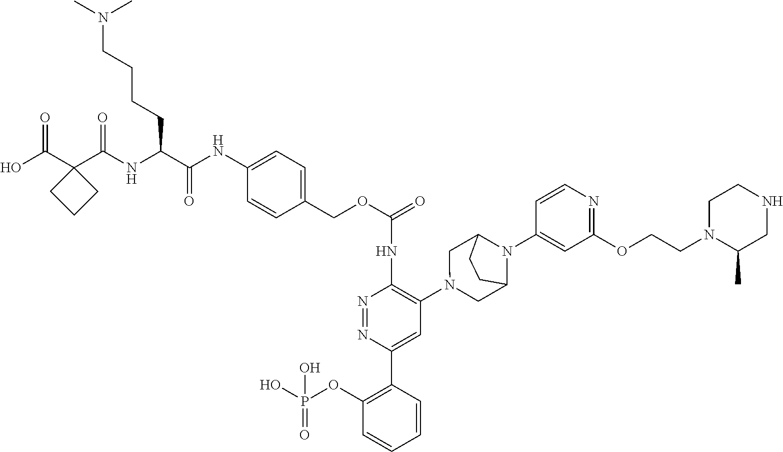 [SMILES: C[C@@H]1CNCCN1CCOc1cc(N2C3CCC2CN(c2cc(-c4ccccc4OP(=O)(O)O)nnc2NC(=O)OCc2ccc(NC(=O)[C@H](CCCCN(C)C)NC(=O)C4(C(=O)O)CCC4)cc2)C3)ccn1]

Under nitrogen, to a solution of tert-butyl (3R)-4-(2-((4-(3-(6-(2-((di-tert-butoxyphosphoryl)oxy)phenyl)-3-((((4-((S)-6-(dimethylamino)-2-(1-(ethoxycarbonyl)cyclobutane-1-carboxamido)hexanamido)benzyl)oxy)carbonyl)amino)pyridazin-4-yl)-3,8-diazabicyclo[3.2.1]octan-8-yl)pyridin-2-yl)oxy)ethyl)-3-methylpiperazine-1-carboxylate (35.0 mg, 0.0280 mmol) in THF (0.3 mL) was added a solution of LiOH·H₂O (3.5 mg, 0.0850 mmol) in H₂O (0.3 mL) at room temperature. The reaction was stirred at room temperature for 1 h. The resulting mixture was concentrated under vacuum. Then a solution of concentrated HCl (0.33 mL) in H₂O (0.67 mL) was added and stirred at room temperature for 10 min. The resulting mixture was basified to pH=8 with saturated NaHCO₃ solution at 0° C. and purified by pre-packed C18 column (solvent gradient: 0-100% MeOH in water (0.05% NH₄HCO₃)) to yield 13 mg of the title compound as a white solid. LCMS (ESI, m/z): [M+H]⁺=1029.

### Step 5: 1-(((2S)-6-(Dimethylamino)-1-((4-((((4-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)-6-(2-(phosphonooxy)phenyl)pyridazin-3-yl)carbamoyl)oxy)methyl)phenyl)amino)-1-oxohexan-2-yl)carbamoyl)cyclobutane-1-carboxylic acid (2,2,2-trifluoroacetic acid salt)

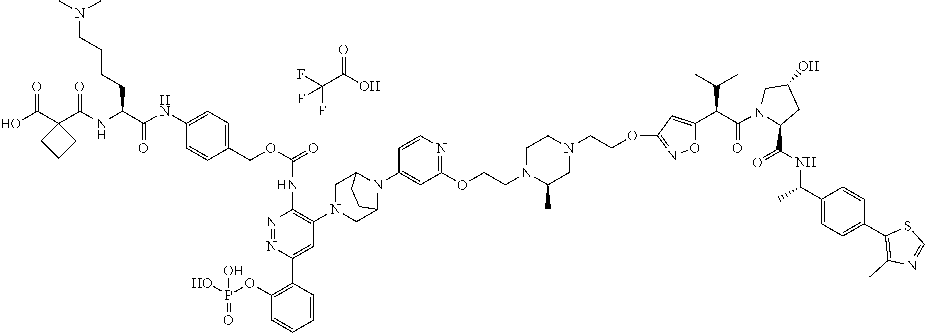 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7OP(=O)(O)O)nnc5NC(=O)OCc5ccc(NC(=O)[C@H](CCCCN(C)C)NC(=O)C7(C(=O)O)CCC7)cc5)C6)ccn4)[C@H](C)C3)no2)C(C)C)cc1 ; O=C(O)C(F)(F)F]

Under nitrogen, a solution of 1-(((2S)-6-(Dimethylamino)-1-((4-((((4-(8-(2-(2-((R)-2-methylpiperazin-1-yl)ethoxy)pyridin-4-yl)-3,8-diazabicyclo[3.2.1]octan-3-yl)-6-(2-(phosphonooxy)phenyl)pyridazin-3-yl)carbamoyl)oxy)methyl)phenyl)amino)-1-oxohexan-2-yl)carbamoyl)cyclobutane-1-carboxylic acid (18.0 mg, 0.0180 mmol), (2S,4R)-4-hydroxy-1-((R)-3-methyl-2-(3-(2-oxoethoxy)isoxazol-5-yl)butanoyl)-N—((S)-1-(4-(4-methylthiazol-5-yl)phenyl)ethyl)pyrrolidine-2-carboxamide (9.5 mg, 0.0180 mmol) and HOAc (2.1 mg, 0.0360 mmol) in DCM (0.75 mL) and MeOH (0.25 mL) was stirred at room temperature for 1 h. Then NaBH₃CN (1.7 mg, 0.0260 mmol) was added and stirred at room temperature for 0.5 h. The crude product was purified by Prep-HPLC with the following conditions: Column: Xselect CSH C18 OBD Column 30*150 mm 5 μm, n; Mobile Phase A: water (0.05% TFA), Mobile Phase B: ACN; Flow rate: 60 mL/min; Gradient: 11% B to 23% B in 12 min, 23% B; Wave Length: 254/220 nm; R_{T}(min): 12; to yield 6 mg of the title compound as a white solid. LCMS (ESI, m/z): [M+H]⁺=1553.

### Step 6: 4-((S)-6-(dimethylamino)-2-(1-((5-(2,5-dioxo-2,5-dihydro-1H-pyrrol-1-yl)pentyl)carbamoyl)cyclobutane-1-carboxamido)hexanamido)benzyl (4-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)-6-(2-(phosphonooxy)phenyl)pyridazin-3-yl)carbamate (2,2,2-trifluoroacetic acid salt)

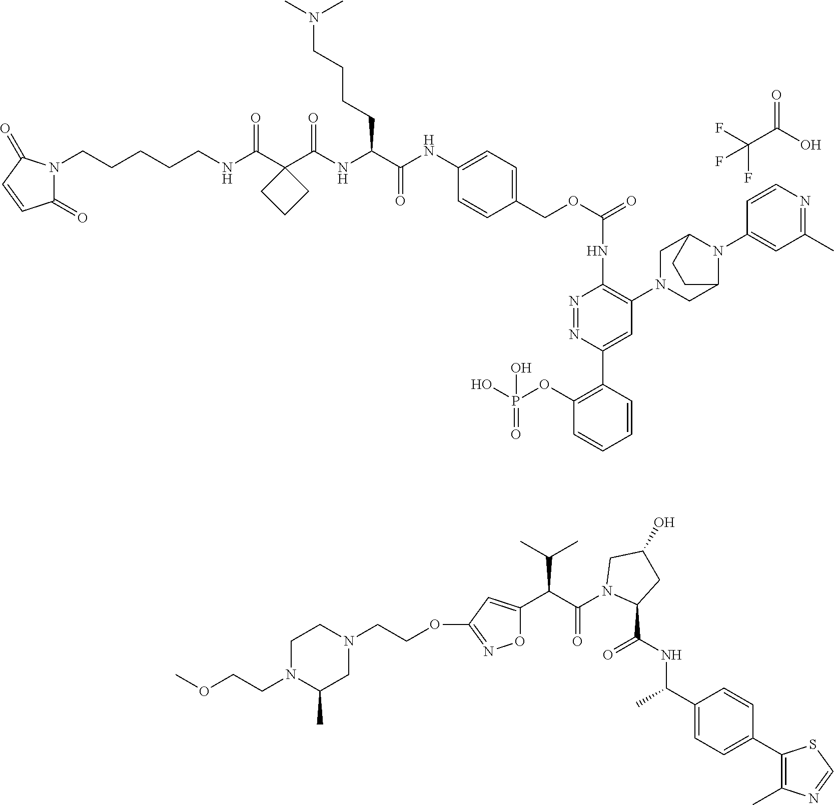

Under nitrogen, to a solution of 1-(((2S)-6-(dimethylamino)-1-((4-((((4-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)-6-(2-(phosphonooxy)phenyl)pyridazin-3-yl)carbamoyl)oxy)methyl)phenyl)amino)-1-oxohexan-2-yl)carbamoyl)cyclobutane-1-carboxylic acid (2,2,2-trifluoroacetic acid salt) (6.0 mg, 0.00400 mmol), 1-(5-aminopentyl)pyrrole-2,5-dione (2,2,2-trifluoroacetic acid salt) (0.9 mg, crude) and DIPEA (5.0 mg, 0.0400 mmol) in DMF (0.8 mL) was added HATU (1.9 mg, 0.00500 mmol) at room temperature. The reaction was stirred at room temperature for 10 min. The resulting mixture was purified by Prep-HPLC with the following conditions: Column: XBridge Prep C18 OBD Column, 30*100 mm, 5 μm; Mobile Phase A: Water (0.05% TFA), Mobile Phase B: ACN; Flow rate: 60 mL/min; Gradient: 15% B to 33% B in 11 min, 33% B; Wave Length: 254/220 nm; R_{T}(min): 11 to yield 2.7 mg of L1-PP-CIDE-1 as a white solid. LCMS (ESI, m/z): [M+H]⁺=1717. ¹H NMR (300 MHz, DMSO-d₆, ppm) δ 10.14 (d, J=11.3 Hz, 2H), 8.97 (s, 1H), 8.40 (d, J=7.7 Hz, 1H), 8.00-7.85 (m, 2H), 7.82-7.75 (s, 1H), 7.70-7.60 (m, 3H), 7.60-7.57 (m, 1H), 7.57-7.42 (m, 6H), 7.35-7.30 (m, 1H), 6.93 (s, 2H), 6.66 (d, J=6.6 Hz, 1H), 6.31 (s, 1H), 6.10 (s, 1H), 5.10 (s, 2H), 4.94-4.83 (m, 1H), 4.58 (s, 2H), 4.46 (s, 2H), 4.43-4.32 (m, 4H), 4.30-4.25 (m, 2H), 3.70-3.59 (m, 6H), 3.50-3.40 (m, 3H), 3.40-3.30 (m, 4H), 3.31-2.92 (m, 12H), 2.79-2.65 (m, 6H), 2.50-2.35 (m, 7H), 2.37-2.05 (m, 1H), 2.10-2.00 (m, 1H), 1.90 (s, 3H), 1.86-1.56 (m, 6H), 1.50-1.40 (m, 4H), 1.35 (d, J=7.0 Hz, 4H), 1.25-1.14 (m, 6H), 1.05-0.90 (m, 3H), 0.90-0.75 (m, 3H).

### Example 5

### Synthesis of L1-PP-CIDE-2

### L1-PP-CIDE-2: 4-((S)-6-(Dimethylamino)-2-(1-((5-(2,5-dioxo-2,5-dihydro-1H-pyrrol-1-yl)pentyl)carbamoyl)cyclobutane-1-carboxamido)hexanamido)benzyl (4-(8-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)-6-(2-(phosphonooxy)phenyl)pyridazin-3-yl)carbamate (2,2,2-trifluoroacetic acid salt)

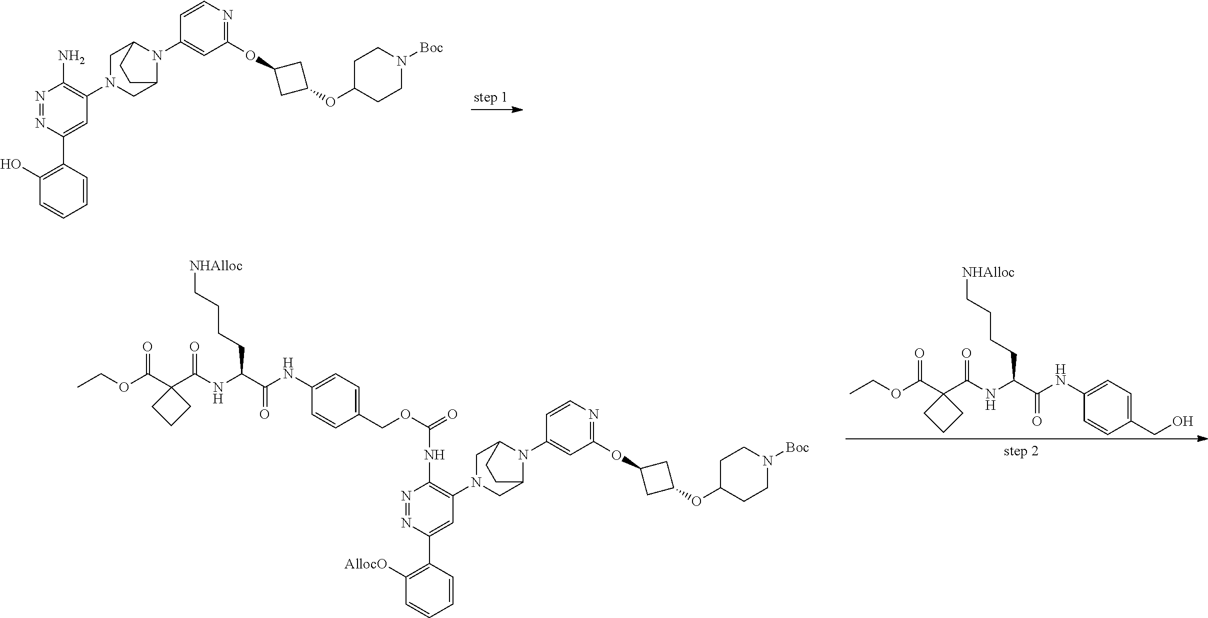

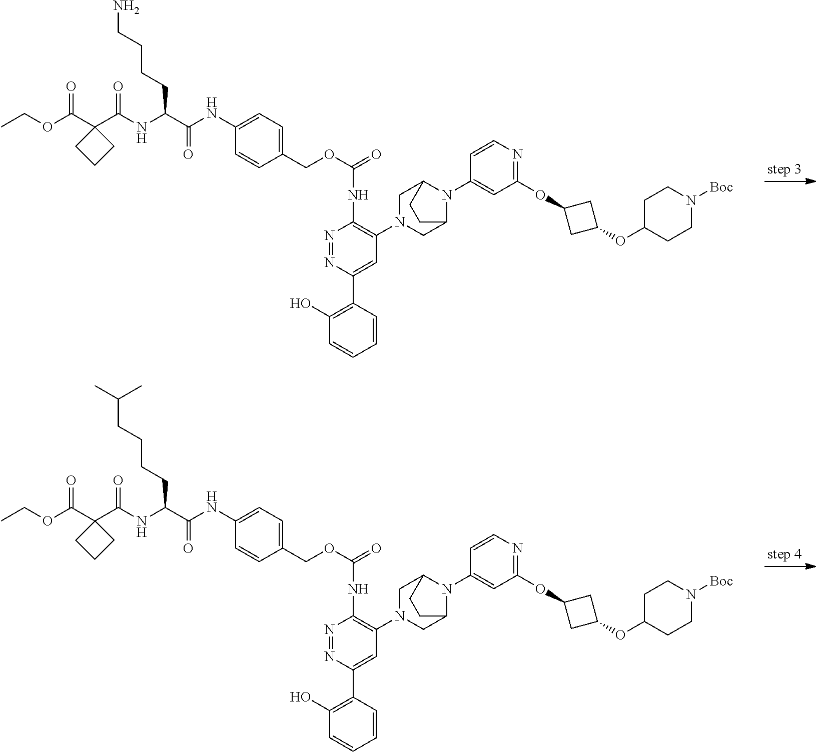 [SMILES: CCOC(=O)C1(C(=O)N[C@@H](CCCCN)C(=O)Nc2ccc(COC(=O)Nc3nnc(-c4ccccc4O)cc3N3CC4CCC(C3)N4c3ccnc(O[C@H]4C[C@H](OC5CCN(C(=O)OC(C)(C)C)CC5)C4)c3)cc2)CCC1 ; CCOC(=O)C1(C(=O)N[C@@H](CCCCC(C)C)C(=O)Nc2ccc(COC(=O)Nc3nnc(-c4ccccc4O)cc3N3CC4CCC(C3)N4c3ccnc(O[C@H]4C[C@H](OC5CCN(C(=O)OC(C)(C)C)CC5)C4)c3)cc2)CCC1]

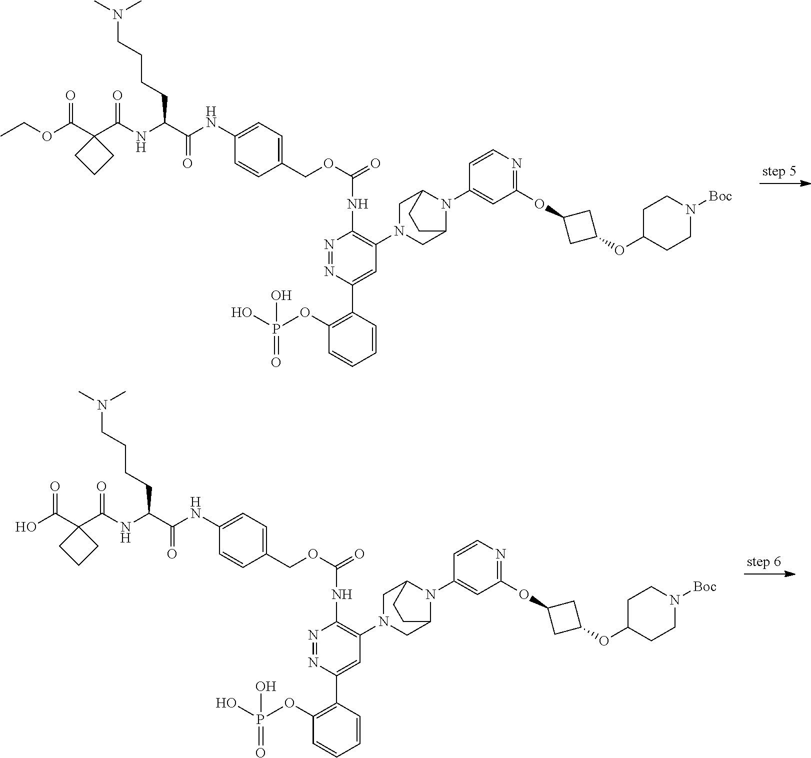 [SMILES: CCOC(=O)C1(C(=O)N[C@@H](CCCCN(C)C)C(=O)Nc2ccc(COC(=O)Nc3nnc(-c4ccccc4OP(=O)(O)O)cc3N3CC4CCC(C3)N4c3ccnc(O[C@H]4C[C@H](OC5CCN(C(=O)OC(C)(C)C)CC5)C4)c3)cc2)CCC1 ; CN(C)CCCC[C@H](NC(=O)C1(C(=O)O)CCC1)C(=O)Nc1ccc(COC(=O)Nc2nnc(-c3ccccc3OP(=O)(O)O)cc2N2CC3CCC(C2)N3c2ccnc(O[C@H]3C[C@H](OC4CCN(C(=O)OC(C)(C)C)CC4)C3)c2)cc1]

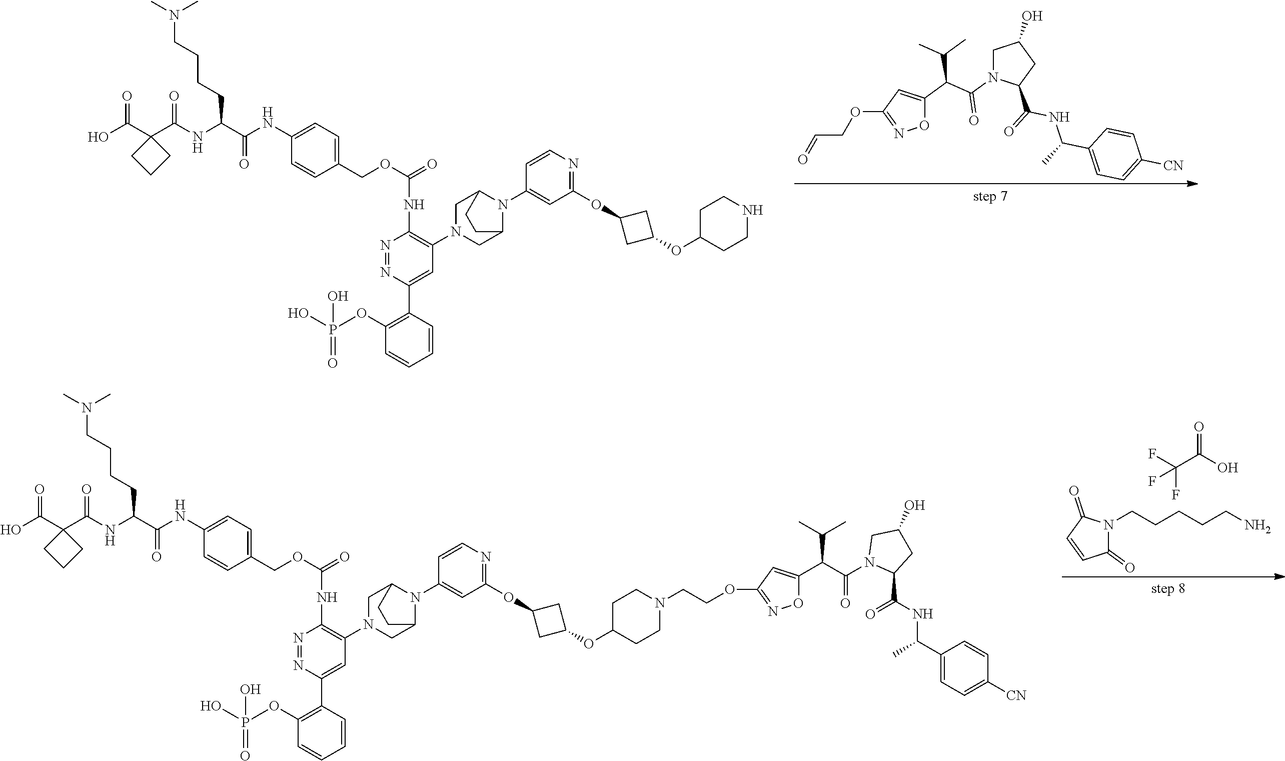

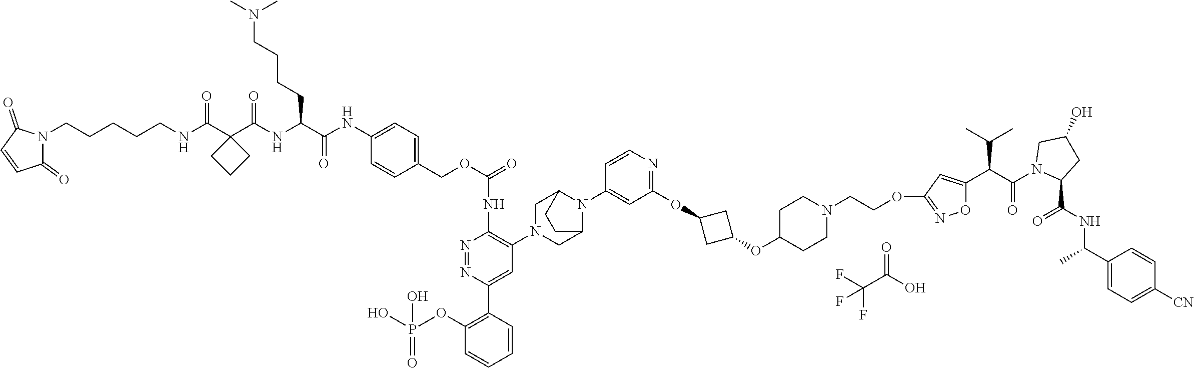 [SMILES: CC(C)[C@@H](C(=O)N1C[C@H](O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OP(=O)(O)O)nnc5NC(=O)OCc5ccc(NC(=O)[C@H](CCCCN(C)C)NC(=O)C7(C(=O)NCCCCCN8C(=O)C=CC8=O)CCC7)cc5)C6)ccn4)C3)CC2)no1 ; O=C(O)C(F)(F)F]

### Step 1: tert-Butyl 4-((1r,3r)-3-((4-(3-(6-(2-(((allyloxy)carbonyl)oxy)phenyl)-3-aminopyridazin-4-yl)-3,8-diazabicyclo[3.2.1]octan-8-yl)pyridin-2-yl)oxy)cyclobutoxy)piperidine-1-carboxylate

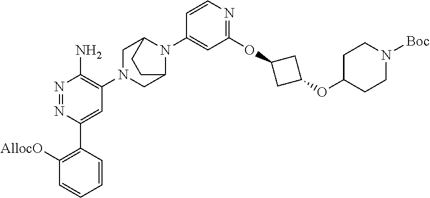

Under nitrogen, to a solution of tert-butyl 4-((1r,3r)-3-((4-(3-(3-amino-6-(2-hydroxyphenyl)pyridazin-4-yl)-3,8-diazabicyclo[3.2.1]octan-8-yl)pyridin-2-yl)oxy)cyclobutoxy)piperidine-1-carboxylate (535 mg, 0.832 mmol) and DIPEA (967 mg, 7.48 mmol) in DCM (6 mL) was added allyl carbonochloridate (350 mg, 2.91 mmol) at 0° C. The reaction was stirred at room temperature for 30 min. The crude was purified by flash chromatography on silica gel (solvent gradient: 0-10% MeOH in DCM) to yield 410 mg of the title compound as a white solid. LCMS (ESI) [M+H]=728.

### Step 2: tert-Butyl 4-((1r,3r)-3-((4-(3-(3-((((4-((S)-6-(((allyloxy)carbonyl)amino)-2-(1-(ethoxycarbonyl)cyclobutane-1-carboxamido)hexanamido)benzyl)oxy)carbonyl)amino)-6-(2-(((allyloxy)carbonyl)oxy)phenyl)pyridazin-4-yl)-3,8-diazabicyclo[3.2.1]octan-8-yl)pyridin-2-yl)oxy)cyclobutoxy)piperidine-1-carboxylate

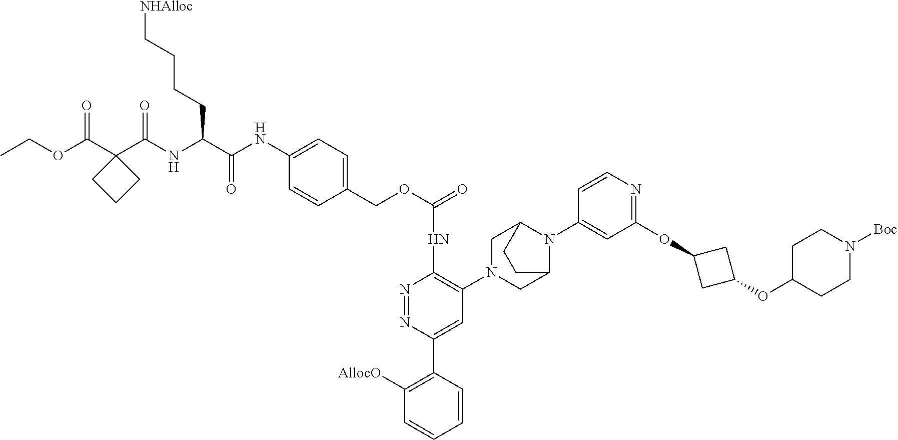

Under nitrogen, to a solution of tert-butyl 4-((1r,3r)-3-((4-(3-(6-(2-(((allyloxy)carbonyl)oxy)phenyl)-3-aminopyridazin-4-yl)-3,8-diazabicyclo[3.2.1]octan-8-yl)pyridin-2-yl)oxy)cyclobutoxy)piperidine-1-carboxylate (440 mg, 0.605 mmol) and ethyl (S)-1-((6-(((allyloxy)carbonyl)amino)-1-((4-(hydroxymethyl)phenyl)amino)-1-oxohexan-2-yl)carbamoyl)cyclobutane-1-carboxylate (921 mg, 1.81 mmol) and DIPEA (468 mg, 3.63 mmol) in THF (5 mL) was added triphosgene (89.8 mg, 0.302 mmol) at 0° C. The reaction was stirred at room temperature for 0.5 h. Then another triphosgene (36.0 mg, 0.121 mmol) was added at 0° C. and stirred at room temperature for 0.5 h. Aqueous NH₄Cl solution was added at 0° C. to quench the reaction. The solvent was concentrated under vacuum. The crude was purified by pre-packed C18 column (solvent gradient: 0-100% ACN in water (0.05% NH₄HCO₃)) to yield 280 mg of the title compound as a white solid. LCMS (ESI) [M+H]+=1244.

### Step 3: tert-Butyl 4-((1r,3r)-3-((4-(3-(3-((((4-((S)-6-amino-2-(1-(ethoxycarbonyl)cyclobutane-1-carboxamido)hexanamido)benzyl)oxy)carbonyl)amino)-6-(2-hydroxyphenyl)pyridazin-4-yl)-3,8-diazabicyclo[3.2.1]octan-8-yl)pyridin-2-yl)oxy)cyclobutoxy)piperidine-1-carboxylate

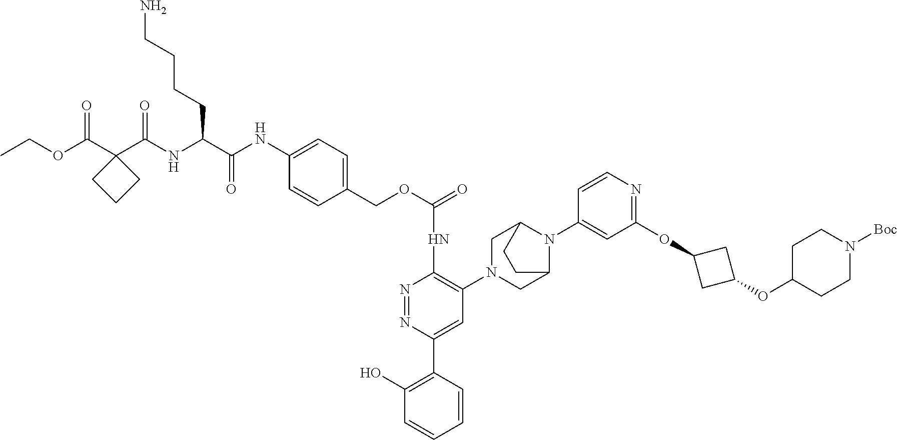 [SMILES: CCOC(=O)C1(C(=O)N[C@@H](CCCCN)C(=O)Nc2ccc(COC(=O)Nc3nnc(-c4ccccc4O)cc3N3CC4CCC(C3)N4c3ccnc(O[C@H]4C[C@H](OC5CCN(C(=O)OC(C)(C)C)CC5)C4)c3)cc2)CCC1]

Under nitrogen, to a solution of tert-butyl 4-((1r,3r)-3-((4-(3-(3-((((4-((S)-6-amino-2-(1-(ethoxycarbonyl)cyclobutane-1-carboxamido)hexanamido)benzyl)oxy)carbonyl)amino)-6-(2-hydroxyphenyl)pyridazin-4-yl)-3,8-diazabicyclo[3.2.1]octan-8-yl)pyridin-2-yl)oxy)cyclobutoxy)piperidine-1-carboxylate (260 mg, 0.209 mmol) and pyrrolidine (148 mg, 2.09 mmol) in DCM (5 mL) was added Pd(PPh₃)₄ (24.1 mg, 0.0208 mmol) at room temperature. The reaction was stirred at room temperature for 1 h. Water was added and DCM was used to extract for three times. Organic layers were combined, dried over anhydrous Na₂SO₄ and concentrated under vacuum to to yield 310 mg (crude) of the title compound which was used in the next step directly. LCMS (ESI) [M+H]⁺=1076.

### Step 4: tert-Butyl 4-((1r,3r)-3-((4-(3-(3-((((4-((S)-6-(dimethylamino)-2-(1-(ethoxycarbonyl)cyclobutane-1-carboxamido)hexanamido)benzyl)oxy)carbonyl)amino)-6-(2-hydroxyphenyl)pyridazin-4-yl)-3,8-diazabicyclo[3.2.1]octan-8-yl)pyridin-2-yl)oxy)cyclobutoxy)piperidine-1-carboxylate

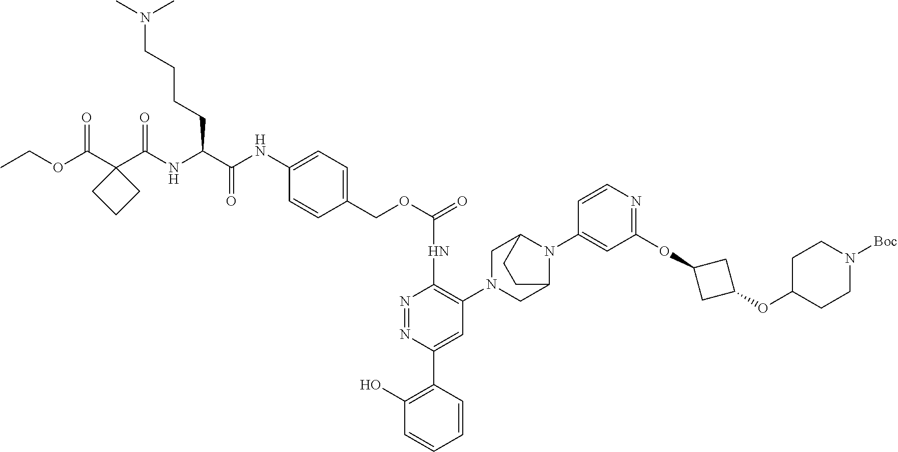 [SMILES: CCOC(=O)C1(C(=O)N[C@@H](CCCCN(C)C)C(=O)Nc2ccc(COC(=O)Nc3nnc(-c4ccccc4O)cc3N3CC4CCC(C3)N4c3ccnc(O[C@H]4C[C@H](OC5CCN(C(=O)OC(C)(C)C)CC5)C4)c3)cc2)CCC1]

A solution of tert-butyl 4-((1r,3r)-3-((4-(3-(3-((((4-((S)-6-amino-2-(1-(ethoxycarbonyl)cyclobutane-1-carboxamido)hexanamido)benzyl)oxy)carbonyl)amino)-6-(2-hydroxyphenyl)pyridazin-4-yl)-3,8-diazabicyclo[3.2.1]octan-8-yl)pyridin-2-yl)oxy)cyclobutoxy)piperidine-1-carboxylate (310 mg, crude) and aqueous CH₂O (37%) (259 mg, 3.19 mmol) in MeOH (3 mL) was stirred at room temperature for 1 h. Then NaBH₃CN (75.9 mg, 1.21 mmol) was added and stirred at room temperature for 1 h. The crude was purified by pre-packed C18 column (solvent gradient: 0-100% MeOH in water (0.05% NH₄HCO₃)) to yield 120 mg of the title compound as a white solid. LCMS (ESI) [M+H]+=1104.

### Step 5: 1-(((2S)-1-((4-((((4-(8-(2-((1r,3r)-3-((1-(tert-Butoxycarbonyl)piperidin-4-yl)oxy)cyclobutoxy)pyridin-4-yl)-3,8-diazabicyclo[3.2.1]octan-3-yl)-6-(2-(phosphonooxy)phenyl)pyridazin-3-yl)carbamoyl)oxy)methyl)phenyl)amino)-6-(dimethylamino)-1-oxohexan-2-yl)carbamoyl)cyclobutane-1-carboxylic acid

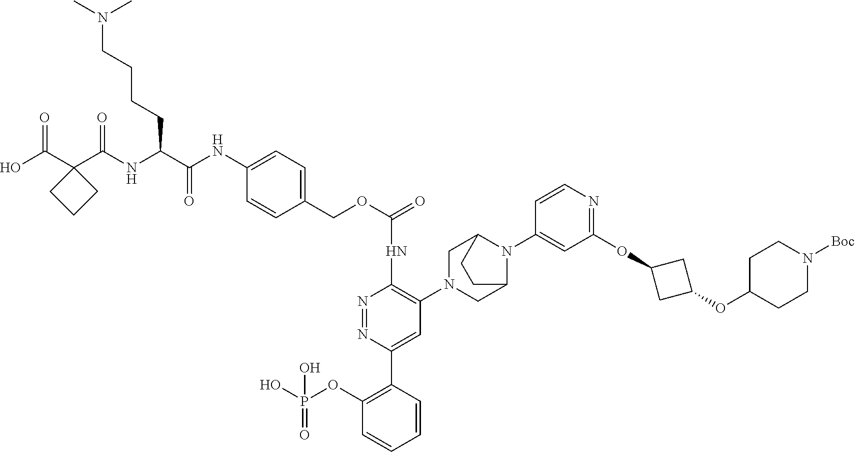 [SMILES: CN(C)CCCC[C@H](NC(=O)C1(C(=O)O)CCC1)C(=O)Nc1ccc(COC(=O)Nc2nnc(-c3ccccc3OP(=O)(O)O)cc2N2CC3CCC(C2)N3c2ccnc(O[C@H]3C[C@H](OC4CCN(C(=O)OC(C)(C)C)CC4)C3)c2)cc1]

Under nitrogen, to a solution of tert-butyl 4-((1r,3r)-3-((4-(3-(3-((((4-((S)-6-(dimethylamino)-2-(1-(ethoxycarbonyl)cyclobutane-1-carboxamido)hexanamido)benzyl)oxy)carbonyl)amino)-6-(2-hydroxyphenyl)pyridazin-4-yl)-3,8-diazabicyclo[3.2.1]octan-8-yl)pyridin-2-yl)oxy)cyclobutoxy)piperidine-1-carboxylate (120 mg, 0.108 mmol) in THF (3 mL) was added a solution of TMSOK in THF (1M, 2 mL) at −60° C. Then POCl₃ (165 mg, 1.08 mmol) was added at −60° C. The reaction was stirred at −60° C. for 20 min. Then NaOH (80.0 mg, 2.00 mmol) in H₂O (1.6 mL) was added at −60° C. and stirred at room temperature for 4 h. The crude was purified by pre-packed C18 column (solvent gradient: 0-100% MeOH in water (0.05% NH₄HCO₃)) to yield 65.0 mg of the title compound as a white solid. LCMS (ESI) [M+H]⁺=1156.

### Step 6: 1-(((2S)-6-(Dimethylamino)-1-oxo-1-((4-((((6-(2-(phosphonooxy)phenyl)-4-(8-(2-((1r,3r)-3-(piperidin-4-yloxy)cyclobutoxy)pyridin-4-yl)-3,8-diazabicyclo[3.2.1]octan-3-yl)pyridazin-3-yl)carbamoyl)oxy)methyl)phenyl)amino)hexan-2-yl)carbamoyl)cyclobutane-1-carboxylic acid

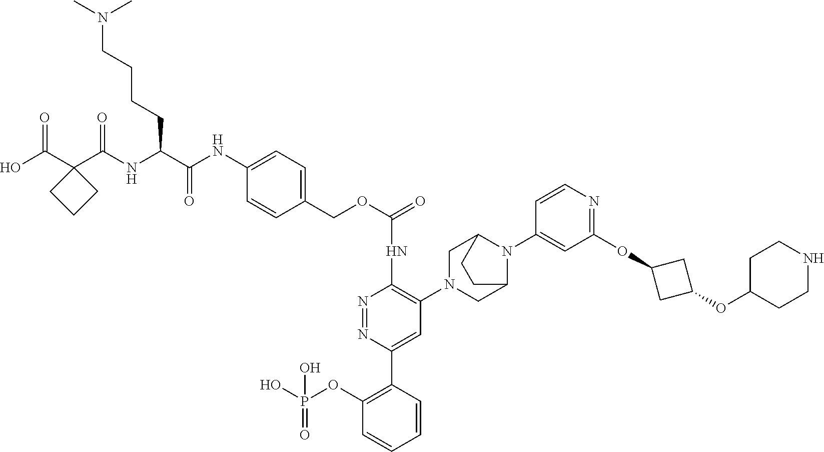 [SMILES: CN(C)CCCC[C@H](NC(=O)C1(C(=O)O)CCC1)C(=O)Nc1ccc(COC(=O)Nc2nnc(-c3ccccc3OP(=O)(O)O)cc2N2CC3CCC(C2)N3c2ccnc(O[C@H]3C[C@H](OC4CCNCC4)C3)c2)cc1]

A solution of 1-(((2S)-1-((4-((((4-(8-(2-((1r,3r)-3-((1-(tert-butoxycarbonyl)piperidin-4-yl)oxy)cyclobutoxy)pyridin-4-yl)-3,8-diazabicyclo[3.2.1]octan-3-yl)-6-(2-(phosphonooxy)phenyl)pyridazin-3-yl)carbamoyl)oxy)methyl)phenyl)amino)-6-(dimethylamino)-1-oxohexan-2-yl)carbamoyl)cyclobutane-1-carboxylic acid (65.0 mg, 0.056 mmo) in a solution of 5% TFA in HFIP (6.1 mL) was stirred at room temperature for 15 min. Solvent was evaporated and the crude was purified by pre-packed C18 column (0-100% ACN in water (0.05% NH₄HCO₃)) to yield 58.0 mg of the title compound as a yellow oil. LCMS (ESI) [M+H]⁺=1055.

### Step 7: 1-(((2S)-1-((4-((((4-(8-(2-((1R,3r)-3-((1-(2-((5-((R)-1-((2S,4R)-2-(((S)-1-(4-Cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)-6-(2-(phosphonooxy)phenyl)pyridazin-3-yl)carbamoyl)oxy)methyl)phenyl)amino)-6-(dimethylamino)-1-oxohexan-2-yl)carbamoyl)cyclobutane-1-carboxylic acid

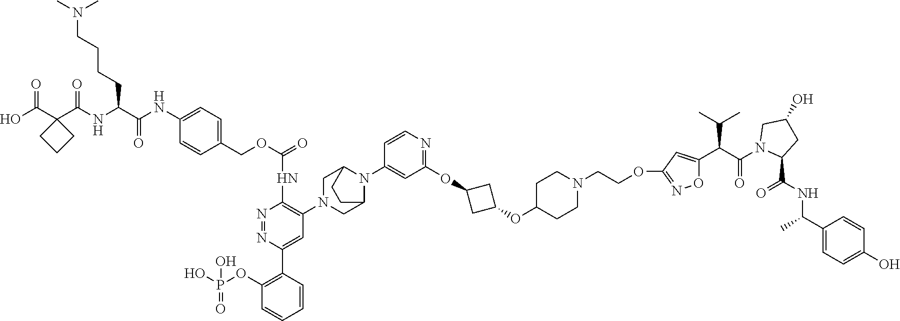 [SMILES: CC(C)[C@@H](C(=O)N1C[C@H](O)C[C@H]1C(=O)N[C@@H](C)c1ccc(O)cc1)c1cc(OCCN2CCC(O[C@H]3C[C@H](Oc4cc(N5C6CCC5CN(c5cc(-c7ccccc7OP(=O)(O)O)nnc5NC(=O)OCc5ccc(NC(=O)[C@H](CCCCN(C)C)NC(=O)C7(C(=O)O)CCC7)cc5)C6)ccn4)C3)CC2)no1]

A solution of 1-(((2S)-6-(dimethylamino)-1-oxo-1-((4-((((6-(2-(phosphonooxy)phenyl)-4-(8-(2-((1r,3r)-3-(piperidin-4-yloxy)cyclobutoxy)pyridin-4-yl)-3,8-diazabicyclo[3.2.1]octan-3-yl)pyridazin-3-yl)carbamoyl)oxy)methyl)phenyl)amino)hexan-2-yl)carbamoyl)cyclobutane-1-carboxylic acid (58.0 mg, 0.055 mmol), (2S,4R)—N—((S)-1-(4-cyanophenyl)ethyl)-4-hydroxy-1-((R)-3-methyl-2-(3-(2-oxoethoxy)isoxazol-5-yl)butanoyl)pyrrolidine-2-carboxamide (73.5 mg, 0.157 mmol) and HOAc (6.6 mg, 0.11 mmol) in MeOH (1 mL) and DCM (3 mL) was stirred at room temperature for 1 h. Then NaBH₃CN (5.1 mg, 0.083 mmol) was added and stirred at room temperature for 0.5 h. The crude was purified by pre-packed C18 column (solvent gradient: 0-100% MeOH in water (0.05% NH₄HCO₃)) to yield 44.0 mg of the title compound as a white solid. LCMS (ESI) [M+H]=1508.

### Step 8: 4-((S)-6-(Dimethylamino)-2-(1-((5-(2,5-dioxo-2,5-dihydro-1H-pyrrol-1-yl)pentyl)carbamoyl)cyclobutane-1-carboxamido)hexanamido)benzyl (4-(8-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)-6-(2-(phosphonooxy)phenyl)pyridazin-3-yl)carbamate (2,2,2-trifluoroacetic acid salt)

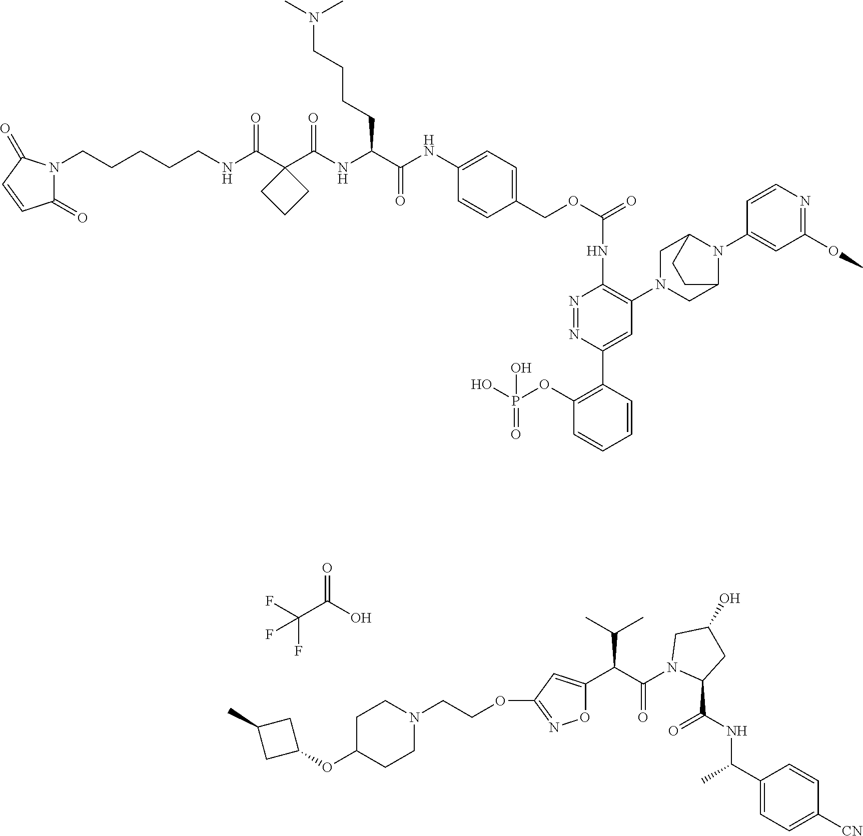

A solution of 1-(((2S)-1-((4-((((4-(8-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)-6-(2-(phosphonooxy)phenyl)pyridazin-3-yl)carbamoyl)oxy)methyl)phenyl)amino)-6-(dimethylamino)-1-oxohexan-2-yl)carbamoyl)cyclobutane-1-carboxylic acid (44.0 mg, 0.0292 mmol) and 1-(5-aminopentyl)-1H-pyrrole-2,5-dione (2,2,2-trifluoroacetic acid) (19.9 mg, crude), HATU (14.4 mg, 0.0378 mmol) and DIPEA (37.7 mg, 0.290 mmol) in DMF (0.8 mL) was stirred at room temperature for 10 min. The crude was purified by Prep HPLC with the following conditions: Column: XBridge Shield RP18 OBD Column, 30*150 mm, 5 μm; Mobile Phase A: Water (0.05% TFA), Mobile Phase B: ACN; Flow rate: 60 mL/min; Gradient: 15% B to 25% B in 20 min, 25% B; Wavelength: 254/220 nm; R_{T} (min): 20 to yield 13.6 mg of L1-PP-CIDE-2 as a white solid. LCMS (ESI) [M+H]⁺=1672. ¹H NMR (400 MHz, DMSO-d₆, ppm) δ 10.19 (s, 2H), 9.45 (s, 1H), 8.50 (d, J=7.3 Hz, 1H), 7.95-7.85 (m, 3H), 7.83-7.76 (m, 3H), 7.72-7.64 (m, 2H), 7.62-7.49 (m, 2H), 7.48-7.30 (m, 5H), 7.24-6.98 (m, 3H), 6.72 (s, 1H), 6.20 (s, 1H), 6.15 (s, 1H), 5.24-5.09 (m, 3H), 4.91 (q, J=7.2 Hz, 1H), 4.69 (s, 2H), 4.53 (d, J=5.1 Hz, 2H), 4.47-4.24 (m, 5H), 3.70-3.60 (m, 7H), 3.45-3.40 (m, 2H), 3.38-3.34 (m, 2H), 3.19-3.06 (m, 6H), 3.05-2.98 (m, 3H), 2.75 (d, J=3.8 Hz, 6H), 2.46-2.35 (m, 6H), 2.30-2.20 (m, 1H), 2.09-1.99 (m, 2H), 1.99-1.85 (m, 5H), 1.84-1.56 (m, 8H), 1.54-1.41 (m, 5H), 1.40-1.27 (m, 5H), 1.26-1.13 (m, 3H), 1.01-0.93 (m, 3H), 0.87-0.72 (m, 3H).

### Example 6

### Synthesis of L1Hy-PE-CIDE-4

### L1Hy-PE-CIDE-4: (3R,5S)-1-((2R)-2-(3-(2-((3R)-4-(2-((4-(3-(3-Amino-6-(2-((4-((S)-6-(dimethylamino)-2-(1-((5-((2-(2,5-dioxo-2,5-dihydro-1H-pyrrol-1-yl)ethyl)(methyl)amino)pentyl)carbamoyl)cyclobutane-1-carboxamido)hexanamido)benzyl)oxy)phenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoyl)-5-(((S)-1-(4-(4-methylthiazol-5-yl)phenyl)ethyl)carbamoyl)pyrrolidin-3-yl dihydrogen phosphate (2,2,2-trifluoroacetic acid salt)

### Step 1: di-tert-butyl ((3R,5S)-1-((R)-2-(3-(2,2-diethoxyethoxy)isoxazol-5-yl)-3-methylbutanoyl)-5-(((S)-1-(4-(4-methylthiazol-5-yl)phenyl)ethyl)carbamoyl)pyrrolidin-3-yl) phosphate

Under nitrogen, to a solution of (2S,4R)-1-((R)-2-(3-(2,2-diethoxyethoxy)isoxazol-5-yl)-3-methylbutanoyl)-4-hydroxy-N—((S)-1-(4-(4-methylthiazol-5-yl)phenyl)ethyl)pyrrolidine-2-carboxamide (500 mg, 0.81 mmol) and 1H-tetrazole (5.4 mL, 0.45 M in ACN) in THF (5 mL) was added di-tert-butyl diisopropylphosphoramidite (451 mg, 1.63 mmol) dropwise at 0° C. The reaction was stirred at room temperature overnight. Then tBuOOH (0.4 mL, 5 M in decane) was added at −30° C. and stirred at −30° C. for 30 min. Then the mixture was stirred at room temperature for 4 hours. The reaction was quenched with aqueous NaHSO3 and extracted with EtOAc. The combined organic layer was washed with water and brine. The organic layer was dried over anhydrous sodium sulfate and concentrated under vacuum. The residue was purified by pre-packed C18 column (solvent gradient: 0-70% ACN in water (0.05% NH4HCO3)) to yield 340 mg (51%) of the title compound as a yellow oil. LC-MS: (ESI, m/z): [M+H]⁺=807.

### Step 2: (3R,5S)-1-((R)-3-methyl-2-(3-(2-oxoethoxy)isoxazol-5-yl)butanoyl)-5-(((S)-1-(4-(4-methylthiazol-5-yl)phenyl)ethyl)carbamoyl)pyrrolidin-3-yl dihydrogen phosphate

Under nitrogen, a solution of di-tert-butyl ((3R,5S)-1-((R)-2-(3-(2,2-diethoxyethoxy)isoxazol-5-yl)-3-methylbutanoyl)-5-(((S)-1-(4-(4-methylthiazol-5-yl)phenyl)ethyl)carbamoyl)pyrrolidin-3-yl) phosphate (150 mg, 0.185 mmol) in HCOOH (0.5 mL) and water (0.5 mL) was stirred at 60° C. for 1 h. The solution was concentrated under vacuum to afford 121 mg (crude) of the title compound as a solid. LC-MS: (ESI, m/z): [M+H]⁺=695.

### Step 3: 1-(((2S)-1-((4-((2-(6-Amino-5-(8-(2-(2-((R)-2-methyl-4-(2-((5-((R)-3-methyl-1-((2S,4R)-2-(((S)-1-(4-(4-methylthiazol-5-yl)phenyl)ethyl)carbamoyl)-4-(phosphonooxy)pyrrolidin-1-yl)-1-oxobutan-2-yl)isoxazol-3-yl)oxy)ethyl)piperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid

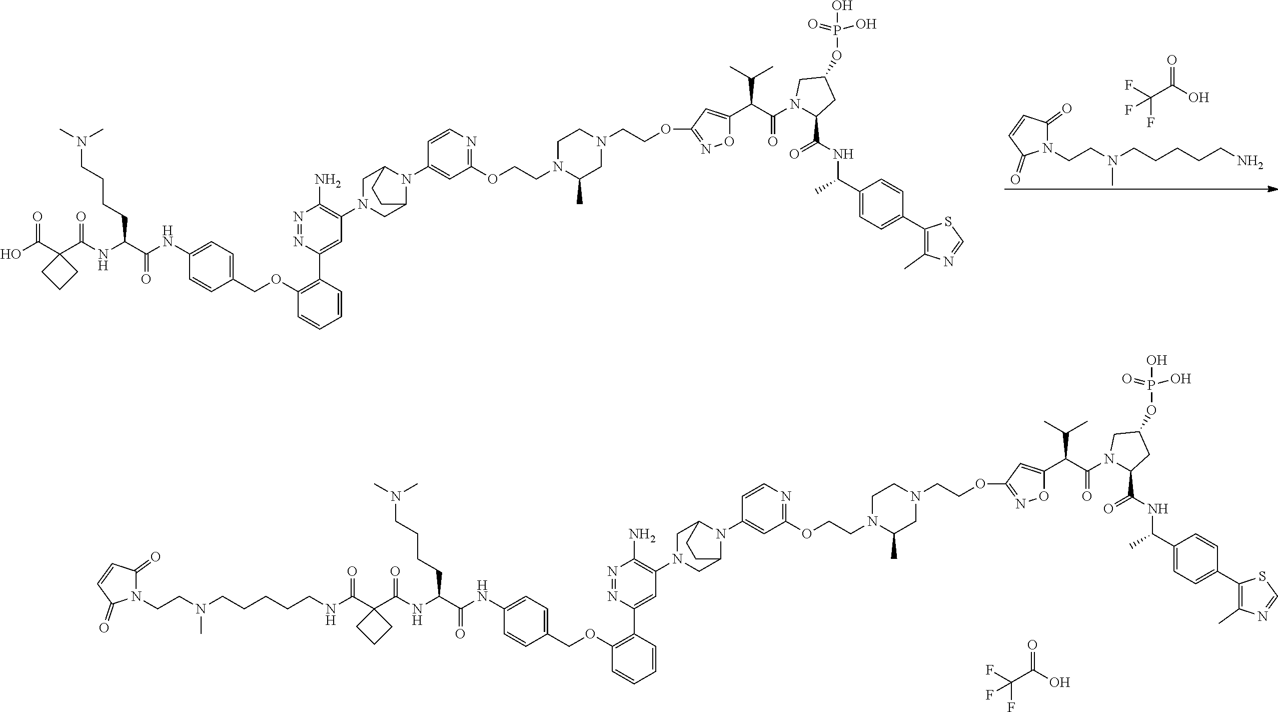

### Step 1: (3R,5S)-1-((2R)-2-(3-(2-((3R)-4-(2-((4-(3-(3-Amino-6-(2-((4-((S)-6-(dimethylamino)-2-(1-((5-((2-(2,5-dioxo-2,5-dihydro-1H-pyrrol-1-yl)ethyl)(methyl)amino)pentyl)carbamoyl)cyclobutane-1-carboxamido)hexanamido)benzyl)oxy)phenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoyl)-5-(((S)-1-(4-(4-methylthiazol-5-yl)phenyl)ethyl)carbamoyl)pyrrolidin-3-yl dihydrogen phosphate (2,2,2-trifluoroacetic acid salt)

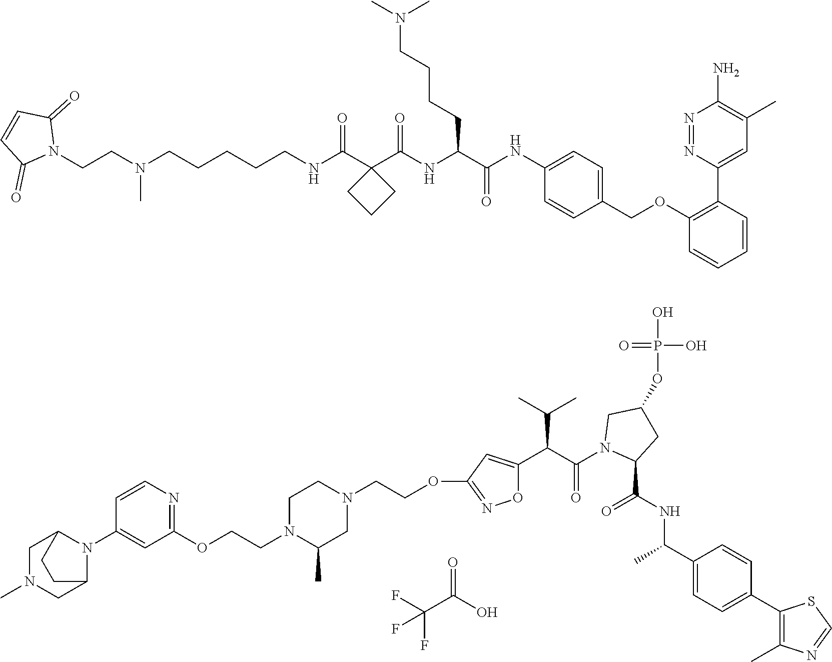

A solution of 1-(((2S)-1-((4-((2-(6-amino-5-(8-(2-(2-((R)-2-methyl-4-(2-((5-((R)-3-methyl-1-((2S,4R)-2-(((S)-1-(4-(4-methylthiazol-5-yl)phenyl)ethyl)carbamoyl)-4-(phosphonooxy)pyrrolidin-1-yl)-1-oxobutan-2-yl)isoxazol-3-yl)oxy)ethyl)piperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (40.0 mg, 0.0265 mmol), 1-(2-((5-aminopentyl)(methyl)amino)ethyl)-1H-pyrrole-2,5-dione (2,2,2-trifluoroacetic acid salt) (10.7 mg, crude), HATU (12.1 mg, 0.0318 mmol) and DIPEA (34.2 mg, 0.265 mmol) in DMF (0.8 mL) was stirred at room temperature for 10 min. The product was purified by Prep-HPLC with the following conditions: Column: XSelect CSH Fluoro Phenyl, 30*150 mm, 5 μm; Mobile Phase A: Water (0.05% TFA), Mobile Phase B: ACN; Flow rate: 60 mL/min; Gradient: 6% B to 36% B in 7 min; Wavelength: 254 nm; R_{T}(min): 6.5 to afford 7.0 mg of L1Hy-PE-CIDE-4 as a white solid. LCMS (ESI) [M+H]⁺=1730. ¹H NMR (400 MHz, DMSO-d₆, ppm) δ 10.23 (s, 1H), 9.00 (d, J=1.8 Hz, 1H), 8.48 (d, J=7.7 Hz, 1H), 7.93-7.83 (m, 3H), 7.67 (d, J=8.3 Hz, 2H), 7.59 (d, J=7.9 Hz, 1H), 7.56 (s, 1H), 7.49-7.31 (m, 7H), 7.18-7.07 (m, 3H), 6.90 (br, 1H), 6.64 (s, 1H), 6.26 (s, 1H), 6.11 (s, 1H), 5.10 (s, 2H), 4.92 (t, J=7.1 Hz, 1H), 4.82-4.70 (m, 2H), 4.48 (s, 5H), 4.41-4.33 (m, 3H), 3.80-3.65 (m, 6H), 3.15-2.96 (m, 18H), 2.79 (s, 4H), 2.73 (s, 6H), 2.50-2.35 (m, 9H), 2.05-1.85 (m, 6H), 1.78-1.65 (m, 3H), 1.65-1.40 (m, 8H), 1.39 (d, J=6.9 Hz, 3H), 1.35-1.18 (m, 7H), 0.98 (d, J=6.5 Hz, 3H), 0.90-0.75 (m, 3H).

### Example 7

### Synthesis of L1-PE-CIDE-5

### L1-PE-CIDE-5: (3R,5S)-1-((2R)-2-(3-(2-((3R)-4-(2-((4-(3-(3-Amino-6-(2-((4-((S)-6-(dimethylamino)-2-(1-((5-(2,5-dioxo-2,5-dihydro-1H-pyrrol-1-yl)pentyl)carbamoyl)cyclobutane-1-carboxamido)hexanamido)benzyl)oxy)phenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoyl)-5-(((S)-1-(4-(4-methylthiazol-5-yl)phenyl)ethyl)carbamoyl)pyrrolidin-3-yl dihydrogen phosphate (2,2,2-trifluoroacetic acid salt)

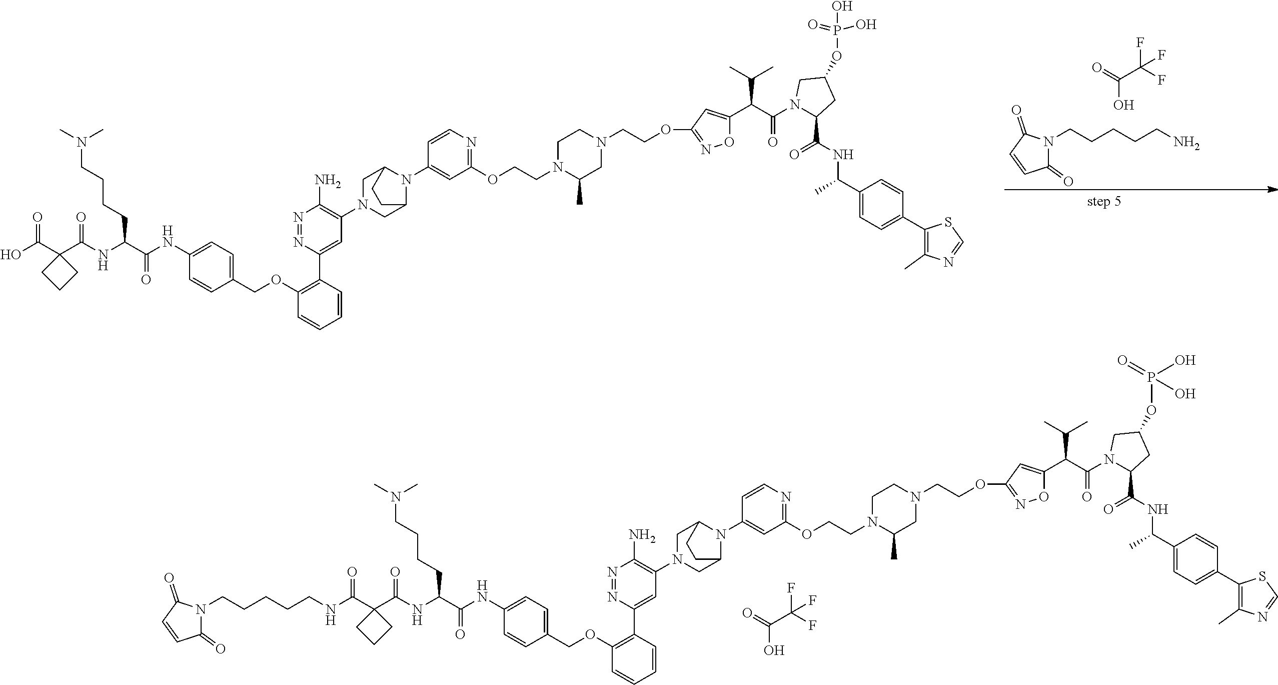

Step 1: (3R,5S)-1-((2R)-2-(3-(2-((3R)-4-(2-((4-(3-(3-Amino-6-(2-((4-((S)-6-(dimethylamino)-2-(1-((5-(2,5-dioxo-2,5-dihydro-1H-pyrrol-1-yl)pentyl)carbamoyl)cyclobutane-1-carboxamido)hexanamido)benzyl)oxy)phenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoyl)-5-(((S)-1-(4-(4-methylthiazol-5-yl)phenyl)ethyl)carbamoyl)pyrrolidin-3-yl dihydrogen phosphate (2,2,2-trifluoroacetic acid salt)

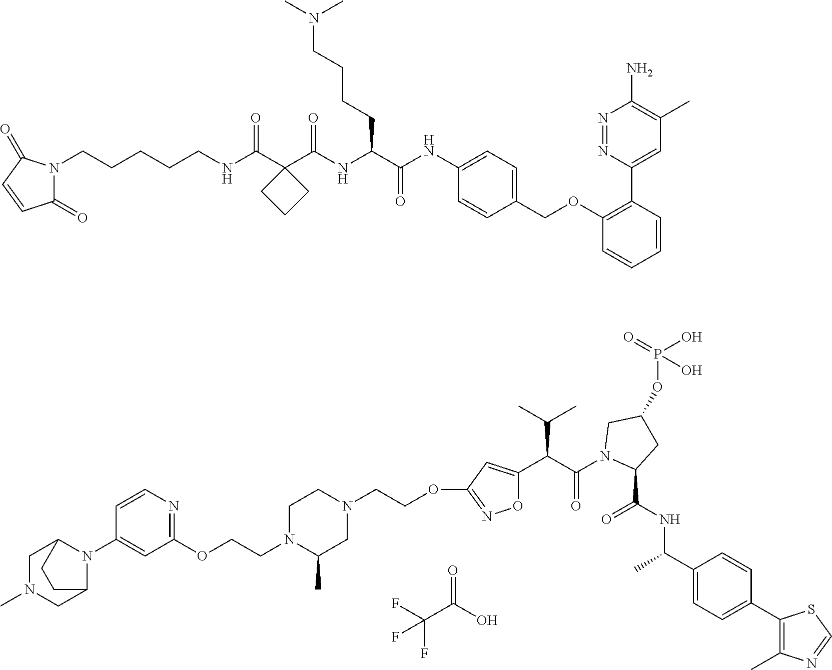

A solution of 1-(((2S)-1-((4-((2-(6-amino-5-(8-(2-(2-((R)-2-methyl-4-(2-((5-((R)-3-methyl-1-((2S,4R)-2-(((S)-1-(4-(4-methylthiazol-5-yl)phenyl)ethyl)carbamoyl)-4-(phosphonooxy)pyrrolidin-1-yl)-1-oxobutan-2-yl)isoxazol-3-yl)oxy)ethyl)piperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (180 mg, 0.119 mmol, synthesis described in L1-PE-CIDE-4) 1-(5-aminopentyl)-1H-pyrrole-2,5-dione (2,2,2-trifluoroacetic acid salt) (45.0 mg, crude), HATU (60.6 mg, 0.159 mmol) and DIPEA (158 mg, 1.22 mmol) in DMF (3 mL) was stirred at room temperature for 5 min. The reaction solution was purified by Prep-HPLC with the following conditions: Column: XSelect CSH Prep C18 OBD Column, 19*250 mm, 5 μm; Mobile Phase A: Water (0.05% TFA), Mobile Phase B: MeOH; Flow rate: 25 mL/min; Gradient: 46% B to 57% B in 11 min, 57% B; Wavelength: 254/220 nm; R_{T}(min): 8.75 to yield 66.6 mg of L1-PE-CIDE-5 as a while solid. LCMS (ESI) [M+H]⁺=1673. ¹H NMR (300 MHz, DMSO-d₆, ppm) δ 10.21 (s, 1H), 9.50 (s, 1H), 9.00 (d, J=1.7 Hz, 1H), 8.49 (d, J=7.6 Hz, 1H), 8.01-7.84 (m, 3H), 7.67 (d, J=8.3 Hz, 2H), 7.59 (dd, J=8.2, 6.1 Hz, 2H), 7.52-7.25 (m, 7H), 7.21-7.08 (m, 2H), 6.99 (s, 2H), 6.73 (d, J=6.7 Hz, 1H), 6.37 (s, 1H), 6.12 (s, 1H), 5.10 (s, 2H), 4.92 (t, J=7.3 Hz, 1H), 4.85-4.73 (m, 2H), 4.60 (s, 3H), 4.51-4.33 (m, 6H), 3.85-3.70 (m, 7H), 3.55-3.45 (m, 2H), 3.40-3.30 (m, 4H), 3.25-2.95 (m, 9H), 2.90-2.70 (m, 7H), 2.47-2.30 (m, 9H), 2.10-1.85 (m, 5H), 1.85-1.54 (m, 6H), 1.53-1.29 (m, 8H), 1.27-1.10 (m, 5H), 0.98 (d, J=6.5 Hz, 3H), 0.90-0.73 (m, 3H).

### Example 8

### Synthesis of L1Hy-PE-PCIDE-6

### (3R,5S)-1-((2R)-2-(3-(2-((3R)-4-(2-((4-(3-(3-amino-6-(2-((4-((S)-6-(dimethylamino)-2-(1-((2-((2-(2,5-dioxo-2,5-dihydro-1H-pyrrol-1-yl)ethyl)(methyl)amino)ethyl)carbamoyl)cyclobutane-1-carboxamido)hexanamido)benzyl)oxy)phenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoyl)-5-(((S)-1-(4-(4-methylthiazol-5-yl)phenyl)ethyl)carbamoyl)pyrrolidin-3-yl dihydrogen phosphate (2,2,2-trifluoroacetic acid salt)

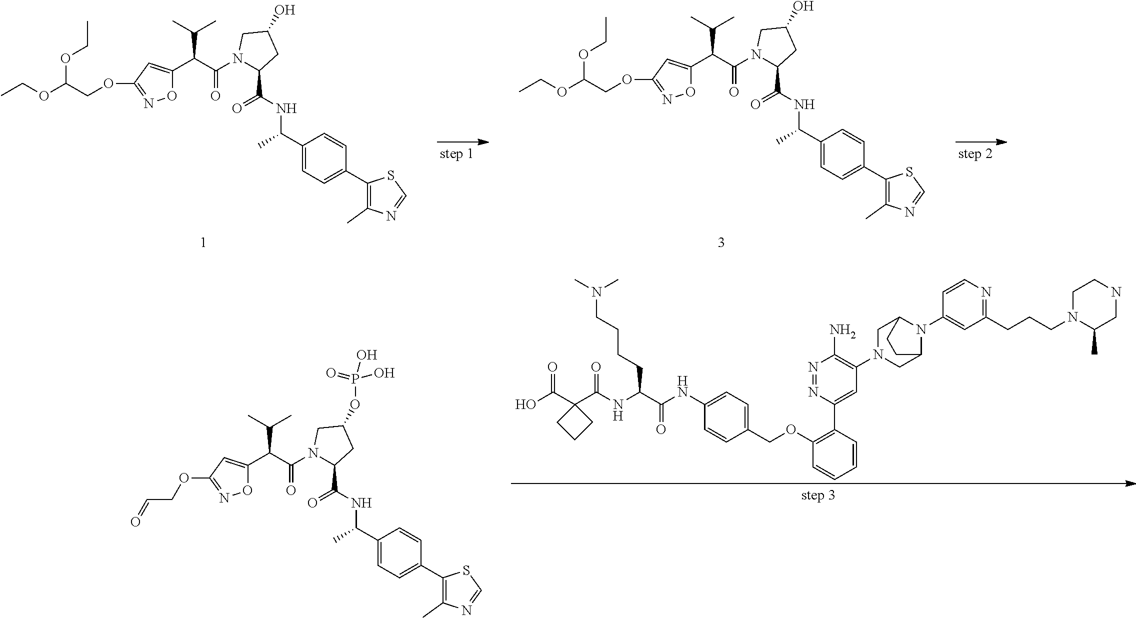 [SMILES: CCOC(COc1cc([C@H](C(=O)N2C[C@H](O)C[C@H]2C(=O)N[C@@H](C)c2ccc(-c3scnc3C)cc2)C(C)C)on1)OCC ; CCOC(COc1cc([C@H](C(=O)N2C[C@H](O)C[C@H]2C(=O)N[C@@H](C)c2ccc(-c3scnc3C)cc2)C(C)C)on1)OCC ; Cc1ncsc1-c1ccc([C@H](C)NC(=O)[C@@H]2C[C@@H](OP(=O)(O)O)CN2C(=O)[C@@H](c2cc(OCC=O)no2)C(C)C)cc1 ; C[C@@H]1CNCCN1CCCc1cc(N2C3CCC2CN(c2cc(-c4ccccc4OCc4ccc(NC(=O)[C@H](CCCCN(C)C)NC(=O)C5(C(=O)O)CCC5)cc4)nnc2N)C3)ccn1]

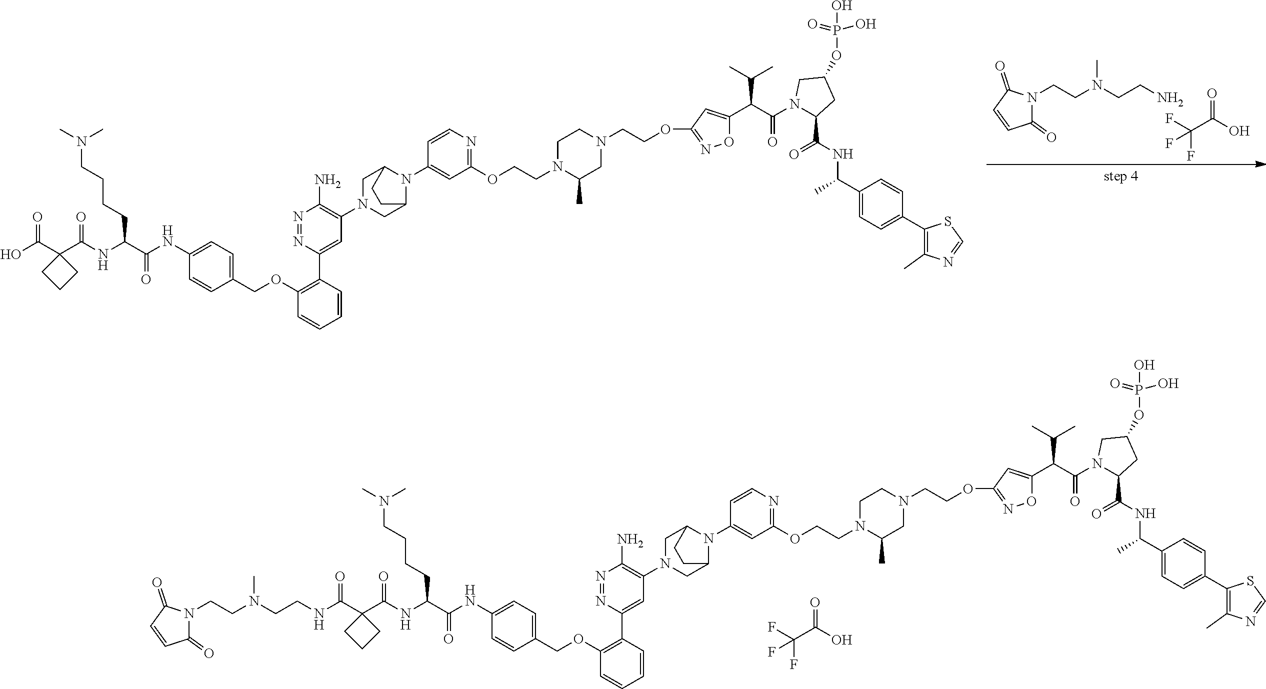

### Step 1: di-tert-butyl ((3R,5S)-1-((R)-2-(3-(2,2-diethoxyethoxy)isoxazol-5-yl)-3-methylbutanoyl)-5-(((d)-1-(4-(4-methylthiazol-5-yl)phenyl)ethyl)carbamoyl)pyrrolidin-3-yl) phosphate

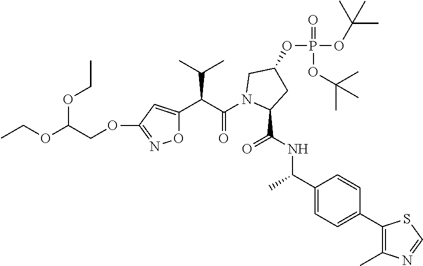 [SMILES: CCOC(COc1cc([C@H](C(=O)N2C[C@H](OP(=O)(OC(C)(C)C)OC(C)(C)C)C[C@H]2C(=O)N[C@@H](C)c2ccc(-c3scnc3C)cc2)C(C)C)on1)OCC]

Under nitrogen, to a solution of (2S,4R)-1-((R)-2-(3-(2,2-diethoxyethoxy) isoxazol-5-yl)-3-methylbutanoyl)-4-hydroxy-N—((S)-1-(4-(4-methylthiazol-5-yl)phenyl) ethyl) pyrrolidine-2-carboxamide (500 mg, 0.81 mmol) and 1H-tetrazole (5.4 mL, 0.45 M in ACN) in THF (5 mL) was added di-tert-butyl diisopropylphosphoramidite (451 mg, 1.63 mmol) dropwise at 0° C. The reaction was stirred at room temperature overnight. Then tBuOOH (0.4 mL, 5 M in decane) was added at −30° C. and stirred at −30° C. for 30 min. Then the mixture was stirred at room temperature for 4 hours. The reaction was quenched with aqueous NaHSO3 and extracted with EtOAc. The combined organic layer was washed with water and brine. The organic layer was dried over anhydrous sodium sulfate and concentrated under vacuum. The residue was purified by pre-packed C18 column (solvent gradient: 0-70% ACN in water (0.05% NH₄HCO₃)) to yield 340 mg (51%) of the title compound as a yellow oil. LC-MS: (ESI, m/z): [M+H]=807.

### Step 2: (3R,5S)-1-((R)-3-methyl-2-(3-(2-oxoethoxy)isoxazol-5-yl)butanoyl)-5-(((S)-1-(4-(4-methylthiazol-5-yl)phenyl)ethyl)carbamoyl)pyrrolidin-3-yl dihydrogen phosphate

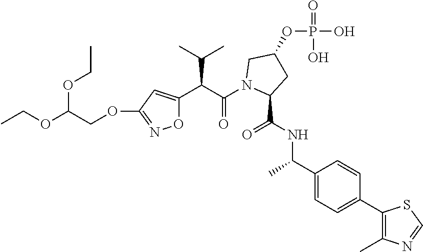 [SMILES: CCOC(COc1cc([C@H](C(=O)N2C[C@H](OP(=O)(O)O)C[C@H]2C(=O)N[C@@H](C)c2ccc(-c3scnc3C)cc2)C(C)C)on1)OCC]

Under nitrogen, a solution of di-tert-butyl ((3R,5S)-1-((R)-2-(3-(2,2-diethoxyethoxy)isoxazol-5-yl)-3-methylbutanoyl)-5-(((S)-1-(4-(4-methylthiazol-5-yl)phenyl)ethyl)carbamoyl)pyrrolidin-3-yl) phosphate (150 mg, 0.185 mmol) in HCOOH (0.5 mL) and water (0.5 mL) was stirred at 60° C. for 1 h. The solution was concentrated under vacuum to afford 121 mg (crude) of the title compound as a solid. LC-MS: (ESI, m/z): [M+H]⁺=695.

### Step 3: 1-(((2S)-1-((4-((2-(6-Amino-5-(8-(2-(2-((R)-2-methyl-4-(2-((5-((R)-3-methyl-1-((2S,4R)-2-(((S)-1-(4-(4-methylthiazol-5-yl)phenyl)ethyl)carbamoyl)-4-(phosphonooxy)pyrrolidin-1-yl)-1-oxobutan-2-yl)isoxazol-3-yl)oxy)ethyl)piperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid

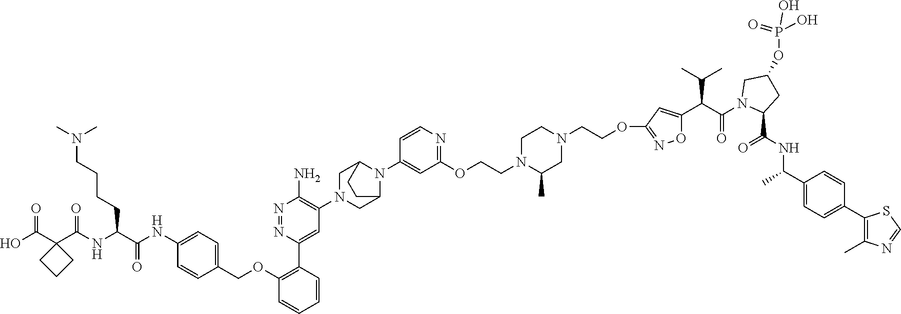 [SMILES: Cc1ncsc1-c1ccc([C@H](C)NC(=O)[C@@H]2C[C@@H](OP(=O)(O)O)CN2C(=O)[C@@H](c2cc(OCCN3CCN(CCOc4cc(N5C6CCC5CN(c5cc(-c7ccccc7OCc7ccc(NC(=O)[C@H](CCCCN(C)C)NC(=O)C8(C(=O)O)CCC8)cc7)nnc5N)C6)ccn4)[C@H](C)C3)no2)C(C)C)cc1]

A solution of 1-(((2S)-1-((4-((2-(6-amino-5-(8-(2-(2-((R)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (100 mg, 0.110 mmol), (3R,5S)-1-((R)-3-methyl-2-(3-(2-oxoethoxy)isoxazol-5-yl)butanoyl)-5-(((S)-1-(4-(4-methylthiazol-5-yl)phenyl)ethyl)carbamoyl)pyrrolidin-3-yl dihydrogen phosphate (121 mg, 0.190 mmol) and NaOAc (13.6 mg, 0.170 mmol) in methyl alcohol (1.2 mL) and dichloromethane (0.4 mL) was stirred at room temperature for 1 hours. Then NaBH₃CN (34.7 mg, 0.550 mmol) was added and stirred at room temperature for 0.5 hours. Water was added to quench the reaction. The solvent was concentrated under vacuum. The product was purified by Prep-HPLC (Column: YMC-Actus Triart C18 ExRS, 30*150 mm, 5 μm; Mobile Phase A: Water (10 mmol/L NH₄HCO₃), Mobile Phase B: ACN; Flow rate: 60 mL/min; Gradient: 36% B to 60% B in 9 min, 60% B; Wavelength: 254/220 nm; RT1(min): 7.63) to afford 30.0 mg (18% yield) of the title compound as a white solid. LCMS (ESI) [M+H]=1509.

### Step 4: (3R,5S)-1-((2R)-2-(3-(2-((3R)-4-(2-((4-(3-(3-amino-6-(2-((4-((S)-6-(dimethylamino)-2-(1-((2-((2-(2,5-dioxo-2,5-dihydro-1H-pyrrol-1-yl)ethyl)(methyl)amino)ethyl)carbamoyl)cyclobutane-1-carboxamido)hexanamido)benzyl)oxy)phenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoyl)-5-(((S)-1-(4-(4-methylthiazol-5-yl)phenyl)ethyl)carbamoyl)pyrrolidin-3-yl dihydrogen phosphate (2,2,2-trifluoroacetic acid salt)

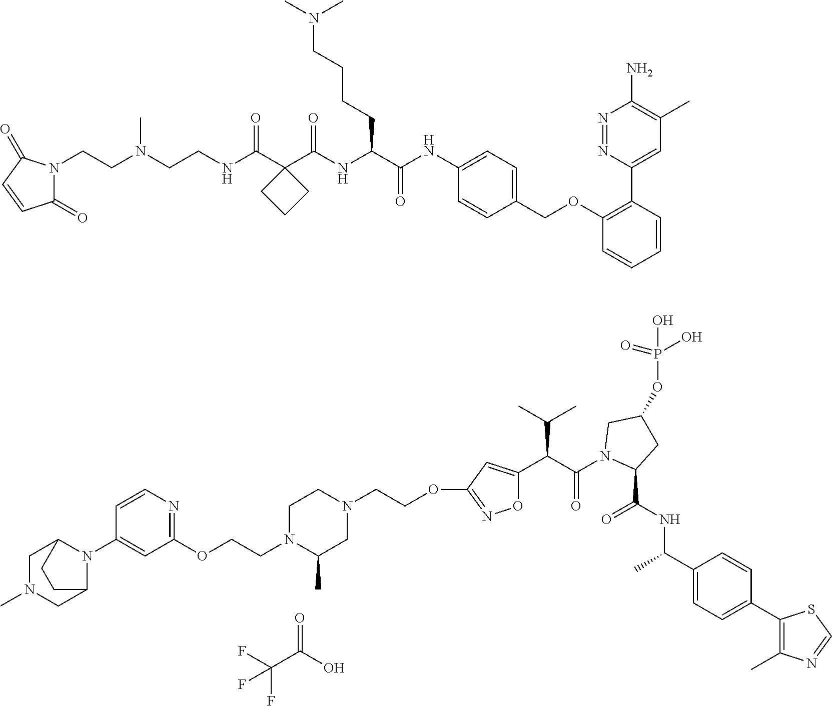

To a solution of 1-(((2S)-1-((4-((2-(6-amino-5-(8-(2-(2-((R)-2-methyl-4-(2-((5-((R)-3-methyl-1-((2S,4R)-2-(((S)-1-(4-(4-methylthiazol-5-yl)phenyl)ethyl)carbamoyl)-4-(phosphonooxy)pyrrolidin-1-yl)-1-oxobutan-2-yl)isoxazol-3-yl)oxy)ethyl)piperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (30.0 mg, 0.0200 mmol), 1-(2-((2-aminoethyl)(methyl)amino)ethyl)-1H-pyrrole-2,5-dione (2,2,2-trifluoroacetic acid salt) (12.3 mg, crude) and DIPEA (25.6 mg, 0.200 mmol) in DMF (1 mL) was added HATU (9.07 mg, 0.0200 mmol) at room temperature. The reaction was stirred at room temperature for 10 minutes. The crude was purified by Prep-HPLC (Column: Xselect CSH C18 OBD Column 30*150 mm 5 μm; Mobile Phase A: Water (0.05% TFA), Mobile Phase B: ACN; Flow rate: 60 mL/min; Gradient: 12% B to 24% B in 10 min, 24% B; Wavelength: 254/220 nm; RT1(min): 10) to afford 8.70 mg (24% yield) of L1Hy-PE-CIDE-6 as a white solid. LCMS (ESI) [M+H]=1688. ¹H NMR (300 MHz, DMSO-d6) δ 10.26 (s, 1H), 8.99 (s, 1H), 8.50 (d, J=7.5 Hz, 1H), 8.15 (t, 1H), 7.87 (d, J=6.5 Hz, 2H), 7.65 (d, J=8.3 Hz, 2H), 7.61-7.51 (m, 2H), 7.50-7.30 (m, 8H), 7.20-6.90 (m, 5H), 6.62 (s, 1H), 6.14 (s, 1H), 5.99 (s, 1H), 5.09 (s, 2H), 4.92 (t, J=7.2 Hz, 1H), 4.79 (s, 1H), 4.55-4.25 (m, 8H), 3.80-3.65 (m, 8H), 3.25-2.95 (m, 17H), 2.70 (s, 3H), 2.62 (s, 6H), 2.48-2.38 (m, 9H), 2.20-1.55 (m, 13H), 1.52-1.30 (m, 5H), 1.30-1.15 (m, 4H), 0.85 (d, J=6.4 Hz, 3H), 0.85-0.70 (m, 3H).

### Example 9

### Conjugate of L1-CIDE to an Antibody

The cysteine-engineered antibody (THIOMAB™), in 10 mM succinate, pH 5, 150 mM NaCl, 2 mM EDTA, was pH-adjusted to pH 7.5-8.5 with 1M Tris. 3-16 equivalents of a L1-CIDE (containing a thiol-reactive maleimide group) was dissolved in DMF or DMA (concentration=10 mM) and was added to a reduced, reoxidized, and pH-adjusted antibody. The reaction was incubated at room temperature or 37° C. and monitored until completion (1 to about 24 hours) as determined by LC-MS analysis of the reaction mixtures. When the reactions were complete, the Ab-CIDEs were purified by one or any combination of several methods, the goal being to remove remaining unreacted linker-drug intermediates and aggregated proteins (if present at significant levels).

In one example, the Ab-CIDEs were diluted with 10 mM histidine-acetate, pH 5.5 until the final pH was approximately 5.5 and were purified by S cation exchange chromatography using either HiTrap S columns connected to an Akta purification system (GE Healthcare) or S maxi spin columns (Pierce). Alternatively, the Ab-CIDEs were purified by gel filtration chromatography using an S200 column connected to an Akta purification system or Zeba spin columns. Dialysis was used to purify the conjugates.

The THIOMAB™ Ab-CIDEs were formulated into 20 mM His/acetate, pH 5, with 240 mM sucrose using either gel filtration or dialysis. The purified Ab-CIDEs were concentrated by centrifugal ultrafiltration and filtered through a 0.2-μm filter under sterile conditions and were frozen at −20° C. for storage.

### Example 10

### Cell Assays to Determine DC50 and Dmax

HCC515 and H1944 cells were plated in 384 well plates at 4000 and 2500 cells/well, respectively. The next day Ab-CIDEs were added. Following 24 h of drug treatment the cells were fixed with 4% formaldehyde for 15 minutes. The plates were washed three time with PBS. The cells were incubated with IF blocking solution (10% FCS, 1% BSA, 0.1% Triton, 0.01% Azide, X-100 in PBS). After 1.5 h a 2× solution of primary antibody diluted in IF blocking buffer: BRM (Cell signaling Cat #11966, 1:2000) was added. The plates were incubated over night at 4° C. The following morning cells were washed three time with PBS. Cells were then incubated with secondary antibodies (rabbit-Alexa 488 A21206 (1:2000)) for 1 h at room temperature in the dark. Hoechst H3570 at 1:5000 was added to the wells and the plates were incubated for an additional 30 minutes. Plates were wash 3×PBS and image on Opera Phenix™ High Content Screening System. Using nuclear staining as a mask, nuclear BRM mean signal intensity was quantified.

Data are shown in Table 1 below. The data evidence the successful antibody targeting strategies disclosed herein. Negative controls: Anti-TROP2 does not interact with NCI-H1944 cells, whereas anti-TfR2 does. The data show that several Ab-L1-CIDEs have both desirably low DC50 and desirably high DCmax values.

TABLE 1

| | | | DC50 | | BRM | |
|---|---|---|---|---|---|---|
| | | | mg/ml | DCmax | deg | BRM |
| | | | (NCl- | (NCl- | (%, | deg/ |
| L1-CIDE | Target | DAR | H1944) | H1944) | 96 h) | Dose |
| L1-PE-CIDE-3 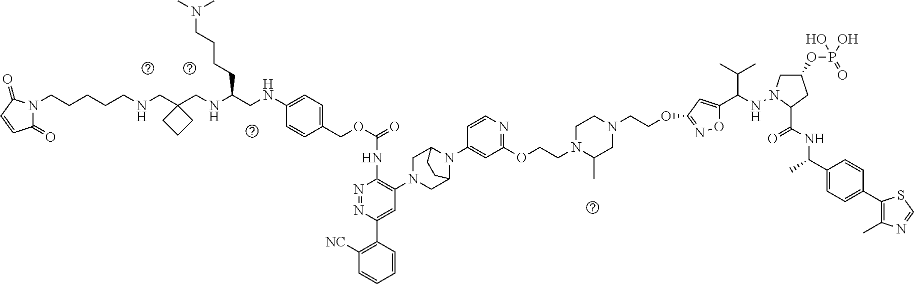 | TfR2 | 6.18 | 0.269 | 87 | 87 | 8.7 |
| Non-phosphorylated 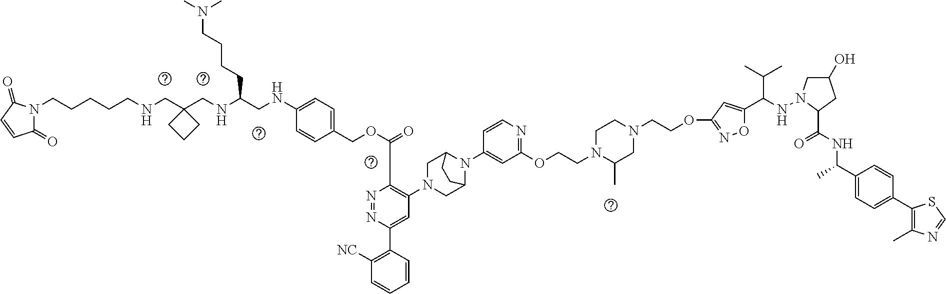 [SMILES: Cc1ncsc1-c1ccc([C@H](C)NC(=O)C2CC(O)CN2NC(c2cc(OCCN3CCN(CCOc4cc(N5C6CCC5CN(c5cc(-c7ccccc7C#N)nnc5CC(=O)OCc5ccc(NC[C@H](CCCCN(C)C)NCC7(CNCCCCCN8C(=O)C=CC8=O)CCC7)cc5)C6)ccn4)C(C)C3)no2)C(C)C)cc1] | TfR2 | 5.60 | ND | ND | 96 | 9.6 |
| L1-PP-CIDE-1 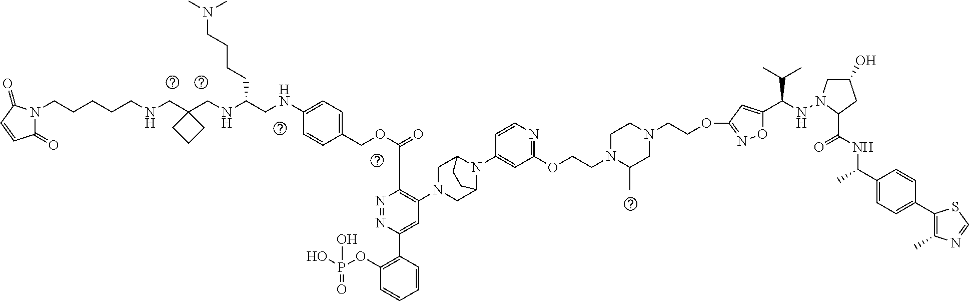 | TfR2 | 5.89 | 0.42 | 90 | ND | ND |
| L1-PE-CIDE-4 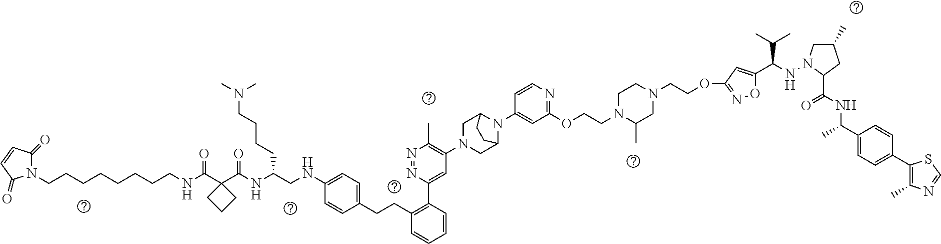 | TfR2 | 5.77 | 0.26 | 88 | 90 | 9 |
| Non-phosphorylated 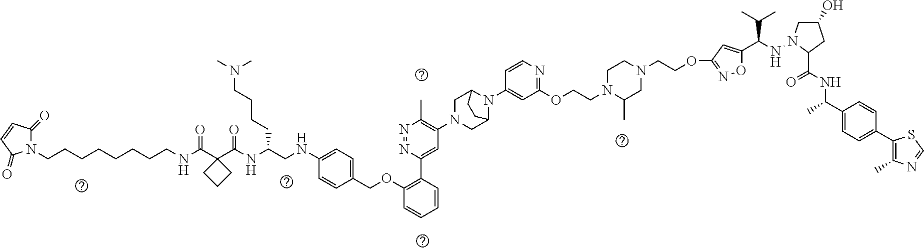 | TfR2 | 5.41 | 0.15 | 74 | 84 | 8.4 |
| Non-phosphorylated 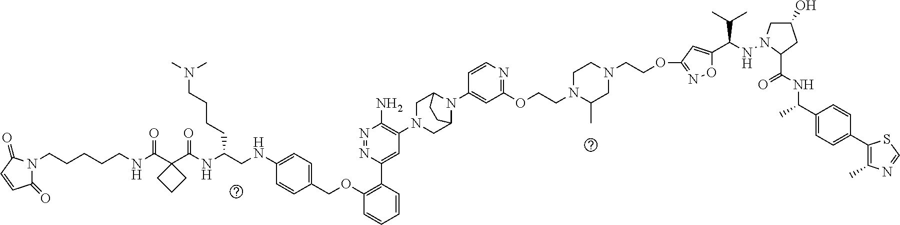 | TfR2 | 5.98 | 0.11 | 90 | ND | ND |
| L1-PE-CIDE-5 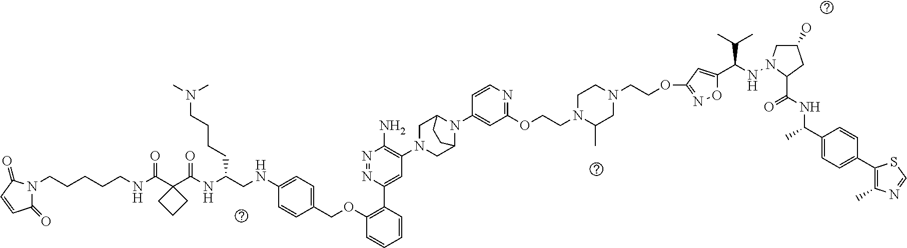 | TfR2 | 5.80 | 0.19 | 89 | ND | ND |
| L1-PP-CIDE-2 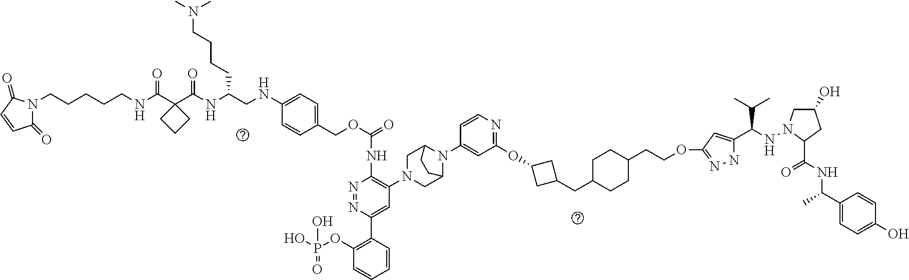 | TfR2 | 13.75 | 0.14 | 75 | ND | ND |
| Non-phosphorylated 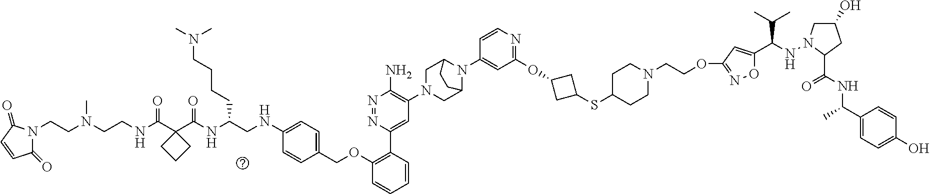 | TfR2 | 13.75 | 0.14 | 75 | ND | ND |
| L1-PE-CIDE-1 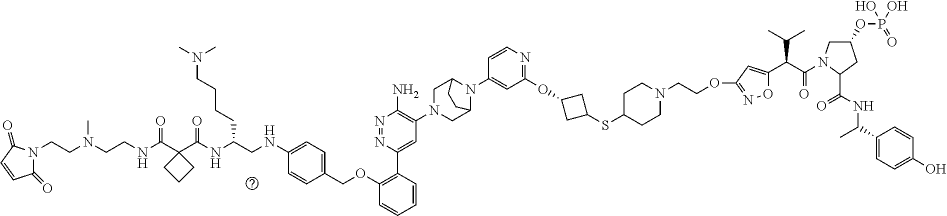 | TfR2 | 13.13 | ND | 42 | ND | ND |
| L1-PE-CIDE-2 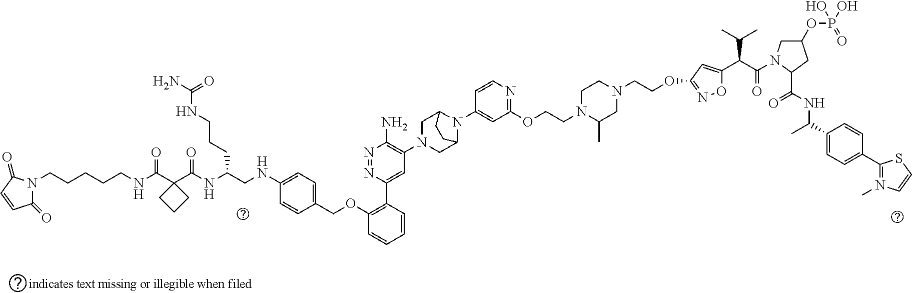 | Trop2 | 6.00 | ND | ND | ND | ND |

### Example 11

### PLAU and KRT80 Inhibition In Vivo Assays

PLAU Inhibition is a measure of the suppression of the mRNA transcript associated with the PLAU gene in response to BRM-degrader treatment. BRM activity controls the production of this transcript. KRT80 is a downstream marker of BRM degradation. Therefore, both the PLAU and KRT80 assays are measures of in vivo BRM degradation.

PK/PD HCC515 Tumor Assays: The PK/PD effects of anti-CD71-BRM Ab-CIDEs were evaluated in a mouse xenograft model of HCC515 human non-small cell lung carcinoma. The HCC515 cells were obtained from Genentech cell line repository. This cell line was authenticated by short tandem repeat (STR) profiling using the Promega PowerPlex 16 System and compared with external STR profiles of cell lines to confirm cell line ancestry.

To establish the model, tumor cells (5 million in 0.1 mL of Hank's Balanced Salt Solution) were inoculated subcutaneously to the flank of female C.B-17 SCID mice (Charles River Laboratories). When tumors reached the desired volume (200-300 mm3), mice were randomized into groups of n=5 each with similar distribution of tumor sizes, and received a single intravenous injection of vehicle (histidine buffer) or the test article through the tail vein. All anti-CD71-BRM Ab-CIDEs and the unconjugated antibody were formulated in the histidine buffer (20 mM histidine acetate pH 5.5, 240 mM sucrose, 0.02% Tween 20). The unconjugated BRM CIDE was formulated in 10% hydroxypropyl-beta-cyclodextrin, 50 mM sodium acetate, pH4.

At four days post-dose, mice were euthanized and tumors and whole blood were collected. Tumors were excised and split into two aliquots prior to being flash frozen in liquid nitrogen. One aliquot was used to measure level of released BRM CIDE and the other aliquot was used to evaluate the modulation of downstream PD markers. Whole blood was collected by terminal cardiac puncture under a surgical plane of anesthesia, and into tubes containing lithium heparin. Blood was allowed to sit on wet ice until centrifugation (within 15 min of collection). Samples were centrifuged at 10,000 rpm for 5 min at 4° C., and plasma was collected, placed on dry ice, and stored at −70° C. until analysis for linker stability and total antibody pharmacokinetics.

qRT-PCR assay for KRT80 and PLAU: For qRT-PCR 15-30 mg of frozen tissue was homogenized in 300 ul of lysis buffer with agitation in a Bullet Blender (Next Advance). RNA was prepared by MagMax mirVana Total RNA Isolation Kit (Applied Biosystems). Gene expression level was detected with FAM probes (ThermoFisher Scientific) using the Taqman One-Step RT-PCR Master Mix Reagents kit (ThermoFisher Scientific). Analysis was performed using 7900HT SDS (ThermoFisher Scientific). Expression levels are presented relative to the housekeeping gene, GusB (2-ΔCt).

| | |
|---|---|
| GusB | Hs00939627_m1 |
| Plau | Hs01547051_g1 |
| KRT80 | Hs01372363_g1 |
| IL32 | Hs00992441_m1 |

Data are shown in Table 2.

| | PLAU | KRT80 |
|---|---|---|
| | inhib. | inhib. |
| L1-CIDE | (%) | (%) |
| L1-PE-CIDE-3 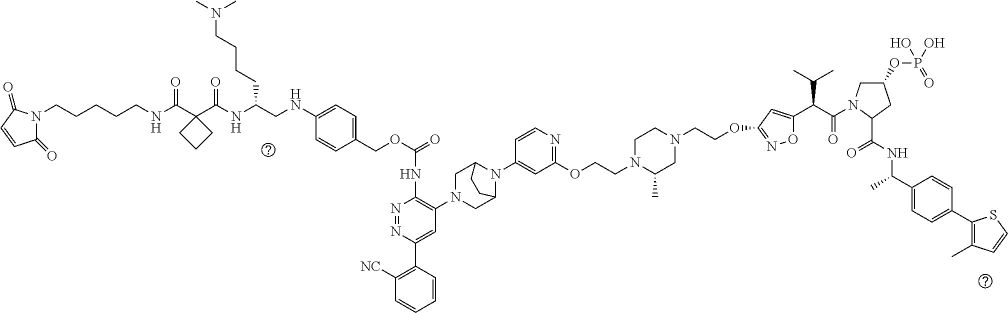 | 96 | 70 |
| Non-phosphorylated 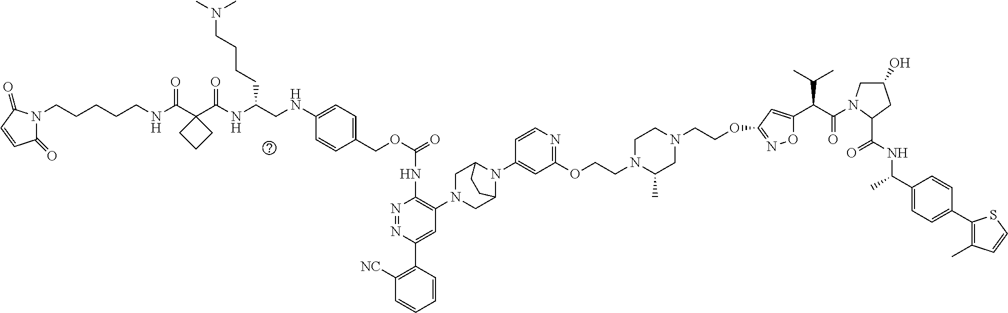 | 89 | 42 |
| L1-PE-CIDE-4 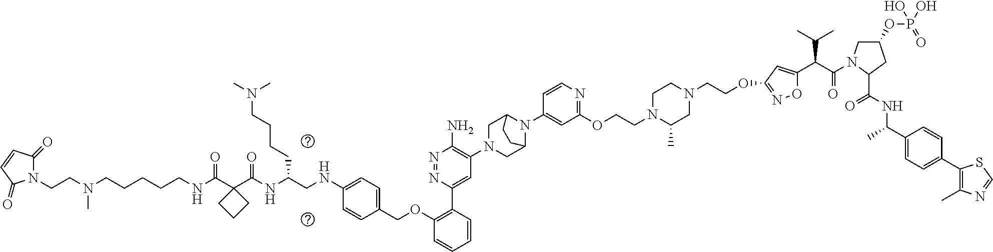 | 83 | 34 |
| Non-phosphorylated 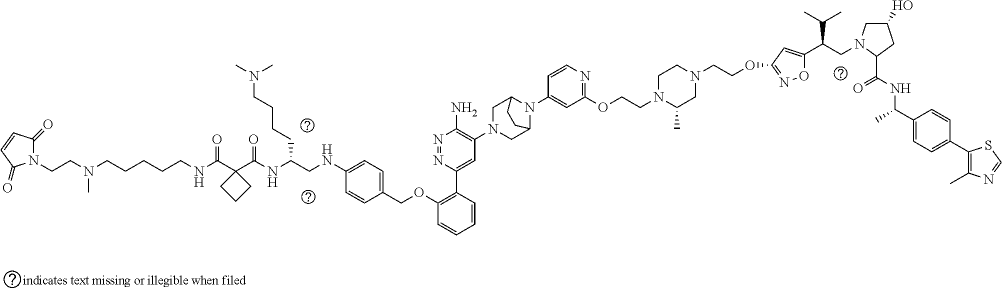 | 65 | 9 |

Efforts have been made to ensure accuracy with respect to numbers used (e.g. amounts, temperature, etc.) but some experimental errors and deviations should be accounted for.

One skilled in the art will recognize many methods and materials similar or equivalent to those described herein, which could be used in the practicing the subject matter described herein. The present disclosure is in no way limited to just the methods and materials described.

Unless defined otherwise, technical and scientific terms used herein have the same meaning as commonly understood by one of ordinary skill in the art to which this subject matter belongs.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

As used herein, the term “about,” when referring to a value is meant to encompass variations of, in some embodiments±50%, in some embodiments±20%, in some embodiments±10%, in some embodiments ±5%, in some embodiments±1%, in some embodiments ±0.5%, and in some embodiments±0.1% from the specified amount, as such variations are appropriate to perform the disclosed methods or employ the disclosed compositions.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller rangers is also encompassed, subject to any specifically excluded limit in the stated range. Where the stated range includes one or both of the limits, ranges excluding either or both of those included limits are also included.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation.

1. A conjugate having the chemical structure:
Ab-(L1-D)ⱼ,
wherein,
D is a CIDE having the structure:
E3LB-L2-PB,
wherein,
E3LB is an E3 ligase binding (E3LB) ligand covalently bound to L2,
wherein the E3 ligase is von Hippel-Lindau (VHL) tumor suppressor protein;
L2 is a linker covalently bound to E3LB and PB;
PB is a protein binding group covalently bound to L1 and to L2;
wherein, at least one phosphate moiety is covalently bound to D;
Ab is an antibody covalently bound to L1;
L1 is a linker covalently bound to Ab and D; and
j has a value of from about 1 to about 16.

2. The conjugate of claim 1, wherein one phosphate moiety is covalently bound to D at either the E3LB or the PB.

3. The conjugate of claim 1, wherein L1 is a peptidomimetic linker covalently bound to Ab and to the PB of D.

4. The conjugate of claim 1, wherein L1 is a peptidomimetic linker covalently bound to Ab and to the E3LB of D.

5. The conjugate of claim 1, wherein L1 is selected from the group consisting of:
i)
wherein 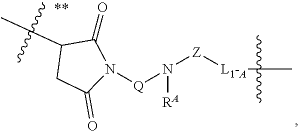
indicates the point of attachment to Ab; 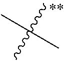
Z is —(CH₂)ₚ— or —CH₂—(CH₂—O—CH₂)ₚ—CH₂—, wherein p is 1, 2, 3, 4, 5 or 6;
R^{A} is hydrogen, C₁₋₆ alkyl, or —(CH₂)ᵥ-aryl, wherein, v is 0 or 1;
Q is selected from the group consisting of:
a) 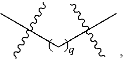
wherein, q is 1, 2, 3 or 4; and
b) 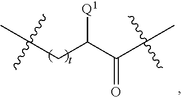
wherein, t is 0, 1, 2, 3 or 4; and
Q¹ is hydrogen, 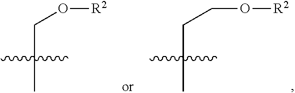
wherein R² is hydrogen, halo(C₁₋₆)alkyl or C₁₋₆ alkyl; and
L_{1-A} is: 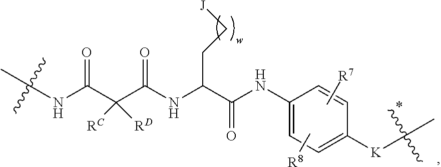
wherein, 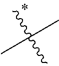
indicates the attachment point to D; and
ii) 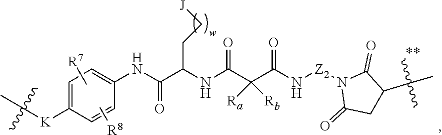
wherein 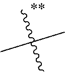
indicates the attachment point to Ab;
Z₂ is a C₁₋₁₂ alkylene or —[CH₂]_{g}—O—[CH₂]ₕ—, wherein g and h are each independently 0, 1 or 2;
w is 0, 1, 2, 3, 4 or 5;
J is selected from the group consisting of C₁₋₅ alkyl, —N(Rₓ)(R_{y}), —C(O)NH₂, —NH—C(O)—NH₂, and —NHC(═NH)NH₂, wherein, Rₓ and R_{y} are each independently selected from hydrogen and C₁₋₃alkyl;
K is selected from the group consisting of C₁₋₃alkylene, —CH(R)—, —C(O)—, —C(O)—O—CH(R)—, —CH₂—O—C(O)—, —CH₂—O—C(O)—NH—CH₂—, and —CH₂—O—C(O)—R—[CH₂]_{q}—O—, wherein R is hydrogen, C₁₋₃alkyl, N(Rₓ)(R_{y}), —O—N(Rₓ)(R_{y}) or C(O)—N(Rₓ)(R_{y}), wherein q is 0, 1, 2, or 3, and wherein Rₓ and R_{y} are each independently selected from hydrogen and C₁₋₃alkyl, or Rₓ and R_{y}, together with the nitrogen to which each is attached, form an optionally substituted 5- to 7-member heterocyclyl;
Rₐ, R_{b}, R^{C} and R^{D} are each independently selected from hydrogen and C₁₋₃alkyl, or Rₐ and R_{b}, together with the carbon to which each is attached, form an optionally substituted C₃₋₆cycloalkyl; and
R₇ and R₈ are each independently hydrogen, halo, C₁₋₅ alkyl, C₁₋₅ alkoxy or hydroxyl.

6. The conjugate of claim 5, wherein K of L1 is selected from the group consisting of:

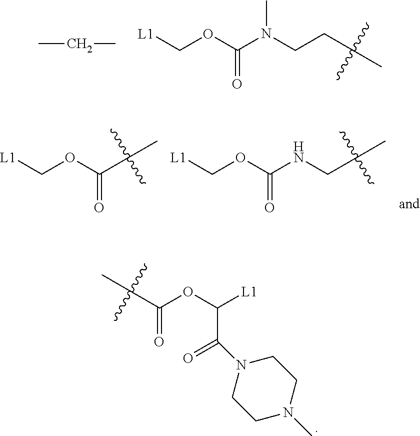

7. The conjugate of claim 1, wherein L1 is selected from the group consisting of:

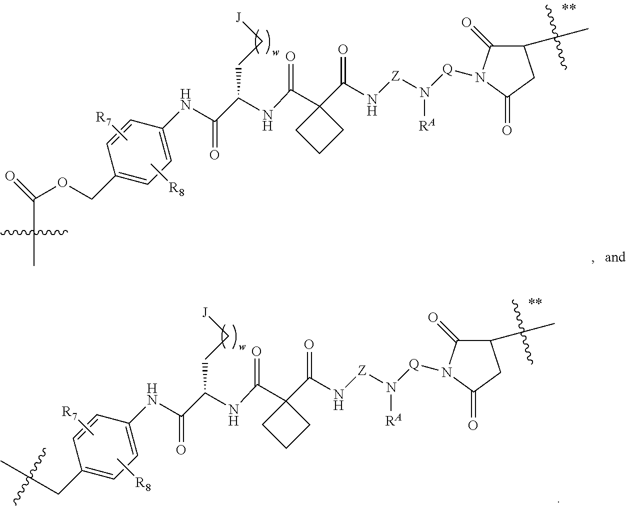

8-11. (canceled)

12. The conjugate of claim 1, wherein L1 is selected from the group consisting of: 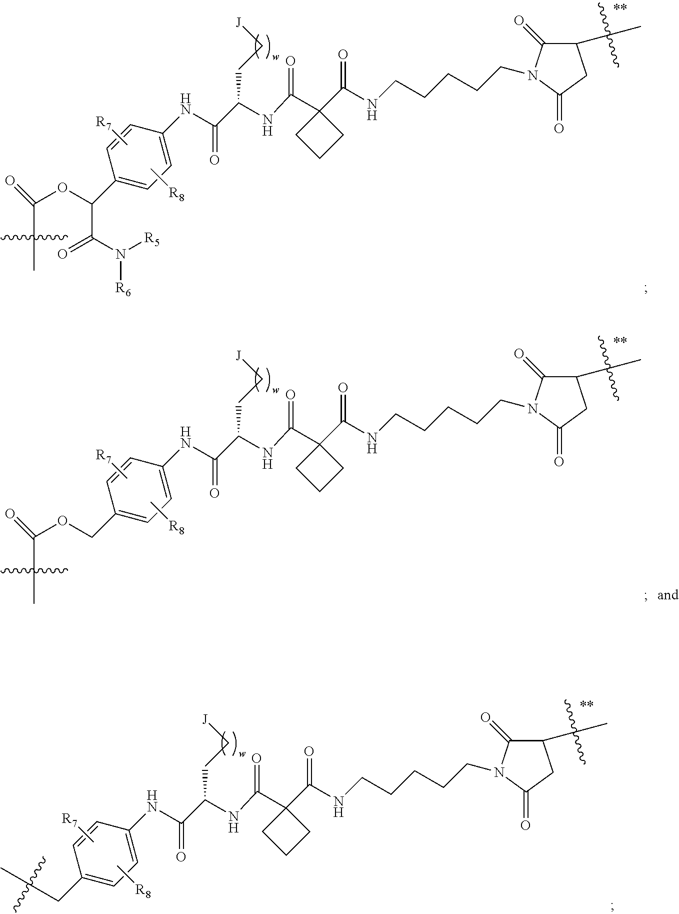
wherein, R₅ and R₆ are independently hydrogen or C₁₋₅ alkyl; or R₅ and R₆, together with the nitrogen to which each is attached, form an optionally substituted 5- to 7-member heterocyclyl.

13. The conjugate of claim 12, wherein L1 is selected from the group consisting of:

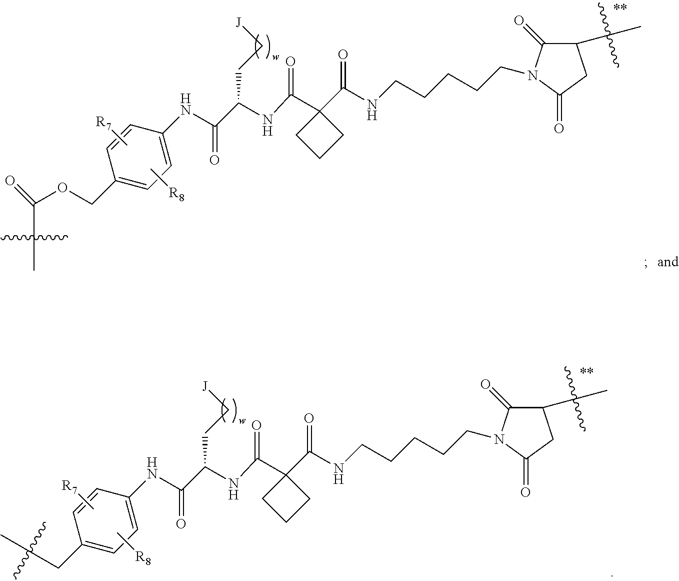

14. The conjugate of claim 12, wherein J is —NH—C(O)—NH₂ or —N(CH₃)₂.

15. (canceled)

16. The conjugate of claim 1, wherein L1 has the structure:

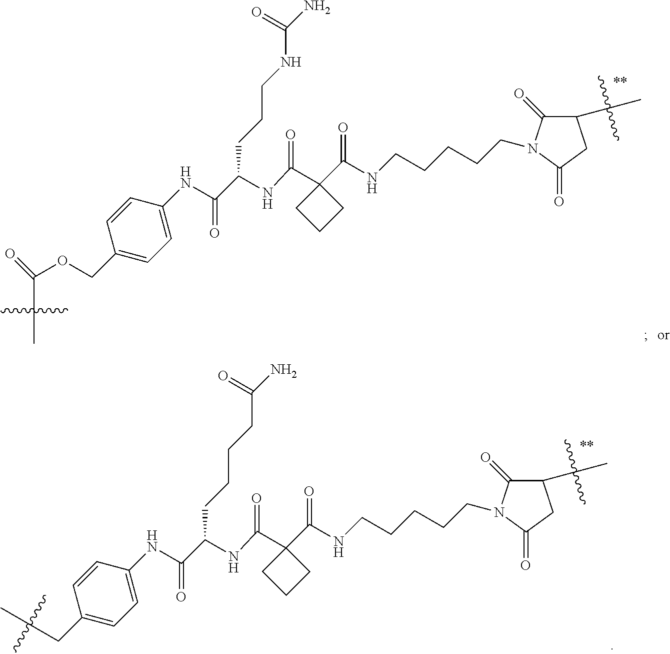 [SMILES: *C(=O)OCc1ccc(NC(=O)[C@H](CCCNC(N)=O)NC(=O)C2(C(=O)NCCCCCN3C(=O)CC(*)C3=O)CCC2)cc1 ; *Cc1ccc(NC(=O)[C@H](CCCCC(N)=O)NC(=O)C2(C(=O)NCCCCCN3C(=O)CC(*)C3=O)CCC2)cc1]

17. The conjugate of claim 1, wherein the phosphate moiety is selected from the group consisting of:
—(P═O)(OH)₂, —CH₂O(P═O)(OH)₂, —(P═O)(OH)O(P═O)(OH)₂ and —CH₂—O(P═O)(OH)O(P═O)(OH)₂.

18. The conjugate of claim 1, wherein the E3LB comprises a hydroxyproline residue, and wherein the phosphate moiety is covalently bound to E3LB through the oxygen of the hydroyproline residue.

19. (canceled)

20. The conjugate of claim 18, wherein the E3LB comprises:

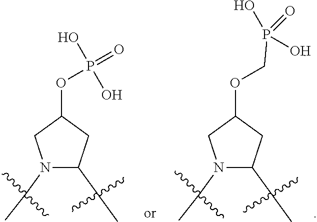 [SMILES: *C1CC(OP(=O)(O)O)CN1* ; *C1CC(OCP(=O)(O)O)CN1*]

21. (canceled)

22. The conjugate of claim 1, wherein the L1-D to which the antibody is conjugated is a residue of a compound selected from the group consisting of:

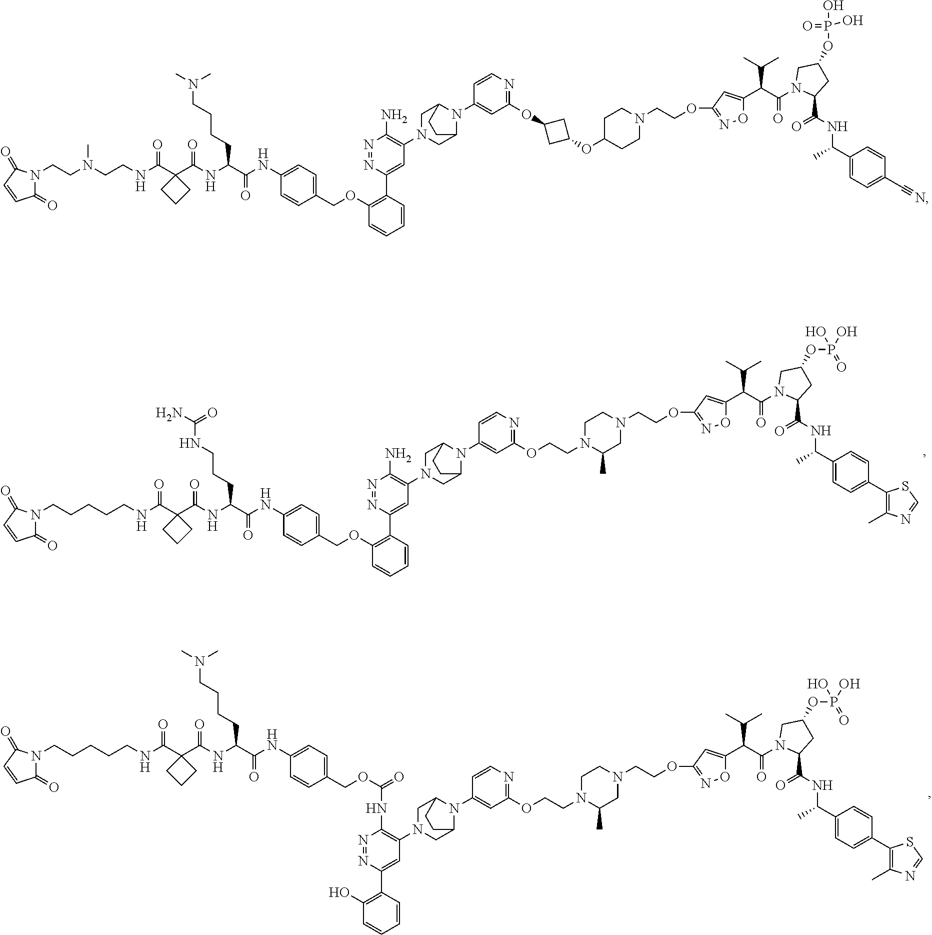

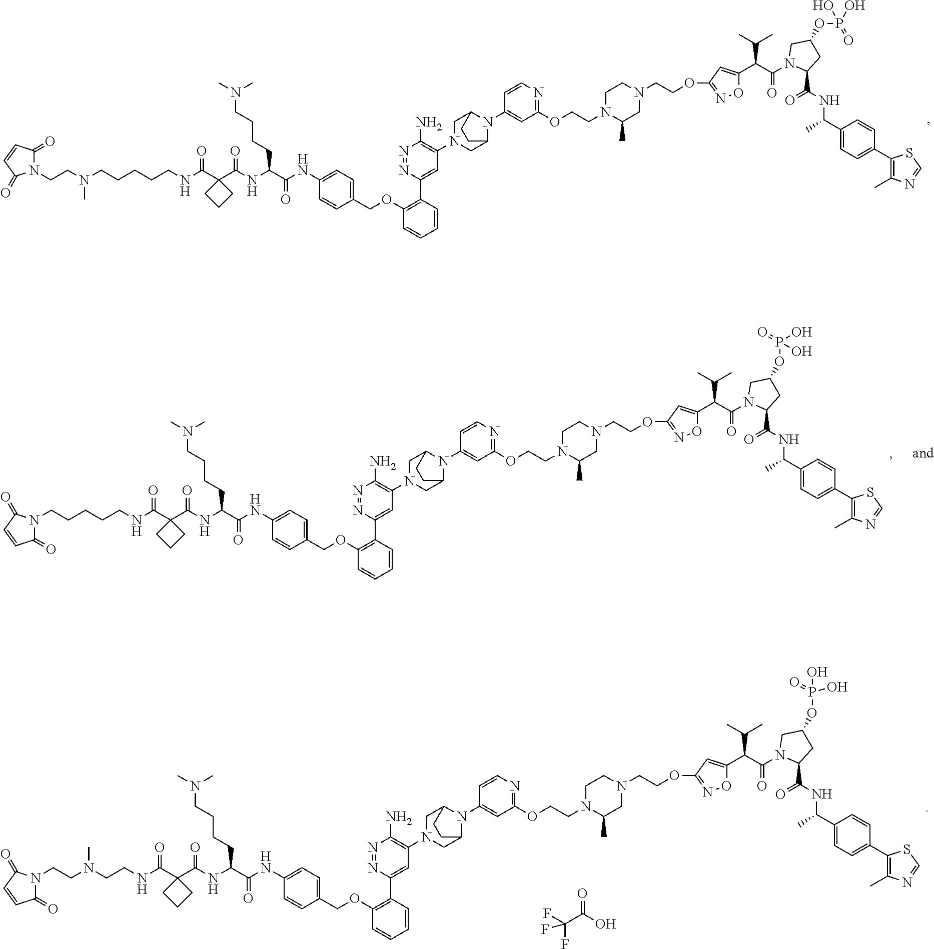

23. The conjugate of claim 1, wherein the PB comprises a hydroxyphenyl moiety, and wherein the phosphate moiety is covalently bound to PB through the oxygen of the hydroxyphenyl moiety.

24. (canceled)

25. (canceled)

26. The conjugate of claim 1, wherein the L1-D to which the antibody is conjugated is a residue of a compound selected from the group consisting of:

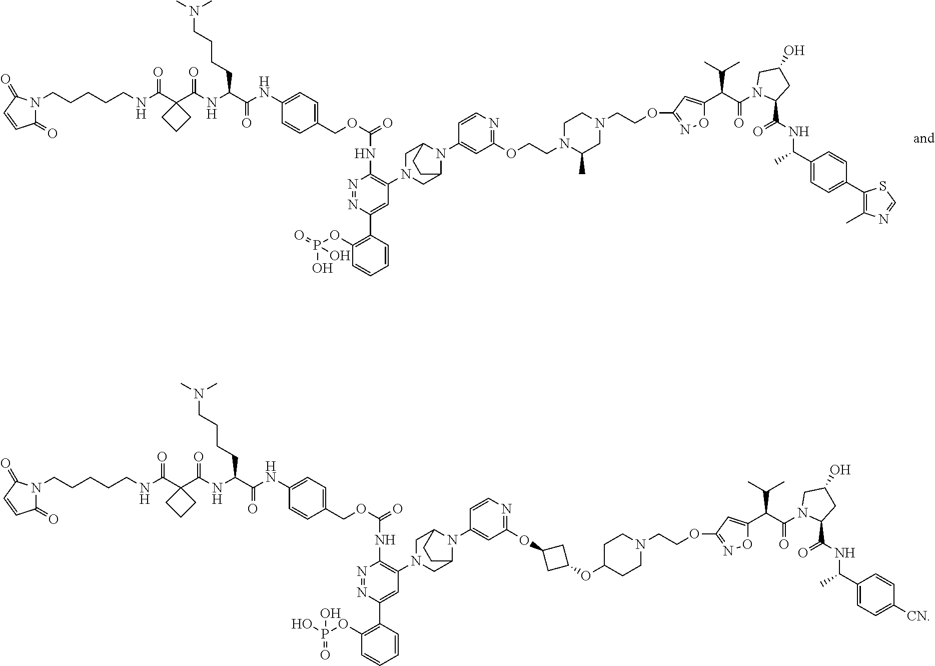 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7OP(=O)(O)O)nnc5NC(=O)OCc5ccc(NC(=O)[C@H](CCCCN(C)C)NC(=O)C7(C(=O)NCCCCCN8C(=O)C=CC8=O)CCC7)cc5)C6)ccn4)[C@H](C)C3)no2)C(C)C)cc1 ; CC(C)[C@@H](C(=O)N1C[C@H](O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OP(=O)(O)O)nnc5NC(=O)OCc5ccc(NC(=O)[C@H](CCCCN(C)C)NC(=O)C7(C(=O)NCCCCCN8C(=O)C=CC8=O)CCC7)cc5)C6)ccn4)C3)CC2)no1]

27. A pharmaceutical composition comprising the conjugate of claim 1 and one or more pharmaceutically acceptable excipients.

28. A method of treating a disease in a human in need thereof, comprising administering to said human an effective amount of the conjugate of claim 1.

29-31. (canceled)

32. A method of reducing the level of a target protein in a subject comprising administering the conjugate of claim 1 to said subject, wherein said PB portion binds said target protein, wherein ubiquitin ligase effects degradation of said bound target protein, and wherein the level of said target protein is reduced.
